(19) United States
(12) Patent Application Publication
(10) Pub. No.: US 2025/0034501 A1
(43) Pub. Date: Jan. 30, 2025

(54) AUTOMATED HOLLOW FIBER SYSTEM

(71) Applicant: Takeda Pharmaceutical Company Limited, Osaka (JP)

(72) Inventors: Nathan Moore, Lexington, MA (US); Fabio Fachin, Lexington, MA (US); Ryan Maloney, Lexington, MA (US)

(21) Appl. No.: 18/714,658

(22) PCT Filed: Dec. 8, 2022
(86) PCT No.: PCT/IB2022/061937

§ 371 Date: May 30, 2024

Related U.S. Application Data
(60) Provisional application No. 63/365,829, filed on Jun. 3, 2022.
(60) Provisional application No. 63/287,651, filed on Dec. 9, 2021.

(51) Int. Cl. C12M 1/12; C12M 1/00; C12M 1/36; C12M 3/06

(52) U.S. Cl. CPC C12M 25/10; C12M 23/16; C12M 29/04; C12M 29/16; C12M 41/48

(57) ABSTRACT

A computer-implemented method, that when executed on data processing hardware, causes the data processing hardware to perform operations at a hollow fiber transduction device. The operations include instructing a pump of the hollow fiber transduction device to execute one of the steps of the recipe to provide a first pressure at a first sensor where the first pressure is specified in the recipe. The operations also include receiving, from a first sensor of the hollow fiber transduction device, a fluid flow signal that indicates an operating parameter measured at the first sensor, comparing a first end trigger threshold of the one of the steps of the recipe against the received fluid flow signal, and instructing the pump to complete the one of the steps of the recipe based on the first end trigger threshold comparison.

### CROSS REFERENCE TO RELATED APPLICATIONS

This application claims priority to U.S. Provisional Application 63/287,651, filed on Dec. 9, 2021 and U.S. Provisional Application 63/365,829, filed on Jun. 3, 2022. The disclosure of these prior applications are considered part of the disclosure of this application and are hereby incorporated by reference in their entirety.

### TECHNICAL FIELD

This disclosure relates to an automated hollow fiber system for transduction of cells.

### BACKGROUND

Cell therapies take advantage of the natural transduction process, typically using virus particles modified for safety and functionality as a delivery vehicle (vector) for introducing genetic material into a patient's cells or cells from a donor, e.g., for manufacture of autologous or allogenic cell therapies. Viral vector transduction is currently the most frequently used method in cell therapy manufacturing for introducing therapeutic genetic material into a cell (such as an immune cell or a stem cell). Current transduction processes employed in the manufacture of cell therapies tend to be labor intensive and inefficient in the use of viral vectors, particularly at large scale, thereby resulting in high manufacturing costs for cell therapies and also extending the time required to produce such therapies. Accordingly, there is a need in the industry for more efficient and cost effective virus transduction processes for manufacturing cell therapies, in particular ex vivo cell therapies.

### SUMMARY

The present disclosure provides automated hollow fiber systems and methods for transduction of cells for the manufacture of cell therapies. The methods and systems described herein are less labor intensive, more cost effective and efficient and suitable for large scale manufacturing of cell therapies. Furthermore, the automated hollow fiber systems and methods described hereunder minimize risk of contamination during cell therapy manufacturing by automating the transduction process and result in a more consistent and uniform transduction of cells.

One aspect of the disclosure provides a computer-implemented method that, when executed on data processing hardware, causes the data processing hardware to perform operations at a hollow fiber transduction device. The hollow fiber transduction device has a pump and a plurality of sensors for executing a recipe that includes a plurality of steps. Here, each step has a first end trigger threshold that indicates whether the respective step of the recipe is complete. The operations include instructing the pump to execute one of the steps of the recipe to provide a first pressure at a first sensor of the plurality of sensors where the first pressure is specified in the recipe. The operations also include receiving, from the first sensor of the plurality of sensors, a fluid flow signal that indicates an operating parameter measured at the first sensor. The operations also include comparing a first end trigger threshold of the one of the steps of the recipe against the received fluid flow signal. The comparison of the first end trigger threshold indicates whether an operational condition is met. The operations also include instructing the pump to complete the one of the steps of the recipe based on the first end trigger threshold comparison.

Implementations of the disclosure may include one or more of the following optional features. In some implementations, each step of the recipe includes at least one of a valve configuration, a pump direction, a pump speed, or a step type. In some examples, instructing the pump to execute the one of the steps of the recipe to provide the first pressure at the first sensor of the plurality of sensors includes sending a pump speed signal to the pump to operate at a first pump speed. Here, instructing the pump to execute the one of the steps of the recipe to provide the first pressure at the first sensor of the plurality of sensors further includes sending a pump direction signal to the pump to operate in a first direction. In these examples, after instructing the pump to complete the one of the steps of the recipe based on the first end trigger threshold comparison, the operations may further include instructing the pump to execute another one of the steps of the recipe to provide a second pressure at the first sensor of the plurality of sensors where the second pressure specified in the recipe. Here, the other one of the steps of the recipe further instructs the pump to operate in a second direction opposite of the first direction.

In some implementations, the operations further include: waiting a duration of time corresponding to a timer value if the received fluid flow signal fails to satisfy the first end trigger threshold of the one of the steps of the recipe; receiving, from the first sensor of the plurality of sensors, a second fluid flow signal indicating the operating parameter measured at the first sensor; comparing the first end trigger threshold of the one of the steps of the recipe against the received second fluid flow signal; and instructing the pump to execute the one of the steps of the recipe again depending on the comparison. In these implementations, the operating parameter includes at least one of a pressure value or a fluid flow value.

The operations may further include determining whether the one of the steps of the recipe is a last step of the recipe. In some examples, the operations further include, when the one of the steps of the recipe is the last step of the recipe, completing execution of the recipe. In other examples, the operations further include, when the one of the steps of the recipe is the not the last step of the recipe, executing a next step of the recipe. The one of the steps of the recipe may include a second end trigger threshold that indicates whether a failure has occurred at the hollow fiber transduction device during execution of the respective step of the recipe. Here, each of the first and second end trigger thresholds include at least one of a pressure value, a timer value, an air quantity value, a fluid flow value, or a fluid-to-air ratio. In some examples, the operations further include comparing a second end trigger threshold of the one of the steps of the recipe against the received fluid flow signal and instructing the pump to bypass the one of the steps of the recipe based on the second end trigger threshold comparison.

Optionally, the operations may further include storing the recipe at memory hardware in communication with the data processing hardware. In some examples, the operations further include receiving a request to execute the recipe stored at the memory hardware, retrieving the recipe stored at the memory hardware, and executing, by the data processing hardware, the recipe. In some implementations, the operations further include receiving a request to modify at least one step of the recipe stored at the memory hardware and updating the at least one step of the recipe stored at the memory hardware.

Another aspect of the disclosure provides a system that includes a filter module defining an intra-capillary space and an extra-capillary space separated from the intra-capillary space by a porous membrane. Here, the filter module includes a pair of intra-capillary ports fluidly coupled to the intra-capillary space and a pair of extra-capillary ports coupled the extra-capillary space. The system also includes a first pump that includes a first pump inlet in communication with each of a cell source that has cells and a vector source that has a vector, and a first pump outlet in selective communication with each of the pair of intra-capillary ports and the pair of extra-capillary ports. The system also includes a second pump that includes a second pump inlet in communication with each of the cell source and the vector source, and a second pump outlet in selective communication with each of the pair of intra-capillary ports and the pair of extra-capillary ports. The system also includes a graphical user interface (GUI) in operative communication with each of the first pump and the second pump and is configured to receive a request from a user for operating the first pump and second pump according to a recipe that includes one or more steps.

Implementations of the disclosure may include one or more of the following optional features. In some implementations, the system further includes at least one valve disposed between the first pump outlet and a first intra-capillary port of the pair of intra-capillary ports, at least one valve disposed between the first pump outlet and a second intra-capillary port of the pair of intra-capillary ports, and a fluid sensor disposed between the first pump outlet and the first intra-capillary port of the pair of intra-capillary ports. Here, the valves operate according to the recipe. In some examples, the system further includes a valve disposed between the first pump inlet and the cell source and a bubble sensor disposed between the first pump inlet and the cell source. Here, the valve operates according to the recipe.

The system may further include at least one valve disposed between the second pump outlet and a first intra-capillary port of the pair of extra-capillary ports, at least one valve disposed between the second pump outlet and a second intra-capillary port of the pair of intra-capillary ports, and a fluid sensor disposed between the second pump outlet and the first intra-capillary port of the pair of extra-capillary ports. Here, the valves operate according to the recipe. Optionally, the GUI is further configured to display to the user the recipe from memory hardware in response to receiving the request.

Another aspect of the disclosure provides a system that incudes a filter module defining an intra-capillary space and an extra-capillary space separated from the intra-capillary space by a porous membrane. The filter module includes a pair of intra-capillary ports fluidly coupled to opposite ends of the intra-capillary space and a pair of extra-capillary ports coupled to opposite ends of the extra-capillary space. The system also includes a first pump that includes a first pump inlet in communication with each of a cell source that has cells and a vector source that has a vector, and a first pump outlet in selective communication with a first intra-capillary port of the pair of intra-capillary ports and a second intra-capillary port of the pair of intra-capillary ports. The system also includes a first fluid sensor disposed between the first pump outlet and the first intra-capillary port of the pair of the intra-capillary ports. The system also includes data processing hardware and memory hardware storing instructions that when executed on the data processing hardware cause the data processing hardware to perform operations according to a recipe for transducing the cells with the vector.

Implementations of the disclosure may include one or more of the following optional features. In some implementations, the operations include: operating the first pump at a first pump rate to provide a first flow of at least one of the cells or the vector to each of the pair of the intra-capillary ports of the filter module; obtaining, from the first fluid sensor, a first fluid flow signal indicating the first flow of at least one of the cells or the vector; comparing the first fluid flow signal against a first fluid flow parameter threshold; and operating the first pump at a second pump rate depending on the comparison. In these implementations, the operations may further include initiating a pump rate adjustment procedure depending on the comparison. The pump rate adjustment procedure includes stopping operation of the first pump for a predetermined period. The predetermined period may be a period of time. The predetermined period may also be a pressure adjustment period. In some examples, the pump rate adjustment procedure further includes operating the first pump in a reverse-flow state.

In some implementations, the operations further include operating the first pump at a third pump rate to provide a second flow of the other of the one of the cells or the vector to each of the pair of the intra-capillary ports of the filter module. In these implementations, the operations may further include instructing the first pump to alternate operation between operating at the first pump rate to provide the first flow of the at least one of the cells or the vector to each of the pair of the intra-capillary ports of the filter module and operating at the third pump rate to provide the second flow of the other of the one of the cells or the vector to each of the pair of the intra-capillary ports of the filter module. The operations may further include operating the first pump at the third pump rate to provide the second flow of the other of the one of the cells or the vector to each of the pair of the intra-capillary ports of the filter module after operating the first pump at the first pump rate to provide the first flow of the at least one of the cells or the vector to each of the pair of the intra-capillary ports of the filter module. In some examples, the operations further include: obtaining, from the first fluid sensor, a second fluid flow signal indicating the second flow of the other of the one of the cells or the vector; comparing the second fluid flow signal against a second fluid flow parameter threshold; and initiating a pump rate adjustment procedure dependent upon the comparison.

In some examples, the system further includes a transduction media source that includes transduction media in communication with the first pump inlet. In these examples, the operations further include operating the first pump at a third pump rate to provide a third flow of the transduction media to each of the pair of the intra-capillary ports. The system may further include a second pump that includes a second pump inlet in communication with each of the cell source and the vector source, and a second pump outlet in selective communication with each of the pair of extra-capillary ports. Here, the system may further include a culture media source that includes a culture media in communication with the first pump inlet and the second pump inlet. In some examples, the operations further include operating the first pump at a fourth pump rate to provide a fourth flow of the culture media to one of the intra-capillary ports and operating the second pump at a fifth pump rate to provide a fifth flow of the culture media to each of the pair of the extra-capillary ports.

In some implementations, the vector used for transduction of cells includes a non-viral vector or a viral vector. In some examples, the system further includes a mixer configured to receive at least one of a population of cells or the vector particles. In these examples, the operations further include mixing, using the mixer, the cells with the vector prior to loading the mixture of both into the filter module. The operations may further include generating a cell therapy product that includes one or more transduced cells by loading a population of cells and vector particles into the intra-capillary space thereby resulting in transduction of one or more of the cells in the population of cells in the in the intra-capillary space and harvesting the population of cells that include the one or more transduced cells from the intra-capillary space.

One aspect of the disclosure provides a computer-implemented method for manufacturing a cell therapy product that, when executed on data processing hardware, causes the data processing hardware to perform operations for executing a recipe comprising a plurality of steps at a hollow fiber transduction device. The hollow fiber transduction has a pump that includes a pump inlet in communication with each of a cell source that has cells and a vector source that has a vector and a first pump outlet in selective communication with a first intra-capillary port and a second intra-capillary port of a filter module. The operations include instructing the pump to execute one of the steps of the recipe to provide the cells and the vector at the first intra-capillary port and the second intra-capillary port of the filter module for transducing the cells. The operations also include instructing the pump to execute a subsequent step of the recipe to provide the transduced cells from the filter module to a harvest container in communication with the second intra-capillary port of the filter module.

Implementations of the disclosure may include one or more of the following optional features. In some implementations, the operations further include expanding the transduced cells in a suitable culture medium according to a step of the recipe. In some examples, the operations further include cryopreserving the transduced cells after expansion of the transduced cells according to a step of the recipe. In some implementations, at least fifty percent, sixty percent, seventy percent, eighty percent, or ninety percent or greater of the cells provided at the filter module are transduced. Optionally, the vector may include a viral-vector or a non-viral vector. The vector may include a chimeric antigen receptor (CAR) construct expressed on a surface of the transduced cells. In some examples, the operations further include separating the transduced cells from untransduced cells according to a step of the recipe.

The details of one or more implementations of the disclosure are set forth in the accompanying drawings and the description below. Other aspects, features, and advantages will be apparent from the description and drawings, and from the claims.

### DESCRIPTION OF DRAWINGS

FIGS. 1A-1C illustrate perspective views of a hollow fiber system.

FIG. 1D is a schematic view of a controller and a graphical user interface in communication with components of the hollow fiber system.

FIG. 2 is a schematic view of an example computing device that may be used to implement the systems and methods described herein.

FIG. 3A illustrates a schematic view of a hollow fiber system showing fluid flow paths.

FIG. 3B illustrates a horizontal cross-section of a simplified example of a hollow fiber of a filter module.

FIG. 3C illustrates a vertical cross-section of the simplified example of the hollow fiber of the filter module.

FIG. 3D illustrates a horizontal cross-section of a filter module that includes a plurality of hollow fibers.

FIG. 4 is a flowchart of an example arrangement of operations for a method of executing operating a graphical user interface in a user mode and a developer mode.

FIG. 5A is a schematic view of an example recipe.

FIG. 5B is a schematic view of an example end trigger threshold for the recipe of FIG. 5A.

FIG. 6 is a flowchart of an example arrangement of operations for a method of executing an initialization routine at the hollow fiber system.

FIG. 7 is a flowchart of an example arrangement of operations for a method of executing each step of a recipe.

FIG. 8 is a flowchart of an example arrangement of operations for a method of executing a pump rate adjustment procedure.

FIG. 9 is a flowchart of an example arrangement of operations for a method of operating the hollow fiber system according to a recipe.

FIG. 10 is a flowchart of an example arrangement of operations for a method of initiating a pump rate adjustment procedure while operating pumps at a first pump speed according to a recipe.

FIG. 11 is a flowchart of an example arrangement of operations for a method of initiating a pump rate adjustment procedure while operating pumps at a first fluid flow parameter according to a recipe.

FIGS. 12-24 illustrate schematic views of example fluid flow paths during execution of a recipe.

FIG. 25 illustrates a schematic view of a hollow fiber system showing fluid flow paths.

FIGS. 26-45 illustrate schematic views of example fluid flow paths through the hollow fiber system of FIG. 25 during execution of a recipe.

FIG. 46 illustrates a chart of cell and vector loading, where the chart illustrates the comparison of constant pressure and constant flow.

FIG. 47 illustrates a chart of hollow-fiber transduction efficiency by length.

FIG. 48 illustrates a chart of cell and vector loading, where the chart illustrates a comparison of premixed and sequential loads.

FIG. 49 illustrates a chart of hollow-fiber transduction of natural killer cells.

FIG. 50 illustrates a chart of optimization studies in the fully-automated platform demonstrating retrovirus transduction of γδT cells in the hollow-fiber system.

Like reference symbols in the various drawings indicate like elements.

### DETAILED DESCRIPTION

Transduction is a process through which viruses infect the cells of a target or host cell. Viruses naturally undergo the transduction process and have evolved to be very efficient at introducing genetic material into target cells. In order for transduction to occur, virus particles must come in physical contact with their target cells to first bind, enter, and finally introduce genetic material into the target cells. Binding occurs through specific protein-protein interactions, with the correct proteins needed on both the virus and target cell.

Cell therapies take advantage of the natural transduction process, using virus particles modified for safety and functionality as a delivery vehicle (vector) for introducing genetic materials into cells derived from a patient (e.g., in case of autologous cell therapies) or derived from a donor (e.g., in case of allogenic cell therapies). Viral vector transduction is currently the most frequently used method in cell therapy manufacturing for introducing genetic material into a cell.

Current industry approaches to viral transduction include static transduction systems, the use of chemical enhancers, and spinoculation. Each of these current industry approaches is further described below.

Viral transduction under static conditions is the most prevalent manner in which viral transductions are currently performed in the industry. Under standard static transduction methods, most transductions are performed in standard culture flasks or bags under static culture conditions. In this manner, viral vector is suspended in media containing a population of cells and transduction efficiency is largely left to chance. Transduction using standard static methods face various problems that result in inefficient transduction of the cells. For example, using static methods results in the presence of small vector particles that remain in suspension and are unable to reach target cells. This occurs, at least in part, because large cells quickly sediment to the floor of culture vessels. The end result using the static culture methods for transduction is that only a small fraction of vector particles are capable of reaching cells through diffusion alone. As a result, transduction efficiency is low and the quantity of viral vector needs to be high to achieve appreciable cellular transduction. This is because viral vector binding to a target cell is determined by receptor/ligand expression and physical contact. The transduction rate is thus proportional to the local concentration of virus for a given cell. Requiring large quantities of viral vector to achieve a satisfactory transduction rate can be costly and also create inefficiencies in the overall cell therapy manufacturing process.

Another standard method for cellular transduction is the use of spinoculation. Spinoculation refers to centrifugal inoculation of cells. Spinoculation reduces the volume occupied by cells. This technique has been shown to have various negative aspects including, for example, damage to cells, difficulty in scaling up, and it is generally less effective for small vectors.

Yet another method for enhancing the transduction efficiency of viruses, particularly retroviruses, is by use of cell adhesive substances or chemical enhancers that bind to retroviruses, such as fibronectin or a fibronectin fragment CH-296 [RETRONECTIN® (recombinant human fibronectin fragment) or retronectin]. This method requires adding a solution containing a retroviral vector to a vessel coated with retronectin followed by incubation for a certain period of time to allow only the viral vector to bind onto retronectin, removing supernatant, which contains inhibitory substances against virus infection, and then adding target cells. Coating of vessel surface with retronectin is tedious and makes this method rather costly. In addition, this method is difficult to scale up when gene transfer into a large number of cells is required.

Implementations herein are directed towards a fully automated hollow fiber system for performing transduction of cells. In some implementations, the transduction methods described herein obviate the need to use any chemical enhancers or cell adhesives. In other implementations, transduction methods described herein may employ the use of chemical enhancers or cell adhesives for efficient transduction. In various implementations described herein, transduction of cells may be carried out using viral vectors or non-viral vectors.

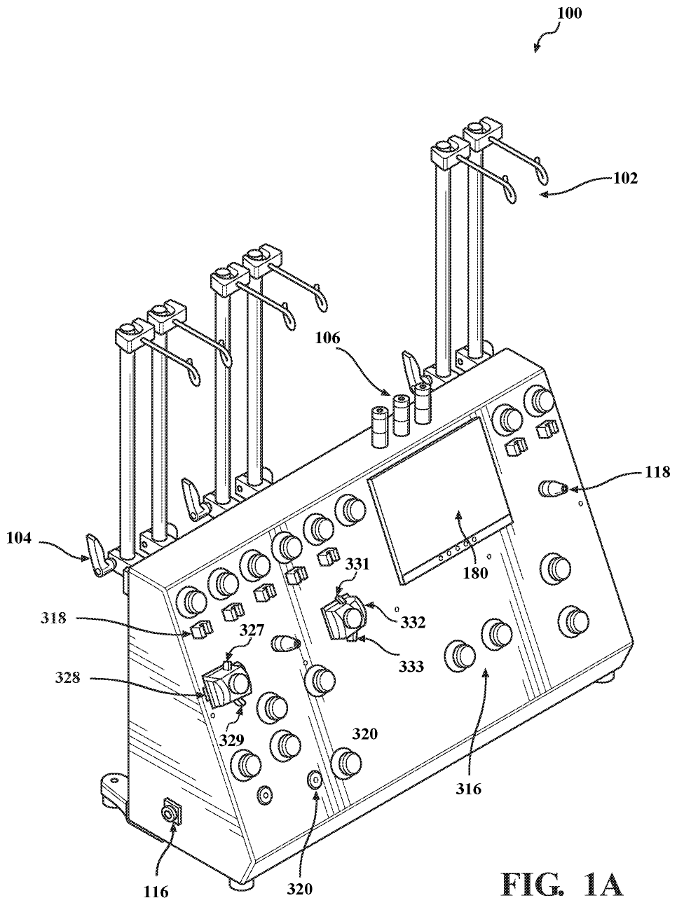

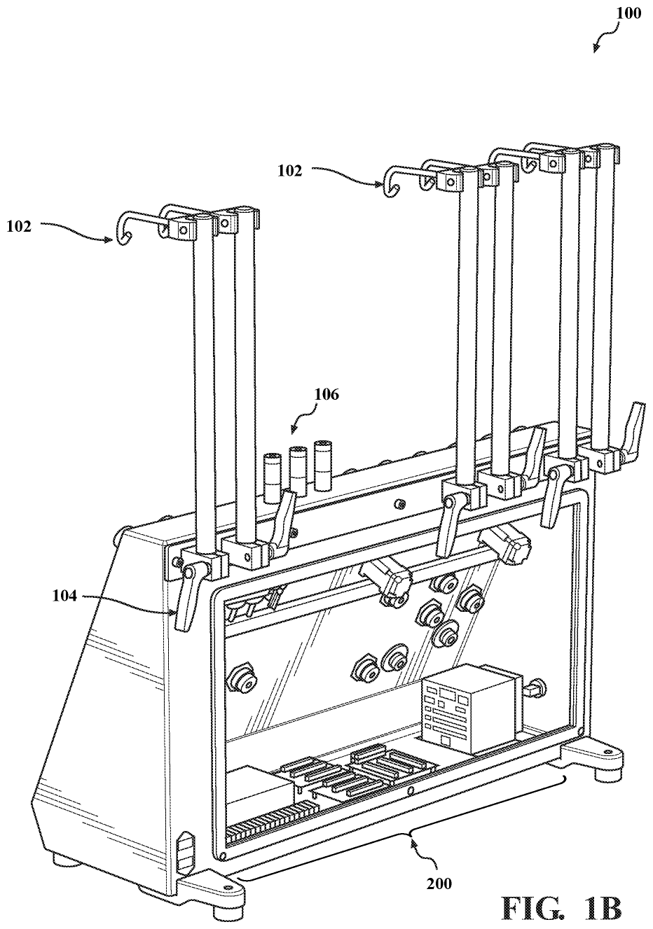

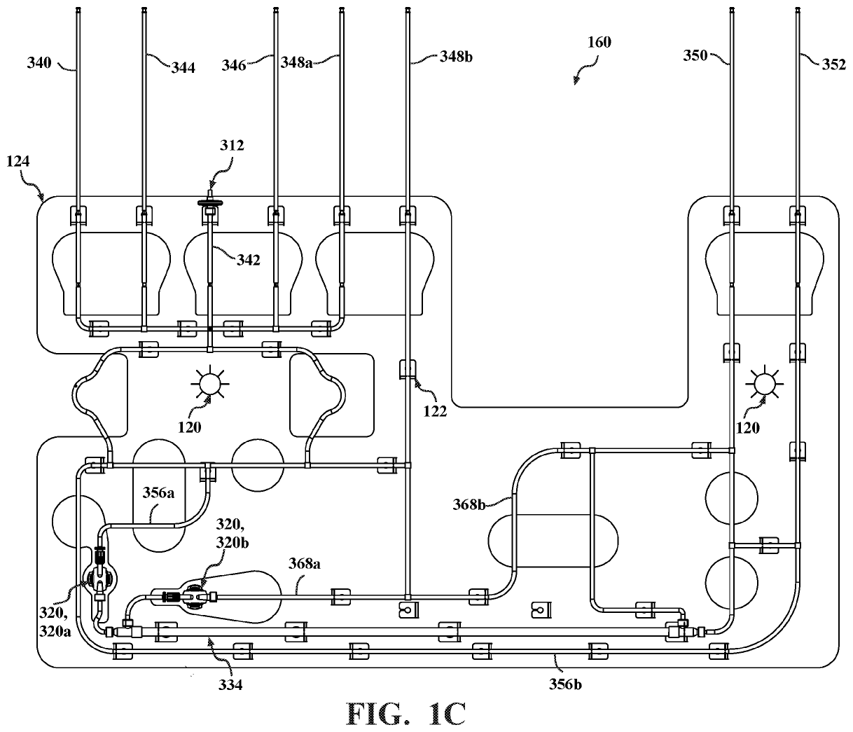

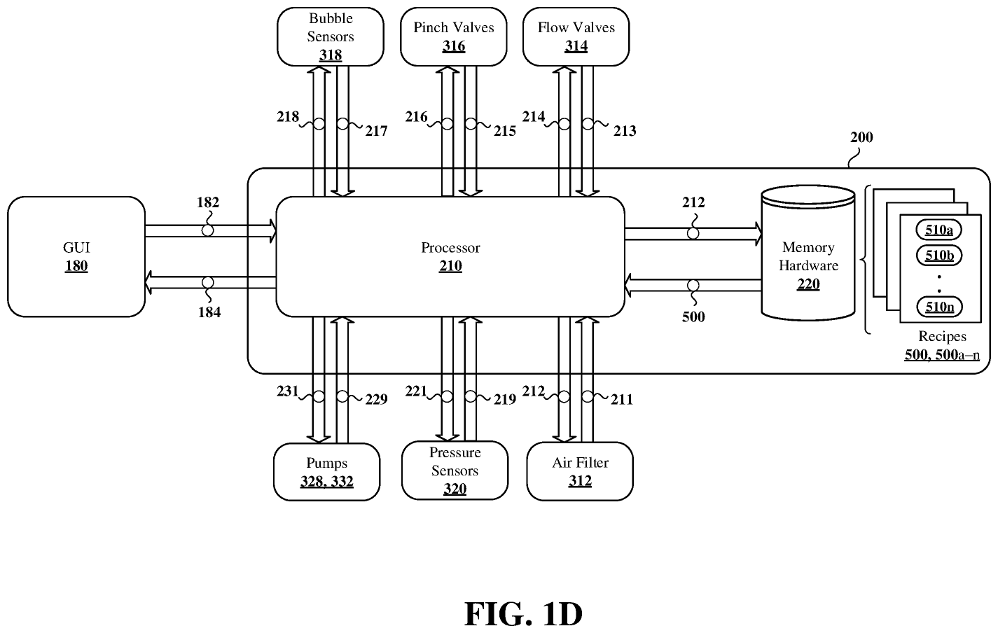

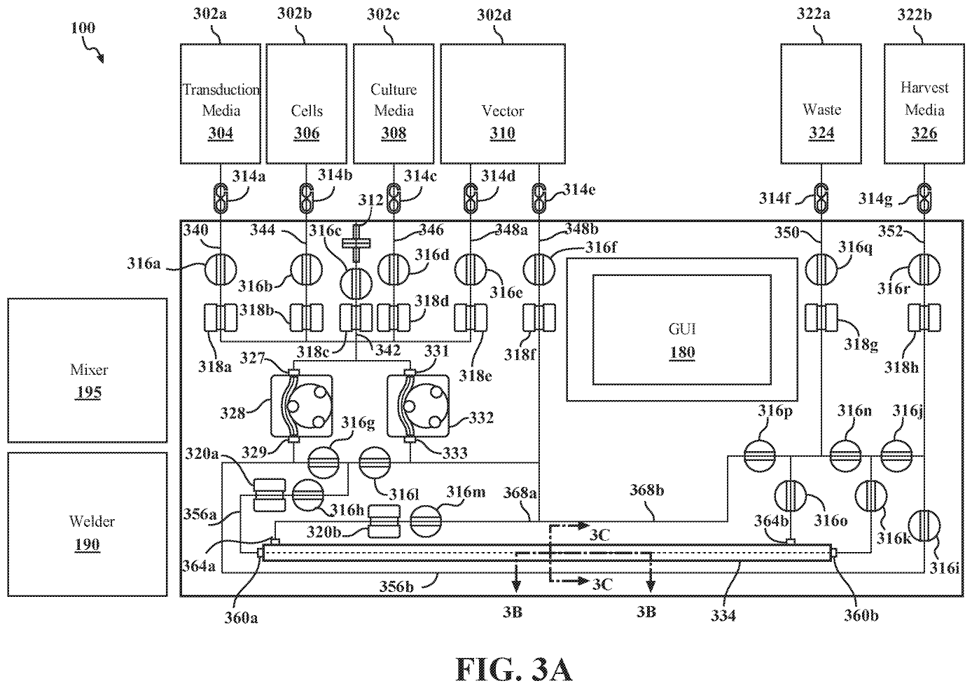

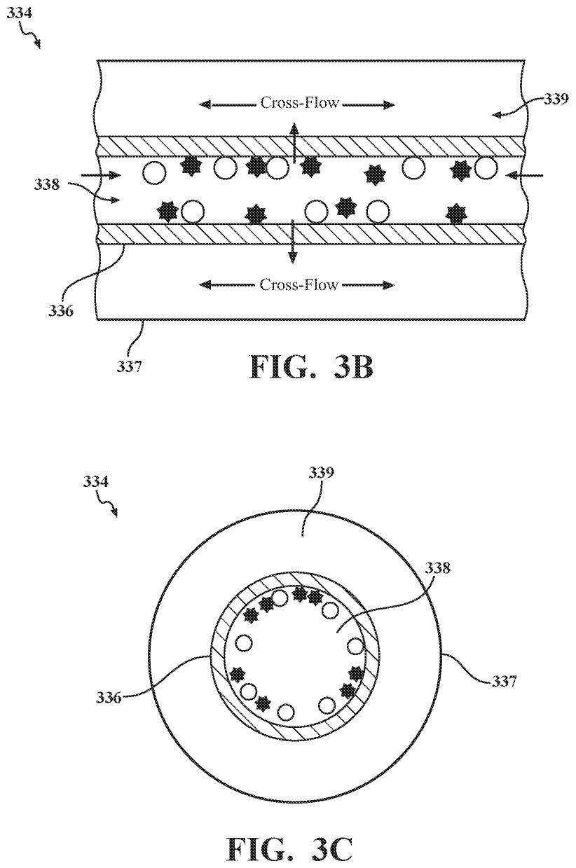

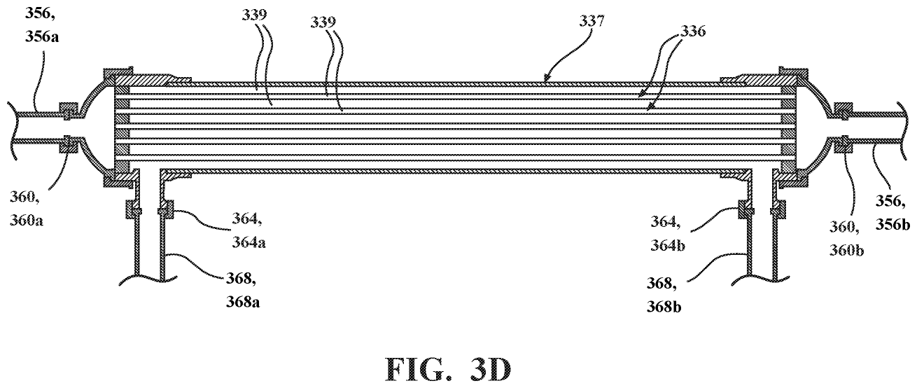

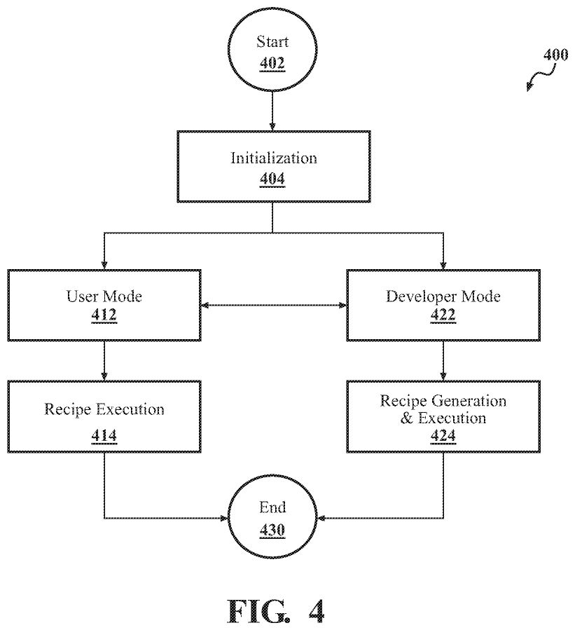

FIGS. 1A-1C illustrate perspective views of a fully automated hollow fiber cell transduction system 100. The fully automated hollow fiber cell transduction system 100 (also referred to herein as simply “hollow fiber system 100”) includes one or more bag hooks 102, hook adjustment handles 104, and status lights 106. Each bag hook 102 of the one or more bag hooks 102 is configured to support an input container 302 or output container 322 (FIG. 3). The hook adjustment handles 104 are configured to allow adjustment of the bag hooks 102 to raise or lower the input container 302 or output container 322 in the vertical direction. In some examples, the status lights 106 indicate to a user of the hollow fiber system 100 an operational status of the device. For example, the status lights 106 may include a green light that indicates to the user that a process is active, a blue light that indicates the process is complete, and a red light that indicates that user input is required.

The hollow fiber system 100 may also include one or more pumps 328, 332, bubble sensors 318, fluid sensors 320, and pinch valves 314 as described in greater detail with reference to FIG. 3. Referring now to FIG. 1A, a perspective view illustrates a first pump 328 including a first pump inlet 327 and a first pump outlet 329; a second pump 332 including a second pump inlet 331 and a second pump outlet 333; and the hollow fiber system 100 also including a filter module 334, described in more detail with respect to FIG. 3A. The hollow fiber system 100 is aseptically closed from the surrounding environment. That is, the hollow fiber system 100 is free from bacteria or viruses outside of the hollow fiber system 100 itself.

FIG. 1C illustrates an example tube set 160 disposed on a mounting card 124 that is configured to attach to the hollow fiber system 100. That is, post mounting holes 120 of the mounting card 124 attach to tube set mounting posts 118 (FIG. 1A) of the hollow fiber system 100. The tube set 160 also includes a plurality of conduits (e.g., transduction conduit 340, air filter conduit 342, cell conduit 344, culture conduit 346, first and second virus conduits 348a, 348b, waste conduit 350, harvest conduit 352, first and second intra-capillary conduits 356a, 356b, and first and second extra-capillary subconduits 368a, 368b) that fluidly couple the components of the hollow fiber system 100 to each other, as discussed in greater detail below with respect to FIG. 3A. The conduits may be PVC tubing and are adjustable to change fluid paths of the hollow fiber system 100. In particular, the conduits 340, 342, 344, 346, 348, 350, 352, 356, 368 attach to a plurality of tube holders 122 that allow a user to easily and quickly adjust fluid paths of the conduits. In some examples, the tube set 160 includes fluid sensors 320 (e.g., a first fluid sensor 320, 320a and a second fluid sensor 320, 320b), a filter module 334, and an air filter 312. In these examples, the fluid sensors 320 may be disposable fluid sensors. In some implementations, once cells 306 (as shown in FIG. 3A) are harvested via the harvest conduit 352, the cells are transferred to a suitable bioreactor or culture vessel for expansion. Transduced cells are then expanded for 3-20 days in a suitable culture medium and then washed and suspended in a final formulation buffer and cryopreserved in a suitable formulation for future therapeutic use.

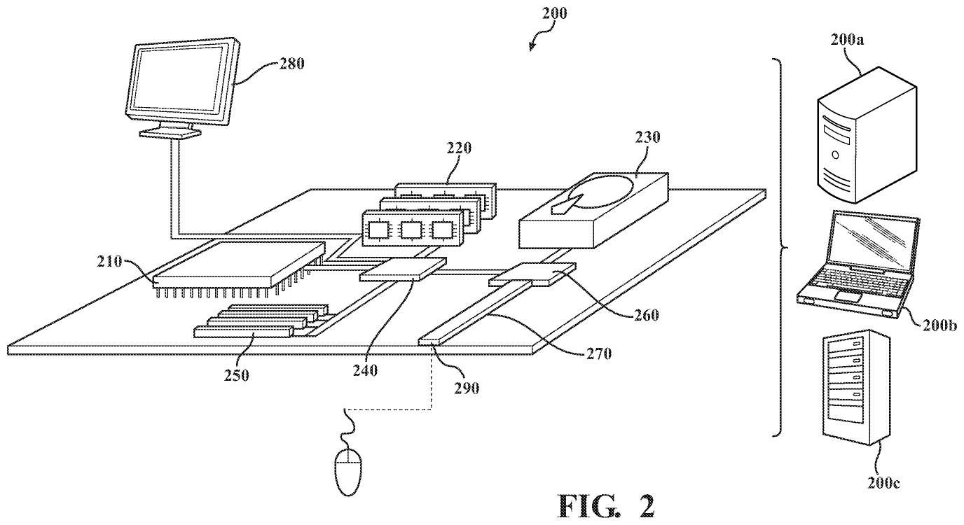

Optionally, the hollow fiber cell transduction system 100 may also include an Ethernet port 116, a graphical user interface (GUI) 180, and the tube set mounting posts 118. The Ethernet port 116 may communicatively couple the hollow fiber system 100 to a remote computing system that is configured to execute instructions at the hollow fiber system 100 remotely. The GUI 180 allows a user to provide user inputs to control operations at the hollow fiber system 100. The hollow fiber system 100 also includes a computing device 200 (FIG. 1B) that is in communication with each component of the hollow fiber system 100. The computing device (i.e., controller) 200 may include a processor (e.g., data processing hardware) 210 and memory (e.g., memory hardware) 220, discussed in more detail with reference to FIG. 2.

FIG. 1D illustrates electrical communication between the GUI 180, the processor 210, the memory hardware 220, and the components of the hollow fiber system 100. In the example shown, the GUI 180 sends a request 182 to the processor 210 to execute a recipe 500 from a plurality of recipes 500, 500a-n stored at the memory hardware 220 of the controller 200. Each recipe 500 of the plurality of recipes 500 stored at the memory hardware 220 includes a plurality of steps 510, 510a-n. Here, each step 510 instructs the processor 210 to obtain information from a component of the hollow fiber system 100 and/or instruct a component of the hollow fiber system 100. In response to receiving the request 182, the processor 210 sends a recipe retrieval request 212 to the memory hardware 220 to retrieve a recipe 500 corresponding to the recipe retrieval request 182.

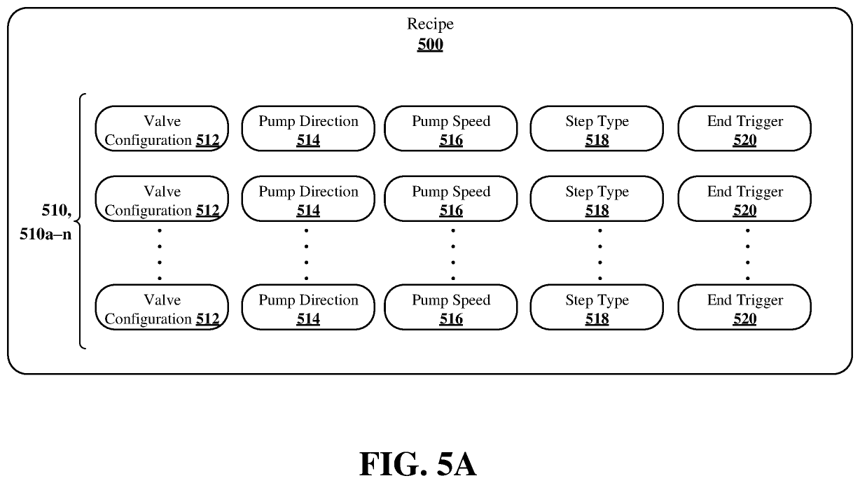

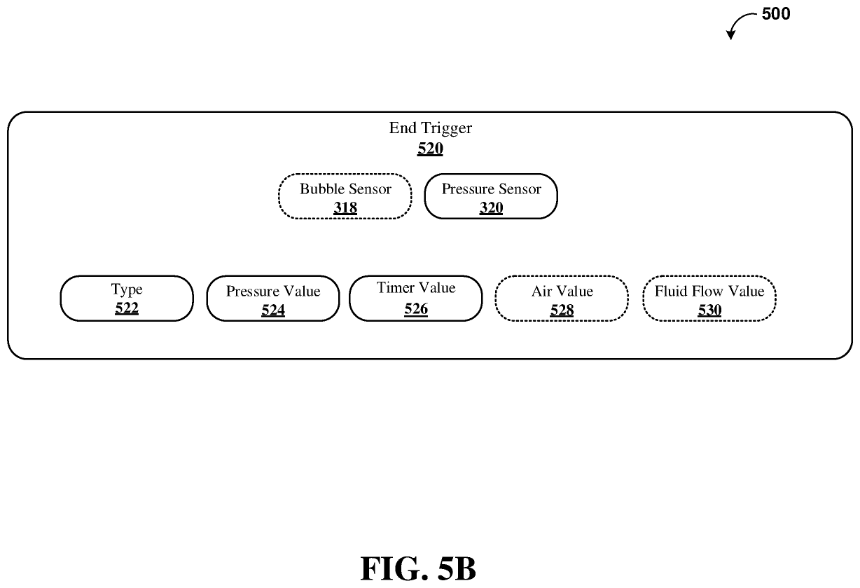

In some implementations, the controller 200 is in electrical communication with an air filter 312, flow valves 314, pinch valves 316, bubble sensors 318, fluid sensors 320, and pumps 328, 332. In the example shown, the processor 210 receives an air filter signal 211 from the air filter 312 and sends an air filter instruction signal 212 to the air filter 312. In this example, the processor 210 receives a flow valve signal 213 from the flow valves 314 and sends a flow valve instruction signal 214 to the flow valves 314. Here, the processor 210 also receives a pinch valve signal 215 from the pinch valves 316 and sends a pinch valve instruction signal 216 to the flow valves 314. Continuing with the example, the processor 210 receives a bubble sensor signal 217 from the bubble sensors 318 and sends a bubble sensor instruction signal 218 to the bubble sensors 318. The processor 210 also receives a fluid flow signal 219 from the fluid sensors 320 and sends a fluid flow instruction signal 221 to the fluid sensors 320. In some examples, the fluid sensors 320 are pressure sensors and the fluid flow signal 219 indicates a pressure value. In other examples, the fluid sensors 320 are fluid flow sensors and the fluid flow signal 219 indicates a volume of fluid flow through the fluid sensor 320 (e.g., liters/min). In this example, the processor 210 also receives a pump signal 229 from the pumps 328, 332 and sends a pump instruction signal 231 to the pumps 328, 332. The contents of the instruction signals are described in more detail with reference to FIGS. 5A and 5B.

In some examples, the processor 210 retrieves signals 211, 213, 215, 217, 219, 229 at a predetermined time interval. In other examples, the recipe 500 instructs the processor 210 when to retrieve the signals 211, 213, 215, 217, 219, 229. The processor 210 sends results 184 to the GUI 180 based on execution of the recipe 500 of the request 182. The GUI 180 may display the results 184 to the user and include information received from one of the components of the hollow fiber system 100. Thus, the hollow fiber system 100 is a closed system in that the controller 200 sends instruction signals 212, 214, 216, 218, 221, 231 based, at least in part, on the retrieved signals 211, 213, 215, 217, 229.

FIG. 2 is schematic view of the computing device (i.e., controller) 200 that may be used to implement the systems and methods described in this disclosure. The components shown here, their connections and relationships, and their functions, are meant to be exemplary only, and are not meant to limit implementations of the inventions described and/or claimed in this disclosure.

The computing device 200 includes a processor (e.g., data processing hardware) 210, memory (e.g., memory hardware) 220, a storage device 230, a high-speed interface/controller 240 connecting to the memory 220 and high-speed expansion ports 250, and a low speed interface/controller 260 connecting to a low speed bus 270 and a storage device 230. Each of the components 210, 220, 230, 240, 250, and 260, are interconnected using various busses, and may be mounted on a common motherboard or in other manners as appropriate. The processor 210 can process instructions (e.g., recipes) for execution within the computing device 200, including instructions stored in the memory 220 or on the storage device 230 to display graphical information for a graphical user interface (GUI) 180 on an external input/output device, such as display 280 coupled to high speed interface 240. In other implementations, multiple processors and/or multiple buses may be used, as appropriate, along with multiple memories and types of memory. Also, multiple computing devices 200 may be connected, with each device providing portions of the necessary operations (e.g., as a server bank, a group of blade servers, or a multi-processor system).

The memory 220 stores information non-transitorily within the computing device 200. The memory 220 may be a computer-readable medium, a volatile memory unit(s), or non-volatile memory unit(s). The non-transitory memory 220 may be physical devices used to store programs (e.g., sequences of instructions) or data (e.g., program state information) on a temporary or permanent basis for use by the computing device 200. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 230 is capable of providing mass storage for the computing device 200. In some implementations, the storage device 230 is a computer-readable medium. In various different implementations, the storage device 230 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described in this disclosure. The information carrier is a computer- or machine-readable medium, such as the memory 220, the storage device 230, or memory on processor 210.

The high speed controller 240 manages bandwidth-intensive operations for the computing device 200, while the low speed controller 260 manages lower bandwidth-intensive operations. Such allocation of duties are exemplary only. In some implementations, the high-speed controller 240 is coupled to the memory 220, the display 280 (e.g., through a graphics processor or accelerator), and to the high-speed expansion ports 250, which may accept various expansion cards (not shown). In some implementations, the low-speed controller 260 is coupled to the storage device 230 and a low-speed expansion port 290. The low-speed expansion port 290, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 200 may be implemented in a number of different forms, as shown in the figure. For example, it may be implemented as a standard server 200a or multiple times in a group of such servers 200a, as a laptop computer 200b, or as part of a rack server system 200c.

Various implementations of the systems and techniques described herein can be realized in digital electronic and/or optical circuitry, integrated circuitry, specifically designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

FIG. 3A and FIGS. 12-24 illustrate a schematic layout of the hollow fiber system 100 for performing automated viral vector transduction of cells. The hollow fiber system 100 includes one or more input containers 302, 302a-302d each containing an input material, one or more output containers 322, 322a-322b each configured to receive an output material, an intra-capillary pump (i.e., the first pump) 328, an extra-capillary pump (i.e., the second pump) 332, and the filter module 334 including one or more hollow fibers 336. The filter module 334 provides a convenient means for introducing various materials into the hollow fiber system 100 and retrovirus materials.

FIG. 3B illustrates a horizontal cross-section of a simplified example of a hollow fiber 336 of the filter module 334. The horizontal cross-section is a cross-section of the hollow fiber 336 taken along Line 3B-3B as shown in FIG. 3A. The hollow fiber 336 may be enclosed within a casing 337 and form an example of the filter module 334.

As shown, the space within the hollow fiber 336 defines an intra-capillary space 338 and the space outside of the hollow fiber 336 defines an extra-capillary space 339. For example, the extra-capillary space 339 is the space between the hollow fiber 336 and the casing 337. While the illustrated example shows a single hollow fiber 336 defining the intra-capillary space 338, it will be appreciated that there may be a plurality of the hollow fibers 336 arranged in parallel and cooperatively defining the intra-capillary space 338, such as in the example shown in FIG. 3D. That is, the filter module 334 may include any number of hollow fibers 336. One example of a filter module includes a MicroKros Hollow Fiber from REPLIGEN.

FIG. 3C illustrates a vertical cross-section of the hollow fiber 336 of the present disclosure. The vertical cross-section is a cross-section of the hollow fiber 136 taken along Line 3C-3C as shown in FIG. 3A. The vertical cross-section also illustrates the hollow fiber 336 disposed within the casing 337. The hollow fiber 336 includes a membrane with a plurality of pores defining a filter passage between the intra-capillary space 338 and the extra-capillary space 339. The porous membrane allows fluid to flow from the intra-capillary space 338 to the extra-capillary space 339 via the plurality of pores and vice-versa. As set forth above and shown in FIG. 3D, a plurality of the hollow fibers 336 may be implemented in the filter module 334 where all of the hollow fibers 136 are contained within the casing 337. Here, each hollow fiber 336 defines a discrete portion of the intra-capillary space 338. With continued reference to FIGS. 3A and 12-24, the filter module 334 includes a pair of intra-capillary ports 360 (e.g., a first intra-capillary port 360, 360a and a second intra-capillary port 360, 360b) that are fluidly coupled to the intra-capillary space 338 at opposite ends of the filter module 334 while a pair of extra-capillary ports (e.g., a first extra-capillary port 364, 364a and a second extra-capillary port 364, 364b) are fluidly coupled to the extra-capillary space 339 at opposite ends of the filter module 334.

In some examples, the one or more input containers 302 includes a transduction container 302, 302a that contains transduction media 304, and is fluidly coupled with the first pump 328 and the second pump 332 through a transduction conduit 340. In these examples, the one or more input containers 302 may also include a cell input container (e.g., cell source) 302, 302b that contains a population of cells 306 and is fluidly coupled with the first pump 328 and the second pump 332 through a cell conduit 344.

The population of cells 306 (interchangeably referred to as simply “cells 306”) may include immune cells or stem cells, e.g., B-cells, αβT cells, γδ T cells, NK (natural killer) cells, iPSCs, iPSC derived NK cells, hematopoietic stem cells, monocytes, other lymphoid cells or progenitor cells, suitable for use in cell therapies. In some implementations, a vector includes a chimeric antigen receptor (CAR) construct which is expressed on the surface of a transduced cell following transduction using the systems and/or methods according to this disclosure.

In some examples, once harvested, transduced cells 306 are separated from untransduced cells 306 and vector 310 by using suitable means in the art, e.g., affinity isolation of the transduced cells 306 from vector 310 and untransduced cells 306 using an antibody that binds the CAR being expressed on the cells 306 of the transduced cells 306 or use of flow cytometry. Other suitable means in the art that may be used include, but are not limited to, size exclusion separation or some other method such as use of a column, membrane, etc. to separate cells 306 from vector 310. Once cells 306 are isolated, separated, or removed following the harvest step, cells 306 may either be expanded and then cryopreserved or be cryopreserved following the harvest step and the cryopreserved cells 306 may be subsequently used for future therapeutic use.

The one or more input containers 302 may also include a culture container 302, 302c that contains culture media 308 and is fluidly coupled with the first pump 328 and the second pump 332 through a culture conduit 346. Optionally, the hollow fiber system 100 may include an air filter 312 that is fluidly coupled to the first pump 328 and the second pump 332 through an air filter conduit 342. The one or more input containers 302 also include a vector container (e.g., viral or non-viral vector source) 302, 302d that contains viruses or a vector 310, and is fluidly coupled with the first pump 328 and the second pump 332 through a first vector conduit 348, 348a and a second vector conduit 348, 348b.

The vector 310 may include virus or non-viral particles suitable for transduction of cells. In some implementations, the vector 310 includes virus containing a cargo to be delivered to a cell. In some implementations, such a cargo includes a CAR construct which is expressed on the surface of cells following transduction by the viral vector. The viruses may be derived from a lentivirus, a retrovirus, an adenovirus, an adeno-associated virus, or a hybrid virus. In some examples, the vector 310 includes a non-viral vector instead of viruses. Here, the non-viral vectors may include liposomes, lipid particles, carbon, non-reactive metals, gelatin, polyamine nanospheres, and/or inorganic nanoparticles. Additional examples of non-viral vectors include, for example spheroplasts, red blood cell ghosts, colloidal metals, inorganic nanoparticles, DEAE Dextran plasmids, among others, or a combination thereof. In some implementations, inorganic nanoparticles are calcium phosphate nanoparticles. The vector 310 may include non-viral vectors, viral vectors, among others, and any combination thereof.

In the example shown, the first pump 328 includes the first pump inlet 327 in fluid communication with each input container 302 of the plurality of input containers 302 and the air filter 312 via a corresponding conduit 340, 342, 344, 346, 348. In these examples, the second pump 332 may include the second pump inlet 331 also in fluid communication with each input container 302 of the plurality of input containers 302 and the air filter 312 via a corresponding conduit 340, 342, 344, 346, 348. In some scenarios, it may be desirable to only have a subset of the transduction media 304, cells 306, culture media 308, and the virus 310 (collectively referred to as input materials 304, 306, 308, 310) in fluid communication with the first pump 328 and/or second pump 332. As such, the hollow fiber system 100 includes a plurality of flow valves 314, 314a-n and a plurality of pinch valves 316, 316a-n to selectively permit fluid flow from the input containers 302 to the first pump 328 and/or the second pump 332.

The flow valves 314 may include a manual pinch valve that require user input to open or close the flow valve 314. As used herein, a flow valve 314 or pinch valve 316 in an “open” state allows fluid to flow through the respective valve while a flow valve 314 or pinch valve 316 in a “closed” state does not allow fluid to flow through the respective valve. In some examples, the flow valve 314 is in electrical communication with the controller 200 such that the controller 200 can instruct the flow valve 314 to open or close without user input. In the open state, the flow valve 314 allows the contents of an input container 302 or an output container 322 to be loaded into the hollow fiber system 100. Conversely, in the closed state, the flow valve 314 does not allow an input container 302 or an output container to be loaded into the hollow fiber system 100.

The pinch valves 316 may be in electrical communication with the controller 200 such that the controller 200 can instruct the pinch valves 316 to open or close according to a recipe, described in more detail below. That is, the controller 200 may instruct each pinch valve 316 of the plurality of pinch valves 316 to open or close independently from the other pinch valves 316. Accordingly, by instructing the plurality of pinch valves 316 to open or close, the controller 200 can define fluid paths for the input material 304, 306, 308, 310 to flow. In some examples, the hollow fiber system 100 also includes a plurality of bubble sensors 318 that detect the presence of bubbles (e.g., air) in the input materials 304, 306, 308, 310 and help ensure that the filter module 334 receives bubble-free input materials 304, 306, 308, 310. Thus, the bubble sensors 318 are in electrical communication with the controller 200 to provide an air quantity value that indicates the presences of bubbles.

In the example shown, each input container 302 of the plurality of input containers 302 is associated with respective ones of a flow valve 314, a pinch valve 316, and a bubble sensor 318 disposed along the corresponding conduit 340, 342, 344, 346, 348 between the first pump inlet 327 and the respective input container 302. Moreover, each input container 302 of the plurality of input containers 302 is associated with a flow valve 314, a pinch valve 316, and a bubble sensor 318 disposed at conduit 340, 342, 344, 346, 348 between the second pump inlet 331 and the respective input container 302. In particular, the pinch valve 316 is disposed between the flow valve 314 and the bubble sensor 318 such that the pinch valve 316 is downstream of the flow valve 314 and upstream of the bubble sensor 318 with respect to a direction of flow from each input container 302.

Depending on the flow valve 314 configuration and/or pinch valve 316 configuration, the first pump inlet 327 of the first pump 328 receives one or more of the input materials 304, 306, 308, 310, and supplies the input material 304, 306, 308, 310 at a desired rate to the pair of intra-capillary ports 360 of the filter module 334. In the example shown, the first pump outlet 329 of the first pump 328 is in selective fluid communication with the pair of intra-capillary ports 360. That is, the first pump outlet 329 is fluidly coupled to the first intra-capillary port 360a through a first intra-capillary conduit 356, 356a and is fluidly coupled to the second intra-capillary port 360b through a second intra-capillary conduit 356, 356b. In particular, pinch valves 316g, 316h control fluid flow from the first pump outlet 329 to the first intra-capillary port 360a and pinch valves 316i, 316j, 316k control fluid flow from the first pump outlet 329 to the second intra-capillary port 360b.

Continuing with the example, depending on the flow valve 314 configuration and/or pinch valve 316 configuration, the second pump inlet 331 of the second pump 332 receives one or more of the input materials 304, 306, 308, 310, and supplies them at a desired rate to the pair of extra-capillary ports 364 of the filter module 334. That is, the second pump outlet 333 is fluidly coupled to the first extra-capillary port 364a through a first extra-capillary subconduit 368, 368a and is fluidly coupled to the second extra-capillary port 364b through a second extra-capillary subconduit 368, 368b. In particular, pinch valve 316m controls fluid flow from the second pump outlet 333 to the first extra-capillary port 364a and pinch valves 316p, 316o control fluid flow from the second pump outlet to the second extra-capillary port 364b.

In some examples, a first fluid sensor 320a is disposed between the first pump outlet 329 and the first intra-capillary port 360a and a second fluid sensor 320b is disposed between the second pump outlets 333 and the first extra-capillary port 364a. The fluid sensors 320 are configured to ensure that the first pump 328 and the second pump 332 provide accurate pressure to the first intra-capillary port 360a and the first extra-capillary port 364a respectively. Thus, the fluid sensors 320 are in electrical communication with the controller 200 and provide measured pressure values at the first intra-capillary port 360a and the first extra-capillary port 364a to the controller 200.

In some examples, the one or more output containers 322 include a waste container 322, 322a that contains waste 324, and is fluidly coupled with the second intra-capillary port 360b and the second extra-capillary port 364b through a waste conduit 350. In these examples, the waste container 322a is also fluidly coupled with the first extra-capillary port 364a through the first extra-capillary subconduit 368a and the waste conduit 350. In some implementations, the one or more output containers 322 may also include a harvest container 322, 322b that contains harvest media 326, and is fluidly coupled with the second intra-capillary port 360b and the second extra-capillary port 364b through a harvest conduit 352. In these implementations, the harvest container 322b is also fluidly coupled with the first extra-capillary port 364a through the first extra-capillary subconduit 368a and the harvest conduit 352.

In the example shown, each output container 322 of the plurality of output containers 322 is associated with respective ones of a flow valve 314, a pinch valve 316, and bubble sensor 318 disposed at along the corresponding conduit 350, 352 between the second intra-capillary port 360b and the second extra-capillary port 364b. In particular, the pinch valve 316 is disposed between the flow valve 314 and the bubble sensor such that the pinch valve 316 is upstream of the flow valve 314 and downstream of the bubble sensor 318 with respect to a direction of flow into each output container 322. Continuing with the example, the waste container 322a is associated with pinch valves 316k, 316n disposed between the waste container 322a and the second intra-capillary port 360b and pinch valve 316o disposed between the waste container 322a and the second extra-capillary port 364b. Moreover, the harvest container 322b is associated with pinch valves 316j, 316k, 316r disposed between the waste container 322a and the second intra-capillary port 360b and pinch valve 316j, 316n, 316o, 316r disposed between the waste container 322a and the second extra-capillary port 364b. Thus, depending on the flow valve 314 configuration and/or pinch valve 316 configuration, output containers 322 receive fluid flow from the filter module 334.

The GUI 180 is configured to receive requests from a user for operating one of the components of the hollow fiber system 100. Accordingly, the GUI 180 and/or controller 200 is in operative communication with each component of the hollow fiber system 100. The request may include an instruction to operate the first pump 328 and the second pump 332 according to a recipe that includes one or more steps. For example, the GUI 180 may instruct the first pump 328 and the second pump 332 to operate according to a recipe. In another example, the GUI 180 may instruct the plurality of pinch valves 316 to open or close according to a recipe. In yet another example, the GUI 180 is in operative communication with the fluid sensors 320 and the bubble sensors 318 such that the GUI 180 displays values to the user that the GUI 180 receives from the fluid sensors 320 and the bubble sensors 318. In some instances, the GUI 180 is configured to display the recipe to the user from memory hardware 220 responsive to receiving the request from the user.

FIG. 4 illustrates an example arrangement of operations for a method 400 of operating the GUI 180 in a user mode and a developer mode. The method 400 starts at operation 402 and enters an initialization routine at operation 404. The initialization routine 404 is configured to perform a basic check of components and connections of the hollow fiber system 100. For example, the initialization routine may determine whether the pinch valves 316, the first and second pumps 328, 332, the bubble sensors 318, and the fluid sensors 320 are in a default state and/or operational. The initialization routine may also ensure that the controller 200 and/or the GUI 180 can communicate with all components of the hollow fiber system 100. The initialization routine must successfully complete before proceeding to operation 412 (i.e., user mode) or operation 422 (i.e., developer mode).

After the initialization routine 404 completes successfully, the method 400 may proceed to either operation 412 or operation 422. That is, the GUI 180 may receive a user input requesting to operate the hollow fiber system 100 in user mode and proceed to operation 412. While in user mode the user may select one or more recipes to be executed by the hollow fiber system 100. Upon selection of a recipe by the user, the method 400 proceeds to operation 414 to execute the selected recipe. The execution of recipes is described in more detail below, however, while in the user mode the GUI 180 limits the user to only executing recipes stored at the memory hardware 220. That is, in user mode the user may be limited to executing a recipe from a plurality of predefined recipes stored at the memory hardware 220. The GUI 180 may display information about the recipe to the user during execution of a recipe. After completing execution of the recipe, the method 400 terminates at operation 430.

Alternatively, after the initialization routine 404, the GUI 180 may receive a user input requesting to operate the hollow fiber system 100 in developer mode and proceed to operation 422. While in developer mode, the user may generate or execute the method 400 proceeds to operation 424 to allow the user to generate and/or execute a recipe. Accordingly, in developer mode, the GUI 180 may prompt the user to enter credentials verifying that the user has permission to operate the GUI in developer mode. After generating the new recipe or executing the recipe, the method terminates at operation 430.

Referring back to FIG. 3A and FIGS. 12-24, in some implementations, the hollow fiber system 100 includes a mixer 195 configured to mix one or more of the input materials 304, 306, 308, 310 together before loading the material into the filter module 334. In some instances, pre-mixing the input materials increases the interaction of the one or more input materials 304, 306, 308, 310 during transduction. For example, the mixer 195 may mix the cells 306 with the virus (i.e., vector) 310 to generate a mixture thereof before loading the cells 306, the virus 310, or the mixture into the respective input container 302. In these examples, pre-mixing the cells 306 and the virus 310 before transduction increases interactions (e.g., collisions) between the cells 306 and the virus 310 during transduction. In the illustrated example, the mixer 195 is shown as a peripheral device that operates in parallel with the hollow fiber system 100, whereby the inputs can pre-mixed and then provided in one of the input containers. Alternatively, the hollow fiber system 100 may include one or more of the mixers 195 integrated between the input containers 302 and the one or both of the pumps 328, 332 such that the input materials 304, 306, 308, 310 can be mixed with each other prior to dispensing to the filter module 334. Optionally, the mixer 195 may include a conduit that fluidly couples the mixer 195 to one or more conduits of the hollow fiber system 100 or one of the input containers 302.

In some implementations, the hollow fiber system 100 includes a welder 190 configured to weld an input container 302 or output container 322 to a corresponding conduit 340, 342, 344, 346, 348, 350, 352. That is, the one or more input containers 302 and the one or more output containers 322 may be removed from the hollow fiber system 100 or attached to the hollow fiber system 100 by the welder 190. For example, when loading the transduction container 302a into the hollow fiber system 100, the welder 190 may weld the transduction container 302a to the transduction conduit 340. Thus, the welder 190 provides a sealed connection between transduction container 302a and the hollow fiber system 100.

Implementations herein are further directed towards a computer-implemented method that when executed on the data processing hardware 210 of the controller 200, causes the data processing hardware 210 to perform operations at the hollow fiber system 100. In particular, the controller 200 may store a plurality of recipes 500 at memory hardware 220. Referring now to FIG. 5A, each recipe 500 includes a plurality of steps 510, 510a-n wherein each step 510 of the recipe 500 instructs the hollow fiber system 100 to perform an operation. In some implementations, the recipe 500 is a CSV file or any other table of data wherein each row of data corresponds to a step 510 of the recipe 500 and each column of data corresponds to a component instruction (e.g., valve configuration, pump speed, etc.) for the respective step. Thus, the controller 200 iterates through each step 510 of the recipe 500 until a last step of the recipe 500 completes execution. Notably, the recipe 500 automates control of the hollow fiber system 100 thereby allowing a user to perform cell transduction with little or no user interaction with the hollow fiber system 100.

In the example shown, each step 510 of the recipe 500 includes at least one of a valve configuration state 512, a pump direction state 514, a pump speed state 516, a step type 518, and an end trigger threshold 520. Thus, at each step 510 of the recipe 500, the controller 200 instructs the hollow fiber system 100 according to at least one of the valve configuration state 512, the pump direction state 514, the pump speed state 516, the step type 518, and the end trigger threshold 520 (e.g., via one or more instruction signals 212, 214, 216, 218, 221, 231 as shown in FIG. 1D). Thus, in some examples, the controller 200 may instruct the hollow fiber system 100 according to the valve configuration state 512 and the pump speed 516 at a single step of the recipe 500. The valve configuration state 512 may specify to the controller 200 whether to open or close a flow valve 314 and/or a pinch valve 316 via the flow valve instruction signal 214 and the pinch valve instruction signal 216 (FIG. 1D) respectively. In some examples, the valve configuration state 512 specifies whether the controller 200 opens all the pinch valves 316 or closes all the pinch valves 316. In other examples, the valve configuration state 512 specifies an independent valve state for each pinch valve 316 in the plurality of pinch valves 316. For example, a first pinch valve 316 may be open and a second pinch valve 316 may be closed. In some instances, the valve configuration state 512 specifies whether the controller 200 opens or closes each flow valve 314 of the plurality of flow valves 314. The controller 200 may determine whether each flow valve 314 and each pinch valve 316 is open or closed based upon receiving the flow valve signal 213 and the pinch valve instruction signal 216 (FIG. 1D) respectively.

The pump direction state 514 instructs a fluid flow direction for the pumps 328, 332 via the pump instruction signal 231 (FIG. 1D). For example, the pump direction state 514 may specify whether fluid flows in either a first direction (e.g., towards the output containers 322) or a second direction (e.g., towards the input containers 302). In some examples, the pump direction state 514 instructs the controller 200 to operate the first pump 328 in the first direction and operate the second pump 332 in the second direction. In other examples, the pump direction state 514 instructs the controller to operate both of the first pump 328 and the second pump 332 in the first direction or the second direction. In some implementations, the pump direction state 514 alternates for each step 510 of the recipe 500. For example, at a first step 510a of the recipe 500 the pumps 328, 332 may operate in the first direction and at a second step 510b of the recipe the pumps 328, 332 may alternate fluid flow direction and operate in the second direction. The controller 200 may determine the current pump direction state 514 of the first pump 328 and the second pump 332 based upon receiving the pump signal 229 from the pumps 328, 332 (FIG. 1D).

The pump speed 516 instructs the controller 200 to operate the pumps 328, 332 at a specified speed such as revolutions per minute (RPM) via the pump instruction signal 231 (FIG. 1D). The pump speed 516 may directly correlate with a fluid flow rate or pressure that the pumps 328, 332 generate. That is, operating the pumps 328, 332 at a faster speed increases the fluid flow rate or the pressure and operating the pumps 328, 332 at a slower speed decreases the fluid flow rate or the pressure. Thus, the controller may determine the pump speed 516 based on a target pressure or fluid flow parameter. In some examples, a pump speed 516 of ‘0’ indicates that the pumps 328, 332 are not currently pumping fluid. In these examples, the first pump 328 may be pumping fluid while the second pump 332 is not pumping fluid, or vice-versa. The controller 200 may determine the current pump speed state 516 of the first pump 328 and the second pump 332 based upon receiving the pump signal 229 from the pumps 328, 332 (FIG. 1D).

The step type 518 specifies a characteristic of an end trigger threshold 520 for the respective step 510 of the recipe 500. That is, the characteristics of the step type 518 represent what conditions must be met for the step 510 to complete. For example, a step type 518 may include characteristics of a fluid detection, a timer, an air detection, a fluid to air detection, an air to fluid detection, or a pressure detection. That is, the fluid detection and air detection step type 518 specify that the step 510 of the recipe 500 executes until a certain fluid detection parameter or air detection parameter is satisfied respectively (e.g., a target fluid detection parameter or a target air detection parameter). The timer specifies that the step 510 executes for a predetermined amount of time.

FIG. 5B illustrates an example end trigger threshold 520 for a step 510 of the recipe 500. The end trigger threshold 520 specifies certain conditions that, if met, either completes execution of the step 510 or terminates execution of the step 510 and/or the recipe 500. In some examples, one or more steps 510 of the recipe 500 include multiple end trigger thresholds 520. In particular, a first end trigger threshold 520 may indicate certain operational conditions that, if met, completes execution of the step 510. That is, when the hollow fiber system 100 meets conditions of the first end trigger threshold 520 for a step 510, the controller 200 completes execution of the step 510 successfully. The controller 200 may determine whether the hollow fiber system 100 meets the conditions of the end trigger thresholds 520 using the one or more received signals 211, 213, 215, 217, 219, 229 (as shown in FIG. 1D). For example, a step 510 may include a first end trigger threshold 520 to build 15 PSI of pressure. Here, the first end trigger threshold 520 represents a target value for the step 510 to achieve so that the hollow fiber system 100 operates properly. In this example, when the hollow fiber system 100 meets the conditions of the first end trigger threshold 520 (e.g., meets 15 PSI of pressure), the controller 200 successfully completes execution of the step 510 and proceeds to the next step 510 of the recipe 500.

Continuing with the example above, the step 510 of the recipe 500 may include a second end trigger threshold 520 that indicate certain different conditions that, if met, terminate execution of the step 510. As such, when the hollow fiber system 100 exceeds conditions of the second end trigger threshold 520 for a step 510, the controller 200 terminates execution of the step 510 and/or the entire recipe 500. For example, a step 510 may include a second end trigger threshold 520 to not exceed 50 PSI of pressure (e.g., a burst pressure). Here, the second end trigger threshold 520 represents a failsafe value that the hollow fiber system 100 should not exceed. In this example, when the hollow fiber system 100 exceeds the second end trigger threshold 520 (e.g., exceeds 50 PSI of pressure), the controller 200 either terminates execution of the step 510 or terminates execution of the entire recipe 500. In some examples, the second end trigger threshold 520 indicates a minimum value that the hollow fiber system 100 should not fall below (e.g., minimum of 10 PSI). In still other examples, a step 510 in a recipe 500 includes a second end trigger threshold 520 as described above. Alternatively, a second end trigger threshold 520 is included in a process that runs in the background while the hollow fiber system 100 is operating.

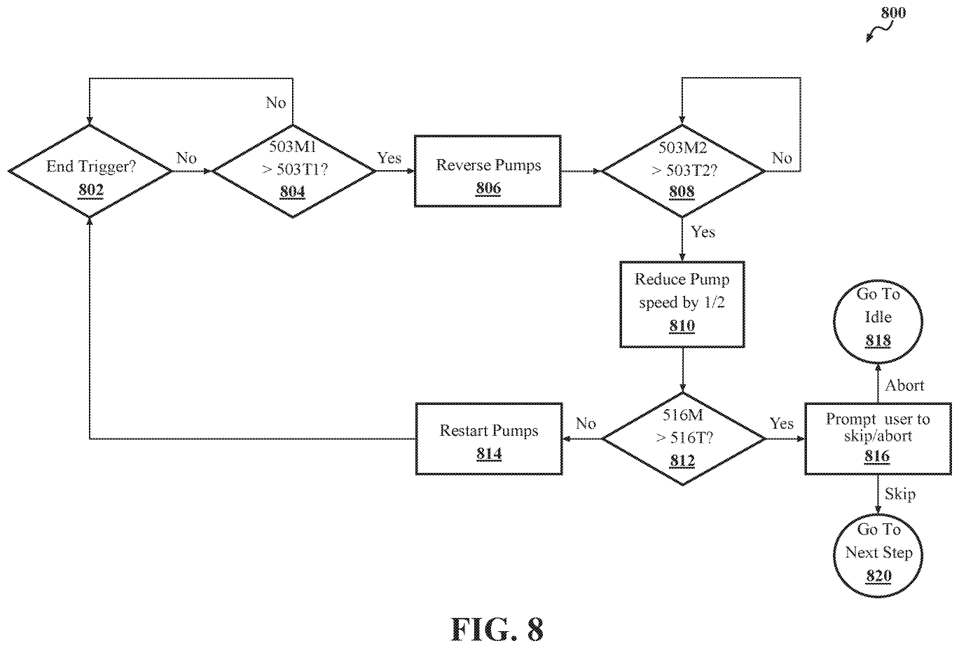

The controller 200 may either terminate the recipe 500, terminate the step 510, initiate a pump rate adjustment procedure, and/or prompt the user via the GUI 180 in response to the hollow fiber system meeting conditions for the second end trigger threshold 520. Terminating the recipe 500 or the step 510 may not be desirable because it may stop the cell transduction process. Accordingly, the controller 200 may execute the pump rate adjustment procedure when the hollow fiber system 100 is within a threshold value (e.g., ten percent) of the second end trigger threshold 520. The pump rate adjustment procedure is described in greater detail in reference to FIG. 8. Notably, by initiating the pump rate adjustment procedure, the controller 200 may prevent the hollow fiber system 100 from meeting the conditions of the second end trigger threshold value 520 such that the recipe 500 can continue execution without any errors. In some instances, the controller 200 prompts the user of the hollow fiber system 100 via the GUI 180. The prompt may allow the user to provide user input at the GUI 180 instructing the controller 200 to skip (i.e., bypass) the step 510, retry execution of the step 510, modify the end trigger threshold value 520, and/or allow the user to provide additional user inputs to complete execution of the recipe 500.

In some implementations, a step 510 of the recipe includes a first end trigger threshold 520 that indicates to the controller 200 what conditions must be met to complete the respective step 510 and a second end trigger threshold 520 that indicates to the controller 200 what conditions must be met to terminate the step 510. For example, the first end trigger threshold 520 indicates a target pressure of 25 PSI and the second end trigger threshold 520 indicates a minimum pressure of 5 PSI. In this example, when the hollow fiber system 100 reaches the target pressure of 25 PSI, the controller 200 completes execution of the step 510 successfully. Continuing with the example, when the hollow fiber system 100 falls below the minimum pressure of 5 PSI, the controller 200 terminates execution of the step 510. In another example, the first end trigger threshold 520 may indicate a target pump runtime of five minutes and the second end trigger threshold indicates a maximum pressure of 50 PSI. Here, when the pumps 328, 332 once the pumps 328, 332 run for five minutes, the controller 200 completes execution of the step 510 successfully. In parallel, the controller 200 also monitors the pressure of the hollow fiber system 100, and if the pressure exceeds 50 PSI, the controller 200 terminates execution of the step 510.

The end trigger threshold 520 indicates at least one of an end trigger type 522, a pressure value 524, a stopwatch timer value 526, an air quantity value 528 (e.g., bubble detection) or a fluid flow value (e.g., fluid flow rate, direction, or other fluid flow parameter) 530. Here, the controller 200 determines the pressure value 524 based upon the fluid flow signal 219 (FIG. 1D) received from one of the fluid sensors 320. Moreover, the controller 200 determines the air quantity value 528 using the bubble sensor signal 217 (FIG. 1d) received from one of the bubble sensors 318. In some examples, the controller 200 determines the fluid flow value 530 from the fluid flow signal 219 (FIG. 1D) received from one of the fluid sensors 320 (e.g., fluid flow sensors).

In other examples, the controller 200 determines the fluid flow value 530 using the pump speed 516 of the pumps 328, 332. In yet other examples, the controller 200 determines the fluid flow value 530 using some combination of the pump speed 516 and the fluid flow signal 219 (FIG. 1D). Optionally, the end trigger threshold 520 includes a sensor identification that identifies which sensor is associated with the end trigger threshold 520 (e.g., which sensor to obtain a signal from). For example, the end trigger threshold 520 illustrated in FIG. 5B identifies one of the bubble sensors 318 of the plurality of bubble sensors 318 to read an air quantity value 528 from and/or one of the fluid sensors 320 to read a pressure value 524 from.

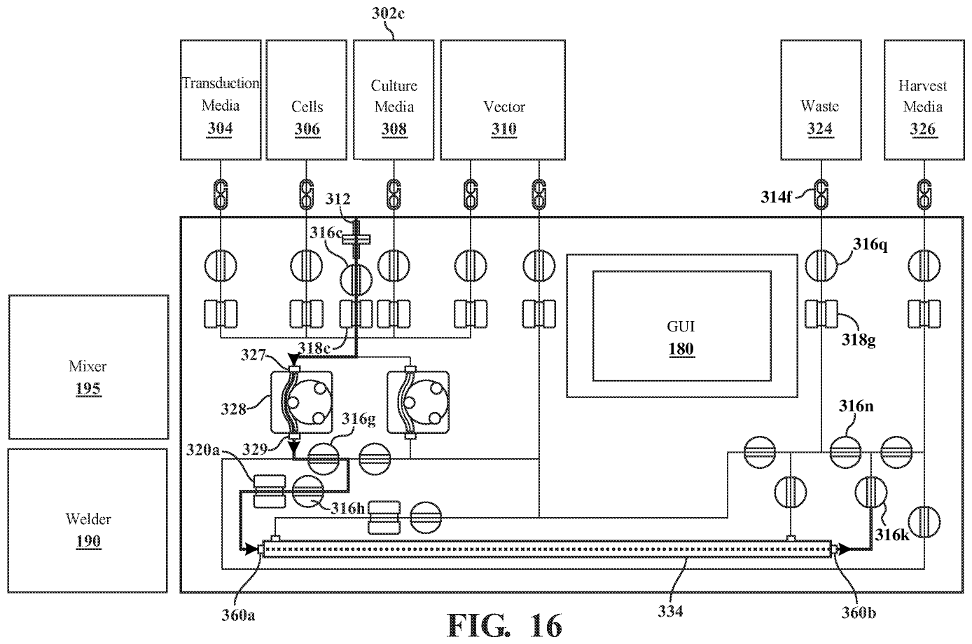

The end trigger type 522 may specify a type of end trigger threshold 520. The end trigger type 522 includes timers, pressure rise, pressure drop, fluid-to-air ratio, or integrity. The integrity end trigger type 522 refers to a pressure hold of the hollow fiber system 100. Here, the hollow fiber system 100 holds pressure at a specified value and measures the pressure drop over a period of time that indicates an integrity of the hollow fiber system (e.g., whether any leaks are present), discussed in greater detail in reference to FIG. 16. The end trigger type 522 may instruct the controller 200 to obtain certain values from the hollow fiber system 100. The pressure value 524 specifies a minimum and/or maximum pressure value. For example, the pressure value 524 specifies a maximum value of 30 pounds per square inch (PSI) and minimum value of 10 PSI.

The timer value 526 may specify a maximum or minimum execution time of a step 510 of a recipe 500. For example, a step 510 that has an average execution of ten seconds may have a timer value 526 (e.g., a watchdog timeout value) of sixty seconds. As such, if execution time of the step 510 exceeds sixty seconds, the controller 200 may determine there is an error with execution of the step 510. For example, a first end trigger threshold 520 may indicate a target pressure for the hollow fiber system 100 and a second end trigger threshold 520 indicates watchdog timer value 526 (e.g., one minute). Here, the watchdog timer value 526 indicates that if the hollow fiber system 100 does not achieve the target pressure of the first end trigger threshold 520 within one minute, then there must be an error in the hollow fiber system 100. Namely, the errors may include, but are not limited to, holes in the hollow fiber membrane, holes in conduit, and/or over filled (or under filled) input containers 302. In this example, the timer value 526 indicates to the controller 200 when to terminate execution of the step. In some implementations, the timer value 526 indicates a target time to complete execution of the step. For example, a timer value 526 of a step 510 may include a five minute execution time for the first pump 328 such that after the first pump 328 executes for five minutes the controller 200 successfully completes execution of the step 510 and proceeds to the next step 510.

The air quantity value (e.g., air value) 528 specifies an amount of acceptable air in the fluid (i.e., the input material). That is, the bubble sensors 318 provide the air quantity value to the controller 200 to ensure the filter module 334 receives bubble-free input materials 304, 306, 308, 310. The fluid flow value 530 specifies a maximum or minimum amount of fluid flow. The fluid flow value 530 may represent a volume of fluid per unit of time, for example, 1 liter per minute. In some examples, the bubble sensor 318 provides the fluid flow value 530 to the controller 200. In other examples, the fluid sensor 320 provides the fluid flow value 530 to the controller 200 (e.g., via the fluid flow signal 219).

In the example shown in FIG. 5B, an end trigger threshold 520 may include a fluid sensor 320 identifier, an end trigger type 522, a pressure value 524, and a timer value 526 (each denoted by solid lines) while the air value 528 and the fluid flow value are not included in the end trigger threshold 520 (each denoted by dotted lines). In this example, the end trigger threshold 520 specifies a first fluid sensor 320, a pressure rise end trigger type 522, a maximum pressure value 524, and a timer value 526. Here, each value may include a logical “OR” or “AND” operation such that only one of the values must be satisfied to satisfy the end trigger threshold 520 or all of the values must be satisfied to satisfy the end trigger threshold 520 (e.g., complete or terminate execution of the step 510). Continuing with the example, the watchdog timeout value 526 may indicate a target execution time of two minutes for the pumps 328, 332 to successfully complete the step 510 and the maximum pressure value 524 may indicate if the one of the fluid sensors 320 exceeds 50 PSI to terminate execution of the step 510 and/or the recipe 500. Here, if either condition is met the controller 200 completes execution or terminates execution of the step 510.

In an alternative example, the timer value 526 may indicate the target execution time of two minutes for the pumps 328, 332 and the pressure value indicates a target pressure of 25 PSI to complete execution of the step 510. In this alternative example, the logical operation may require both conditions to be met before the controller 200 completes execution of the step. However, in some instances, the logical operation may only require one of the conditions to be met for the controller 200 to complete execution of the step 510. In some examples, an end trigger threshold 520 specifies a timed end trigger type 522, a timer value 526, at a first fluid sensor 320. In other examples, an end trigger threshold 520 specifies a fluid to air end trigger type 522, a timer value 526, at a third bubble sensor 318. Thus, the end trigger threshold 520 defines operative parameters that define acceptable and/or target operating conditions of the hollow fiber system. As used herein, the operative parameters may refer collectively to at least one of the end trigger type 522, pressure value 524, timer value 526, air value 528, fluid flow value 530, and/or any signals received from the controller 200 receives from the hollow fiber system (e.g., signals 211, 213, 215, 217, 219, 229 as shown in FIG. 1D).

In some scenarios, a user of the hollow fiber system 100 may create and store a recipe 500 at the memory hardware 220 for execution at a later time. The user creates the recipe 500 via the GUI 180 while the system is in the developer mode. The memory hardware 220 is in communication with the data processing hardware 210 such that the data processing hardware 210 can execute one of the stored recipes 500. The user may create the recipe 500 by providing user inputs to the GUI 180 specifying parameters for each step of the recipe 500. Thereafter, the GUI 180 may receive a request to execute the recipe 500 stored at the memory hardware 220. In response to the request, the controller 200 may retrieve the recipe 500 stored at the memory hardware 220 and execute the recipe 500 at the hollow fiber system 100 using the data processing hardware 210.

In some implementations, the GUI 180 receives a request to modify at least one step of the recipe stored at the memory hardware 220. The modification request may specify at least one parameter to change for the step 510 of the recipe 500. For example, the user input to the GUI 180 may specify to change the plurality of pinch valves 316 from an open state to a closed state at a first step 510 of the recipe 500. In another example, the user input to the GUI 180 modifies a timer value 526 from thirty seconds to sixty seconds for a step 510 of the recipe. After receiving the modification request, the controller 200 updates the at least one step of the recipe 500 stored at the memory hardware 220.

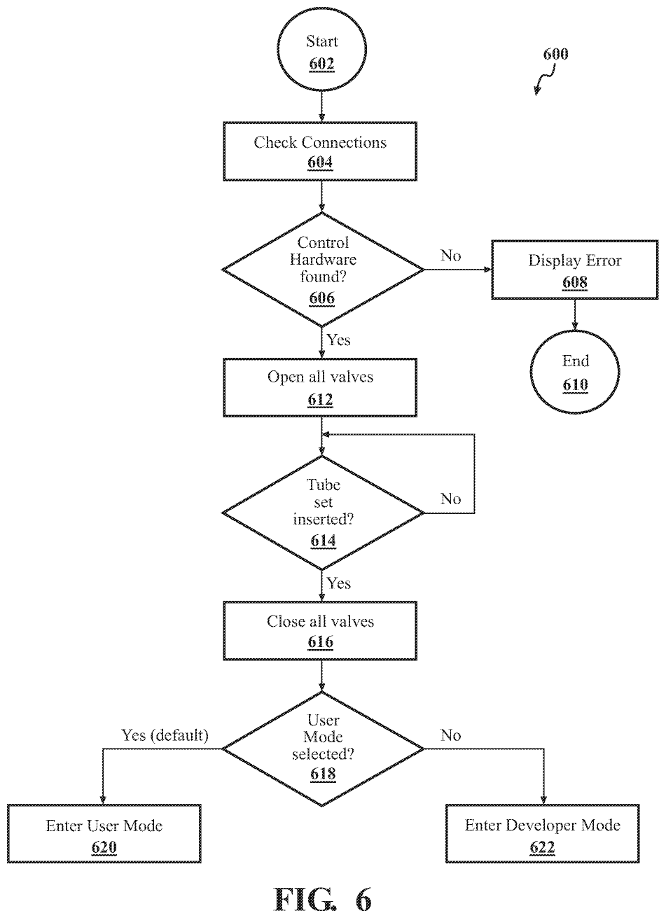

FIG. 6 illustrates an example arrangement of operations for a method 600 of executing an initialization routine via the GUI 180. A user may load an input container 302 or output container 322 into the hollow fiber system 100 to start the method 600 at operation 602. Loading the container 302, 322 means fluidly connecting the container 302, 322 to the hollow fiber system 100. For example, the welder 190 may load the container 302, 322 by welding the container 302, 322 to a conduit of the hollow fiber system 100. At operation 604, the method 600 includes executing the initialization routine at the GUI 180 to check communicative and/or electrical connections for the components of the hollow fiber system 100. The initialization routine may execute in response to a user input at the GUI 180 or in response to a determination that the controller 200 will execute a recipe 500. At operation 606, if control hardware (e.g., controller 200) is not found during the initialization routine, the method 600 proceeds to operation 608 and displays an error to the user via the GUI 180. After displaying the error the method 600 ends at operation 610. Alternatively, if the control hardware is found during the initialization routine, the method 600 proceeds to operation 612 and opens all valves (e.g., pinch valves 316 and flow valves 314). With all valves open, at operation 614, the method 600 includes determining whether a tube set 160 (FIG. 1C) is loaded into the hollow fiber system 100. Alternatively, the method 600 may determine whether an input container 302 or an output container 322 is loaded into the hollow fiber system 100. When no tube set 160 is inserted, the method 600 waits until the tube set 160 is inserted. Alternatively, when the tube set 160 is inserted, at operation 616, the method 600 includes closing all valves. The controller may close all flow valves 314 and/or all pinch valves 316. After closing all the valves, at operation 618, the GUI 180 may receive a user input to operate in user mode at operation 620 or a user input to operate in developer mode at operation 622.

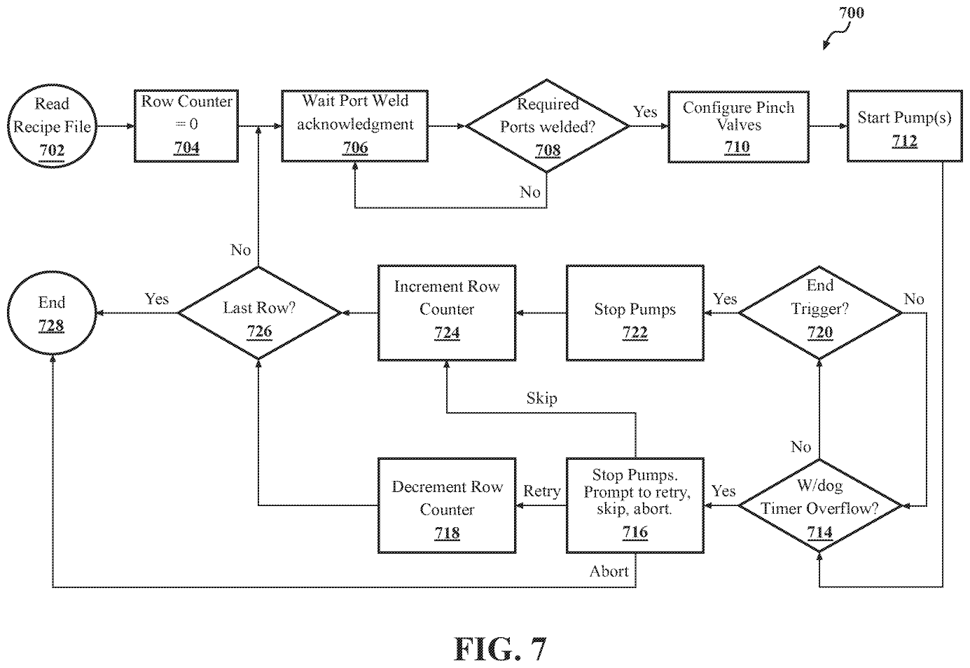

FIG. 7 illustrates an example arrangement of operations for a method 700 for executing each step 510 of a recipe 500. Here, the data processing hardware 210 of the controller 200 may execute each operation of the method 700. At operation 702, the method 700 reads a recipe 500 stored at memory hardware 220 of the controller 200. Here, the recipe 500 may include a data file that includes a row that corresponds to each step 510 of the recipe 500. At operation 704, the method 700 sets a row counter to zero such that the controller 200 begins execution at the first step 510 of the recipe 500. At operation 708, the method 700 determines whether the welder 190 welded the required ports. In some examples, the user provides a user input via the GUI 180 to indicate whether the required ports are welded. Alternatively, the controller 200 may determine whether the required ports are welded. When the required ports are not welded, the method 700 proceeds to operation 706 and waits for a port weld acknowledgment before proceeding to operation 710. If the required ports are welded, at operation 710, the method 700 instructs the plurality of pinch valves 316 to move to an open state or closed state according to the valve configuration state 512 for the current step 510 of the recipe 500. The controller 200 may instruct the plurality of pinch valves 316 via the pinch valve instruction signal 216. Thereafter, at operation 712, the method 700 instructs the pumps 328, 332 to operate according to the pump direction state 514 and/or pump speed 516 of the step 510.

At operation 714, the method 700 determines whether an execution time of the currently executing step 510 of the recipe 500 exceeds a timer value 526 (e.g., watchdog timer value) of the step. If execution time of the currently executing step 510 exceeds the timer value 526, at operation 716, the method 700 stops the pumps 328, 332 (e.g., sets pump speed 516 to zero via the pump instruction signal 231) and prompts the user to retry, skip, or abort the step 510 via the GUI 180. When the user provides a user input to abort the currently executing step 510, the method 700, at operation 728, terminates execution of the entire recipe 500. Alternatively, when the user provides a user input to retry the currently executing step 510, the method 700, at operation 718, decrements the row counter and advances to operation 726. In particular, decrementing the row counter allows the controller 200 to return to a previous step 510 (or return to the beginning of the currently executing step 510) of the recipe 500 and retry the step 510 that incurred a watchdog failure. When the user provides a user input to skip the currently executing step 510, the method 700, at operation 724, increments the row counter and skips the currently executing step 510 even though the currently executing step 510 has not successfully completed execution.

In the example shown, if execution time of the step 510 does not exceed the timer value 526 the method 700, at operation 720 determines whether the end trigger threshold 520 of the step 510 is satisfied. Here, if the end trigger threshold 520 is not satisfied, the method 700 returns to operation 714. When the end trigger threshold 520 is satisfied, at operation 722, the method 700 initiates a pump rate adjustment procedure.

Here, the pump rate adjustment procedure includes stopping operation of the pumps 328, 332 for a predetermined period of time. Alternatively, the pump rate adjustment procedure includes reversing pump direction 514 of the pumps 328, 332. Thereafter, at operation 724, the method 700 increments the row counter (e.g., moves to the next step 510 of the recipe 500).

At operation 726, the method 700 determines whether the currently executing step 510 of the recipe 500 is a last step of the recipe 500. When the currently executing step 510 of the recipe 500 is the last step 510 of the recipe 500, at operation 728, the method 700 completes execution of the recipe 500. When the currently executing step 510 of the recipe 500 is not the last step 510 of the recipe 500, the method 700 returns to operation 706. The operations 702-726 are repeated for each step 510 of the recipe 500 until the last step 510 of the recipe 500 completes.

In some implementations, the controller 200 executes a recipe 500 that includes pressure based control. Pressure based control includes performing operations in response to the controller 200 determining that the pressure value 524 exceeds a maximum threshold or does not exceed a minimum threshold. For example, the controller 200 may instruct the first pump 328 to operate at a first pump rate 516 (via the pump instruction signal 231) to provide a first flow of at least one of the input materials 304, 306, 308, 310 to the first intra-capillary port 360a according to a first step 510 of the recipe 500. Alternatively, the controller 200 may instruct the second pump 332 to operate at the first pump rate 516 to provide the first flow of at least one of the input materials 304, 306, 310 to the first extra-capillary port 364a. For example, the first pump 328 may provide the cells 306 and/or the vector 310 to the first intra-capillary port 360a. In another example, the first pump 328 may provide the culture media 308 and/or the transduction media 304 to the first intra-capillary port 360a. Here, at the first step 510 of the recipe 500, the controller 200 obtains a first fluid flow parameter 530 of the fluid flow of the at least one input material 304, 306, 308, 310 from the fluid sensor 320a. In this example, the first step 510 of the recipe 500 may include an end trigger threshold 520 based on a maximum pressure value 524 to prevent damaging components of the hollow fiber system 100 (e.g., bursting the conduit). Thus, when the pressure value 524 of first flow exceeds a maximum pressure threshold, the controller 200 may initiate a pump rate adjustment procedure.

FIG. 8 illustrates an example arrangement of operations for a method 800 of executing a pump rate adjustment procedure. At operation 802, the method 800 determines whether the end trigger threshold 520 of the respective step 510 is satisfied. In some examples, the controller 200 determines whether the hollow fiber system 100 is within a threshold value (e.g., ten percent) of the end trigger threshold 520. If the end trigger threshold 520 is not satisfied, at operation 804, the controller 200 obtains a first measured fluid flow parameter 530_{M1} from a fluid sensor 320. The controller 200 obtains the first measure fluid flow parameter 530_{M1} via a first fluid flow signal 219. Moreover, the controller 200 compares the measured first measured fluid flow parameter 530_{M1} to a first fluid flow parameter threshold 530_{T1}. For example, the first fluid flow parameter threshold 530_{T1} may be a predetermined maximum pressure value associated with the components of the system, such as 28 pounds per square inch (PSI). If the first measured fluid flow parameter 530_{M1} does not exceed the first fluid flow parameter threshold 530_{T1}, the method 800 returns to operation 802. If the first measured fluid flow parameter 530_{M} does exceed the first fluid flow parameter threshold 530_{T1}, at operation 806, the method 800 initiates the pump rate adjustment procedure by operating the pump 328, 332 in a reverse-flow state (e.g., reversing the pump direction state 514). Operating the pump 328, 332 in the reverse-flow state reverses the fluid flow direction and is configured to reduce the first fluid flow parameter 530. Additionally or alternatively, the pump rate adjustment procedure may include stopping operation of the pump 328, 332 for a predetermined period (e.g., period of time). In some examples, the predetermined period is a pressure adjustment period.

At operation 808, the method 800 obtains a second measured fluid flow parameter 530_{M2} of the first flow from the fluid sensor 320. The controller 200 obtains the second measured fluid flow parameter 530_{M2} via a second fluid flow signal 219. Here, the controller 200 obtains the second measured fluid flow parameter 530_{M2} after a predetermined amount of time from reversing the pumps 328, 332 at operation 806. The controller 200 compares the second measured fluid flow parameter 530_{M2} against a second fluid flow parameter threshold 530_{T2}. The second fluid flow parameter threshold 530_{T2} may be 15 PSI. Notably, the second fluid flow parameter threshold 530_{T2} is less than the first fluid flow parameter threshold 530_{T1}. If the second measured fluid flow parameter 530_{M2} does exceed the second fluid flow parameter threshold 530_{T2}, the method 800 continues to obtain updated value and compare the second measured fluid flow parameter 530_{M2} against the second fluid flow parameter threshold 530_{T2} until the threshold is satisfied. If the second measured fluid flow parameter 530_{M2} does not exceed the second fluid flow parameter threshold 530_{T2}, at operation 810, the method 800 initiates the pump rate adjustment procedure depending on the comparison. In particular, the pump rate adjustment procedure reduces the speed of the pump 328, 332 by fifty percent (e.g., operates the pump 328, 332 at a second pump rate 516 depending on the comparison).

At operation 812, the method 800 obtains a pump speed (i.e., measured pump speed) 516_{M} of the pump 328, 332 after reducing the pump speed 516 at operation 810. Moreover, the controller 200 compares the measured pump speed 516_{M} against a pump speed threshold 516_{T}. Here, the controller 200 determines whether the measured pump speed 516_{M} satisfies the pump speed threshold 516_{T}. If the measured pump speed 516_{M} exceeds the pump speed threshold 516_{T}, at operation 814, the method 800 restarts the pump 328, 332. Alternatively, if the measured pump speed 516_{M} does not exceed the pump speed threshold 516_{T}, at operation 816, the method 800 prompts the user to skip or abort the currently executing step 510 of the recipe 500 via the GUI 180. When the user selects to skip the currently executing step 510 of the recipe 500, at operation 820, the method 800 proceeds to the next step 510 of the recipe 500. When the user selects to abort the currently executing step 510 of the recipe 500, at operation 818, the method 800 instructs the pump 328 into an idle state.

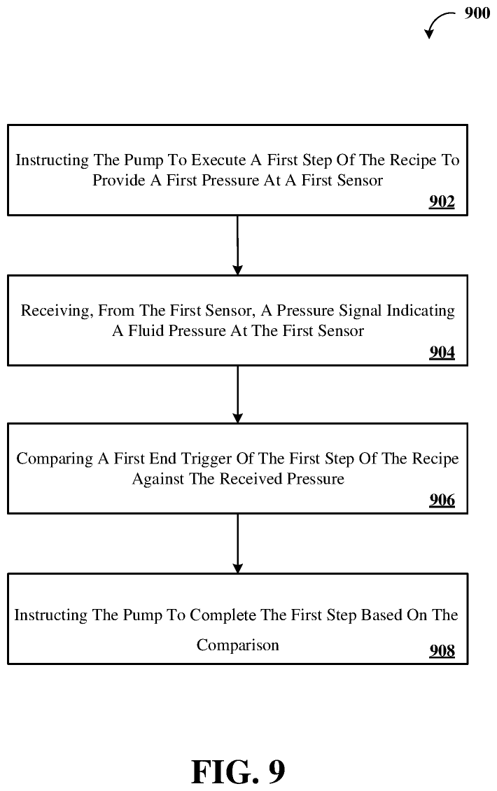

FIG. 9 illustrates an example arrangement of operations for a method 900 of executing an example recipe 500 at the hollow fiber system 100. Here, the controller 200 may execute each operation of the method 900. At operation 902, the method 900 includes the controller 200 instructing the one or more pumps 328, 332 to execute a first step of the recipe to provide a first pressure at a first fluid sensor 320. Here, the first pressure at the first fluid sensor 320 may be specified by the first step 510 of the recipe 500. Moreover, the controller 200 may send a pump instruction signal 231 to the one or more pumps 328, 332 to operate at a first pump speed 516 specified by the recipe 500. In this example, the controller 200 may also send the pump instruction signal 231 that further indicates a pump operating direction (e.g., according to the pump direction 514 of the first step 510 of the recipe 500) to the one or more pumps 328, 332 to operate in a first direction. Further, the controller 200 may instruct the pinch valves 316 to open or close according to the valve configuration 512 of the first step 510 of the recipe 500.

At operation 904, the method 900 includes receiving a fluid flow signal 219 (e.g., pressure signal) indicating a measured operating parameter at the first fluid sensor. Here, the operating parameter may include a pressure value 524 and/or a fluid flow value 530 at the first fluid sensor 320. At operation 906, the method 900 includes the controller 200 comparing a first end trigger threshold 520 of the first step 510 of the recipe 500 against the received fluid flow signal 219 (e.g., pressure signal) from the first fluid sensor 320. That is, the first step 510 of the recipe may specify a maximum pressure value 524 of the fluid pressure at the first fluid sensor 320. The maximum pressure value 524 may represent a target pressure such that, when the pressure reaches the maximum pressure value, the controller 200 has successfully executed the first step 510 and the controller 200 can complete the first step 510 of the recipe 500 and begin execution of the second step 510 of the recipe 500. Alternatively, the maximum pressure value 524 may represent a failsafe pressure. Here, when the pressure reaches the maximum pressure value 524, the controller 200 terminates the first step 510 of the recipe 500. That is, the pressure value 524 may reach a pressure that can damage the hollow fiber system 100, and thus, the controller 200 terminates execution of the first step 510 to protect the hollow fiber system 100. Alternatively, the controller 200 may bypass the first step 510 or prompt the user via the GUI 180. Thus, at operation 908, the method 900 includes the controller 200 instructing the one of the pumps 328, 332 to complete the first step 510 of the recipe 500 based on the comparison of the received pressure signal and the first end trigger threshold 520. In some instances, when the controller 200 terminates the first step 510 of the recipe, the controller 200 further skips or retries the first step 510 of the recipe.

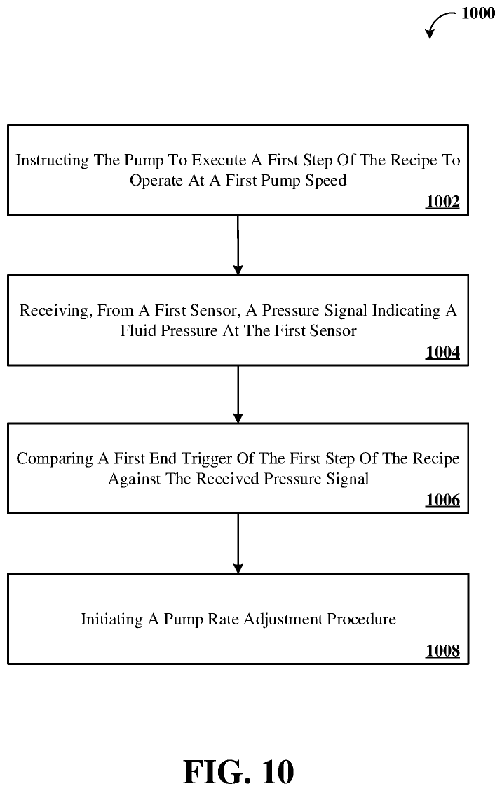

FIG. 10 illustrates an example arrangement of operations for a method 1000 of executing an example recipe 500 at the hollow fiber system 100. Here, the controller 200 may execute each operation of the method 1000. At operation 1002, the method 1000 includes the controller 200 instructing the one or more pumps 328, 332 to execute a first step of the recipe 500 to operate at a first pump speed 516 according to the first step 510 of the recipe 500. The controller 200 may determine the first pump speed 516 using a first target pressure specified in the recipe 500. In this example, the controller 200 may also send a pump instruction signal 231 to indicate a pump operating direction (e.g., according to the pump direction 514 of the first step 510 of the recipe 500) to the one or more pumps 328, 332 to operate in a first direction. Further, the controller 200 may instruct the pinch valves 316 to open or close according to the valve configuration 512 of the first step 510 of the recipe 500 to define a fluid flow path for the one or more pumps 328, 332.

At operation 1004, the method 1000 includes receiving a fluid flow signal 219 (e.g., pressure signal) that indicates a pressure value 524 measured at a first fluid sensor 320 at the controller 200. Here, the pressure value 524 is an operative parameter. At operation 1006, the method 1000 includes the controller 200 comparing a first end trigger threshold 520 of the first step 510 of the recipe 500 against the received pressure signal from the first fluid sensor 320. Here, first step 510 of the recipe specifies a maximum pressure value 524 of the fluid pressure at the first fluid sensor 320. The maximum pressure may represent a burst pressure value of the filter module 334 or the conduit. As such, at operation 1008, the method 1000 includes the controller 200 initiating a pump rate adjustment procedure based on the comparison that the received pressure signal exceeds the first end trigger threshold 520.

The pump rate adjustment procedure may include the controller 200 operating the one or more pumps 328, 332 in a reverse-flow state (e.g., changing the pump direction 514) for a predetermined amount of time. In some examples, the pump direction 514 alternatives between the first direction and the second direction at each step 510 of the recipe 500. Alternatively, the pump rate adjustment procedure may include the controller 200 stopping operation of the one or more pumps 328, 332 (e.g., operating at zero pump speed 516) for a predetermined amount of time.

Notably, both reversing direction and stopping operation of the one or more pumps 328, 332 reduces the pressure at the first fluid sensor 320 such that the measured pressure at the first pressure sensor does not satisfy the first end trigger threshold 520 (i.e., the measured pressure is within an acceptable range). Accordingly, during execution of the first step 510 of the recipe 500 the received pressure signal can exceed the first end trigger threshold 520 and the controller 200 can initiate the pump rate adjustment procedure thereby allowing the controller 200 to continue execution of the first step 510 rather than terminating the first step 510. Moreover, the controller 200 initiates the pump adjustment rate procedure responsive to the comparison of the received pressure signal and the first end trigger threshold 520 (e.g., without any user input) reducing user interaction with the hollow fiber system 100 to perform cell therapy transduction.

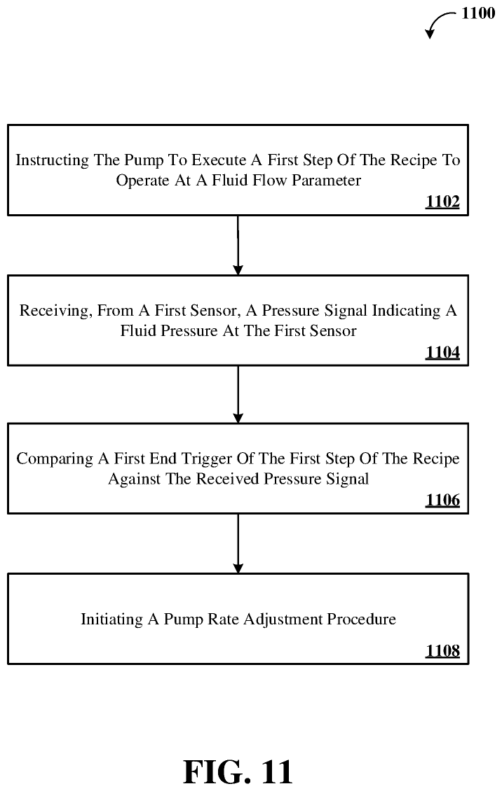

FIG. 11 illustrates an example arrangement of operations of for a method 1100 of executing an example recipe 500 at the hollow fiber system 100. Here, the controller 200 may execute each operation of the method 1000. At operation 1102, the method 1000 includes the controller 200 instructing the one or more pumps 328, 332 to execute a first step of the recipe 500 to operate at a first fluid flow parameter 530 (e.g., a constant fluid flow parameter) at a fluid sensor 320 (or bubble sensor 318). In this example, the controller 200 may also send a pump instruction signal 331 that indicates a pump operating direction (e.g., according to the pump direction 514 of the first step 510 of the recipe 500) to the one or more pumps 328, 332 to operate in a first direction. Further, the controller 200 may instruct the pinch valves 316 to open or close according to the valve configuration 512 of the first step 510 of the recipe 500 to define a fluid flow path for the one or more pumps 328, 332.

At operation 1104, the method 1100 includes receiving a fluid flow value 219 (e.g., pressure signal) that indicates a fluid pressure (e.g., pressure value 524) at the first fluid sensor 320. Here, the pressure value is a measured operative parameter. At operation 1106, the method 1100 includes the controller 200 comparing a first end trigger threshold 520 of the first step 510 of the recipe 500 against the received pressure signal from the first fluid sensor 320. That is, the first step 510 of the recipe may specify a maximum pressure value 524 of the fluid pressure at the first fluid sensor 320. Here, the maximum pressure value 524 represents a failsafe pressure causing the controller 200 to reduce the fluid pressure at the first fluid sensor 320. At operation 1108, the method 1100 includes the controller 200 initiating a pump rate adjustment procedure based upon the comparison of the received pressure signal and the first end trigger threshold 520. That is, the controller 200 may instruct the pump 328, 332 to operate at a second pump speed (e.g., lower than the first pump speed), stop operation of the pumps 328, 332, and/or reduce the fluid flow direction of the pumps 328, 332. Notably, the controller 200 may operate the pumps 328, 332 at constant fluid flow parameter 530 (without exceeding the maximum pressure) such that the controller 200 completes the recipe 500 in the quickest amount of time. In particular, the controller 200 may determine a maximum fluid flow parameter 530 to operate the hollow fiber system 100 at without exceeding the maximum pressure. Thus, the controller 200 operates the pumps 328, 332 at the maximum speed to minimize transduction time at the hollow fiber system 100.

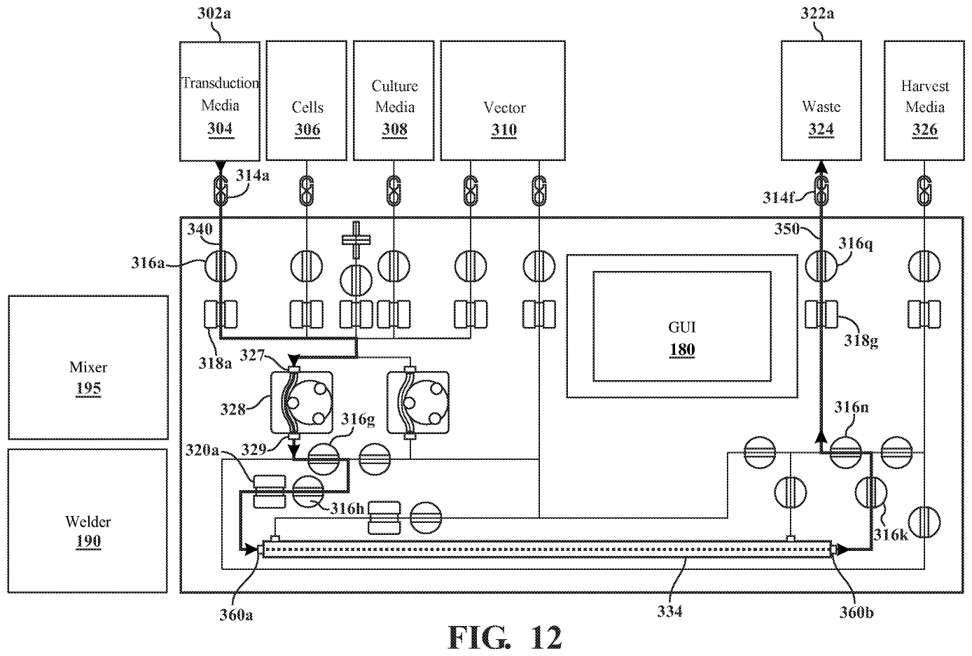

FIGS. 12-24 illustrate example schematic views of fluid flow paths through the hollow fiber system 100 according to a plurality of steps 510 of a recipe 500. In particular, each figure in FIGS. 12-24 illustrates one step 510 from the plurality of steps 510 of the 500. It is understood that the plurality of steps 510 as shown in FIGS. 12-24 illustrate an example recipe 500 only and are not meant to limit this disclosure in any way. For example, a different example recipe 500 may repeat any of the plurality of steps 510 any number of times, include more or fewer steps 510, and/or perform the plurality of steps 510 in a different order than the example recipe 500 illustrated in FIGS. 12-24. For example, a recipe 500 may only include a single step 510 from the example recipe 500 illustrated in FIGS. 12-24. In other examples, a recipe 500 may include one or more additional steps 510 that are not illustrated in FIGS. 12-24 in addition to, or in lieu of, any of the plurality of steps 510 of the example recipe 500. The one or more additional steps 510 may include any configuration (e.g., valve direction 512, pump direction 514, pump speed 516, step type 518, end trigger threshold 520, etc.) of the hollow fiber system 100 as described above in this disclosure. In yet other examples, any step 510 of the recipe 500 may repeat execution any number of times before proceeding to a next step 510 of the recipe 500.

It is understood that FIGS. 12-24 include the same components as FIG. 3, however, for the sake of clarity, only a portion of the components are labeled in FIGS. 12-24. For example, a pump that is actively pumping fluid is labeled while a pump that is not actively pumping fluid is not, open valves are labeled while closed valves are not, and sensors with fluid actively passing through the sensor are labeled while sensors without fluid actively passing through the sensor are not labeled. In these examples, the direction of arrows indicates fluid flow in the hollow fiber system 100. Moreover, bold lines represent which conduits have active fluid flow paths and non-bold lines represent conduits without active fluid flow paths. Notably, non-bold lines may still have fluid within, but the fluid is not actively flowing.

FIG. 12 illustrates execution of a first step 510 of an example recipe 500 for priming the intra-capillary space 338 of the filter module 334 with transduction media 304. In the example shown, the controller 200 instructs the hollow fiber system 100 to perform operations according to the example recipe 500. In the particular, the controller 200 instructs the plurality of pinch valves 316 to execute the first step 510 of the example recipe 500 such that pinch valves 316a, 316g, 316h, 316k, 316n, 316q are open and all other pinch valves 316 are closed. Here, a user or the controller 200 may open the flow valves 314a, 314f. Thereafter, the controller 200 instructs the first pump 328 to pump the transduction media 304 from the transduction container 302a to the waste 324 of the waste container 322a via the intra-capillary space 338 of the filter module 334 (shown in FIG. 3B). In this example, the bubble sensors 318a, 318g provide air quantity values to the controller 200 that represents a quantity of air in the transduction conduit 340 and the waste conduit 350 respectively. Moreover, the first fluid sensor 320a provides a pressure value and/or a fluid flow value to the controller 200 representing the pressure and/or fluid flow at the first intra-capillary port 360a.

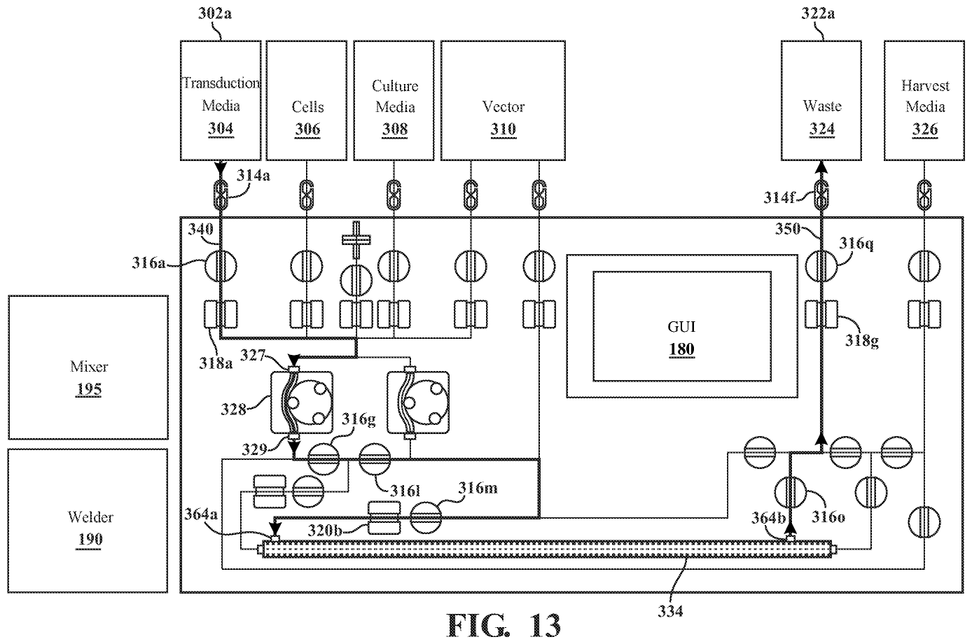

FIG. 13 illustrates execution of a second step 510 of the example recipe 500 for priming the extra-capillary space 339 of the filter module 334 (shown in FIG. 3B) with transduction media 304. In the example shown, the controller 200 instructs the hollow fiber system 100 to perform operations according to the example recipe 500. In particular, the controller 200 instructs the plurality of pinch valves 316 to execute the second step 510 of the example recipe 500 such that pinch valves 316a, 316g, 316l, 316m, 316o, 316q are open and all other pinch valves 316 are closed. Here, the user or the controller 200 may open the flow valves 314a, 314f. Thereafter, the controller 200 instructs the first pump 328 to pump the transduction media 304 from the transduction container 302a to the waste 324 of the waste container 322a via the extra-capillary space 339 of the filter module 334. In this example, the bubble sensors 318a, 318g provide air quantity values to the controller 200 that represents a quantity of air in the transduction conduit 340 and the waste conduit 350 respectively. Moreover, the second fluid sensor 320b provides a pressure value and/or a fluid flow value to the controller 200 representing the pressure and/or fluid flow at the first extra-capillary port 364a.

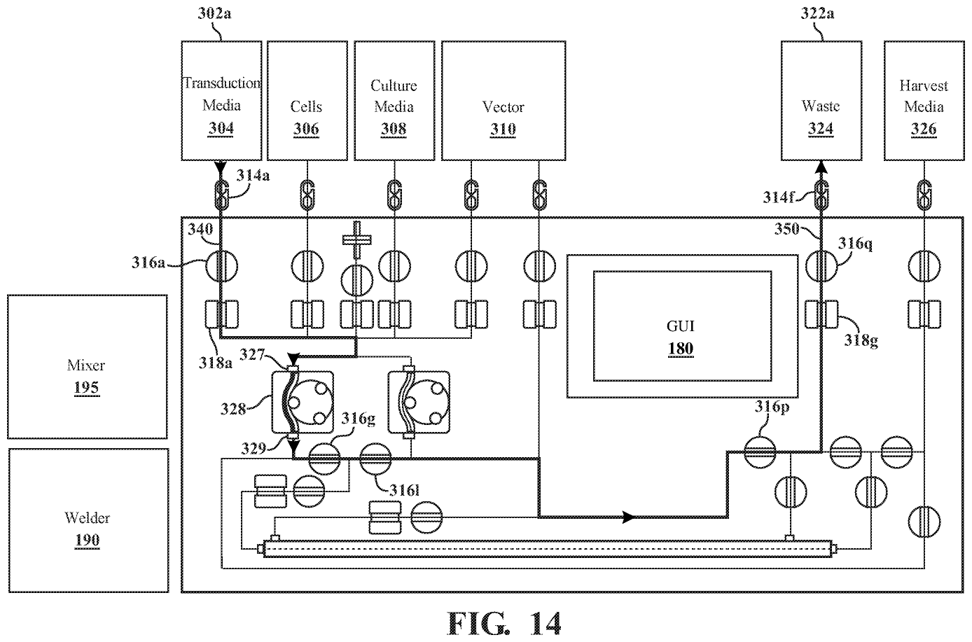

FIG. 14 illustrates execution of a third step 510 of the example recipe 500 for priming the extra-capillary bypass with the transduction media 304. In the example shown, the controller 200 instructs the hollow fiber system 100 to perform operations according to the example recipe 500. In particular, the controller 200 instructs the plurality of pinch valves 316 to execute the third step 510 of the recipe 500 such that pinch valves 316a, 316g, 316l, 316p, 316q are open and all other pinch valves 316 are closed. Here, the user or the controller 200 may open the flow valves 314a, 314f. Thereafter, the controller 200 instructs the first pump 328 to pump the transduction media 304 from the transduction container 302a to the waste 324 of the waste container 322a via extra-capillary bypass conduit. In this example, the bubble sensors 318a, 318g provide air quantity values to the controller 200 that represents a quantity of air in the transduction conduit 340 and the waste conduit 350 respectively.

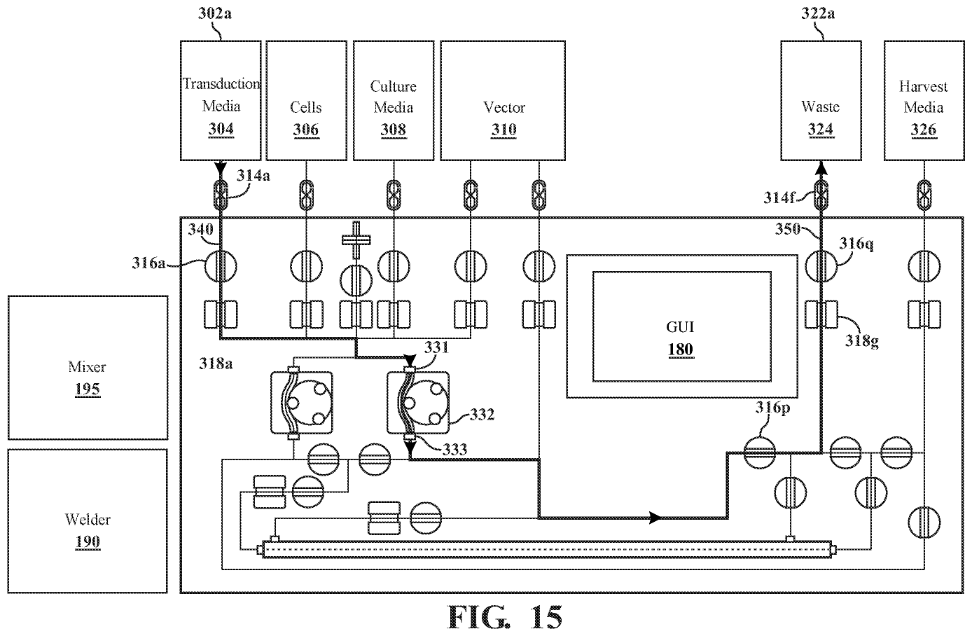

FIG. 15 illustrates execution of a fourth step 510 of the example recipe 500 for priming the second pump 332 and the extra-capillary bypass with the transduction media 304. In the example shown, the controller 200 instructs the hollow fiber system 100 to perform operations according to the example recipe 500. In particular, the controller 200 instructs the plurality of pinch valves 316 to execute the fourth step 510 of the example recipe 500 such that pinch valves 316a, 316p, 316q are open and all other pinch valves 316 are closed. Here, the user or the controller 200 may open the flow valves 314a, 314f. Thereafter, the controller 200 instructs the second pump 332 to pump the transduction media 304 from the transduction container 302a to the waste 324 of the waste container 322a via extra-capillary bypass conduit. In this example, the bubble sensors 318a, 318g provide air quantity values to the controller 200 that represents a quantity of air in the transduction conduit 340 and the waste conduit 350 respectively.

FIG. 16 illustrates execution of a fifth step 510 of the example recipe 500 for integrity testing. In the example shown, the controller 200 instructs the hollow fiber system 100 to perform operations according to the example recipe 500. In particular, the controller 200 instructs the plurality of pinch valves 316 to execute the fifth step 510 of the example recipe 500 such that pinch valves 316c, 316g, 316h. Here, the user or the controller 200 may open the air filter 312 to allow air to be pump into the air filter conduit 342 (shown in FIG. 1C). Thereafter, the controller 200 instructs the first pump 328 to pump the air through the air filter 312 to purge transduction media 304 from the filter module 334. That is, the controller 200 may open pinch valves 316k, 316n, 316q and flow valve 314f to pump air through the filter module 334 and purge the transduction media 304 into the waste container 322a (this configuration is not shown in FIG. 16). Thereafter, the controller 200 may close pinch valves 316k, 316n, 316q and pressurize the intra-capillary space 338 (shown in FIG. 3B) to a first pressure target (e.g., 15 PSI). Here, the first fluid sensor 320a may indicate the pressure value. After reaching the pressure target, the controller 200 may stop operation of the first pump 328 and continue to obtain and monitor the pressure at the first fluid sensor 320a. Moreover, the controller 200 obtains and monitors air quantity value at the bubble sensor 318 to determine whether any bubbles are present. When the pressure drop exceeds a threshold value and/or the air quantity value exceeds threshold, the integrity test fails.

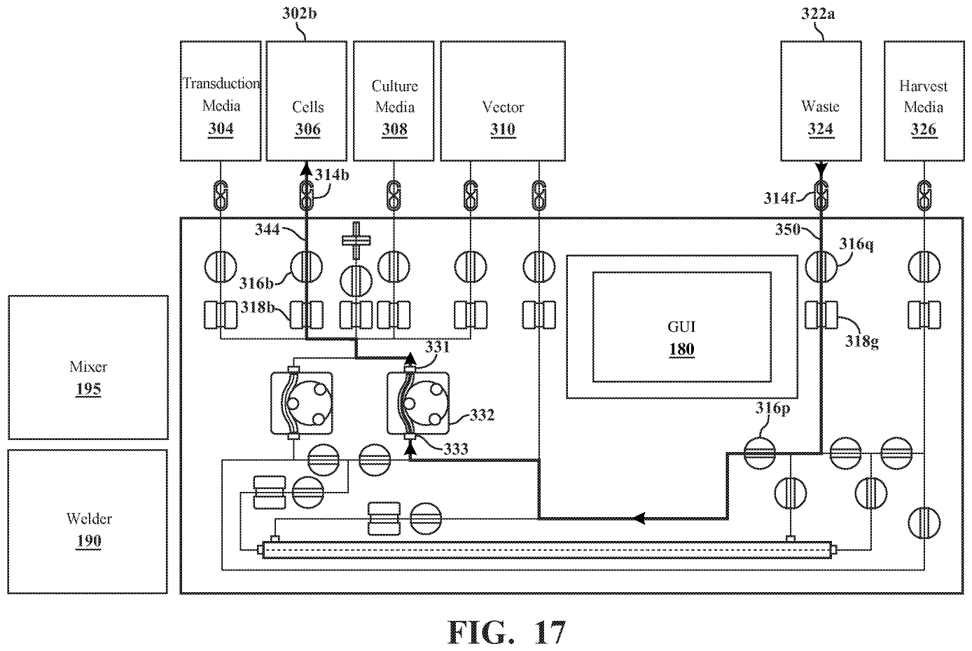

FIG. 17 illustrates execution of a sixth step 510 of the example recipe 500 for priming the cell conduit 344 with fluid after loading the cell container 302b. Thus, the welder 190 may weld the cell container 302b to the cell conduit 344 to fluidly connect the cell container 302b to the hollow fiber system 100. In the example shown, the controller 200 instructs the hollow fiber system 100 to perform operations according to the example recipe 500. In particular, the controller 200 instructs the plurality of pinch valves 316 to execute the sixth step 510 of the recipe 500 such that pinch valves 316b, 316p, 316q are open and all other pinch valves 316 are closed. Here, a user or the controller 200 may open the flow valves 314b, 314f. Thereafter, the controller 200 instructs the second pump 332 to pump the waste 324 from the waste container 322a to the cells 306 of the cell container 302b via the extra-capillary bypass conduit. Notably, the second pump 332 pumps the fluid in a second direction from the output container 322 to the input container 302. Executing the sixth step 510 purges air from the cell conduit 344 before loading the cells 306 into the hollow fiber system 100. Advantageously, this ensures that no air bubbles are pushed into the filter module 334 along with the cells 306 that may interfere with transduction. In this example, the bubble sensors 318b, 318g provide air quantity values to the controller 200 that represents a quantity of air in the cell conduit 344 and the waste conduit 350 respectively. The bubble sensors 318b, 318g reporting the absence of air in the cell conduit 344 signifies the completion of the sixth step 510.

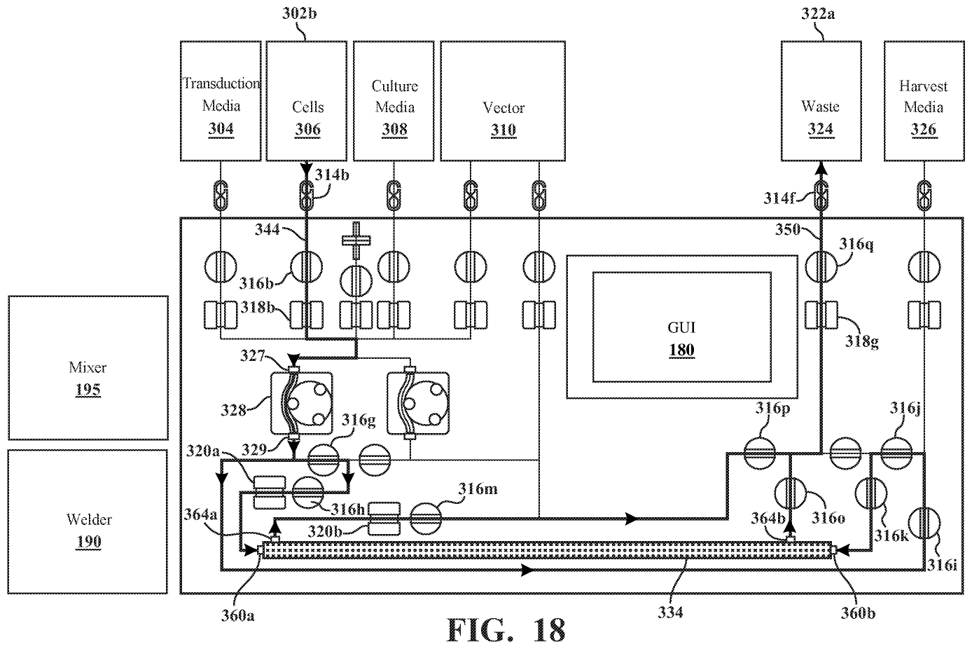

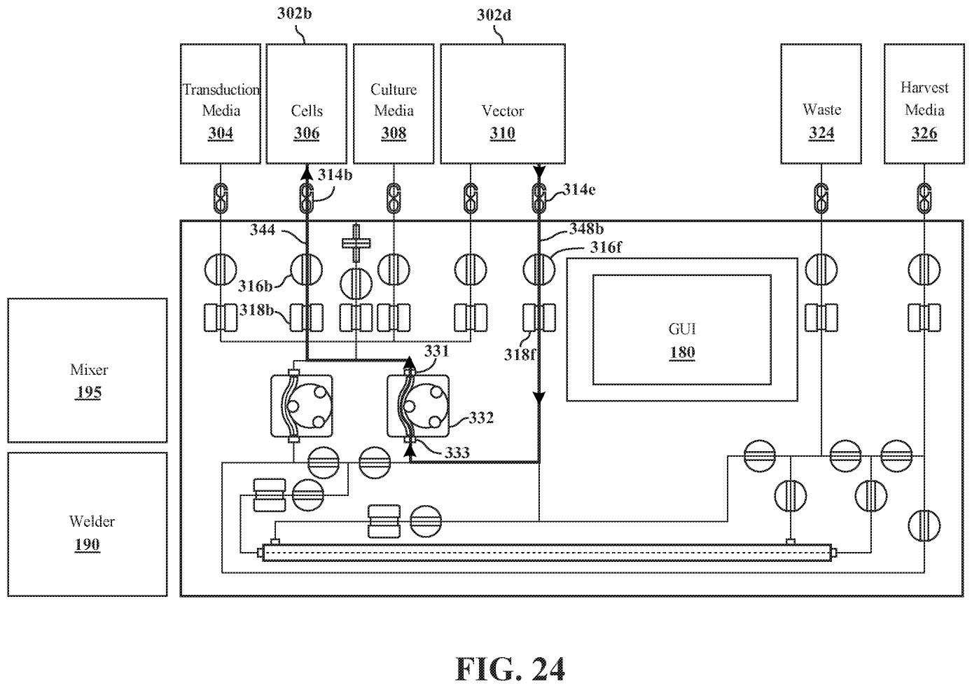

FIG. 18 illustrates execution of a seventh step 510 of the example recipe 500 for loading cells 306 into the filter module 334. Loading into the filter module 334 refers to pumping an input material 304, 306, 308, 310 into the intra-capillary space 338 and/or extra-capillary space 339 of the filter module 334. In some implementations, the mixer 195 mixes the cells 306 with the vector 310 and loads a mixture thereof, as described in reference to FIG. 24, into the filter module 334. In the example shown, the controller 200 instructs the hollow fiber system 100 to perform operations according to the example recipe 500. In the particular, the controller 200 instructs the plurality of pinch valves 316 to execute the seventh step 510 of the example recipe such that pinch valves 316b, 316g, 316h, 316i, 316j, 316k, 316m, 316o, 316p, 316q are open and all other pinch valves 316 are closed. Here, a user or the controller 200 may open the flow valves 314b, 314f. Thereafter, the controller 200 instructs the first pump 328 to pump the cells 306 (or the mixture of cells 306 and the vector 310 as described in FIG. 24) from the cell container 302b to the waste 324 of the waste container 322a via the intra-capillary space 338 and the extra-capillary space 339 of the filter module 334.

More specifically, the first pump 328 pumps the cells 306 (or the mixture of cells 306 and the vector 310 as described in FIG. 24) into both the first intra-capillary port 360a and the second intra-capillary port 360b to create a counter-flow of cells within the intra-capillary space 338. As the cells 306 flow from opposite ends of the intra-capillary space 338 of the filter module 334, the counter-flows of the cells 306 collide and/or coalesce at a common region within the intra-capillary space 338 causing the cells 306 to be captured within the filter module 334. Moreover, the fluid that the cells 306 are suspended in may flow from the intra-capillary space 338 to the extra-capillary space 339 thereby exiting the extra-capillary space 339 through the first extra-capillary port 364a and the second extra-capillary port 364b through the waste conduit 350 to the waste container 322a. Notably, the cells 306 (or the mixture of cells 306 and the vector 310 as described in FIG. 24) do not flow from the intra-capillary space 338 to the extra-capillary space 339, but rather only the fluid that the cells 306 are suspended in.

In this example, the bubble sensors 318a, 318g provide air quantity values to the controller 200 that represents a quantity of air in the transduction conduit 340 and the waste conduit 350 respectively. Moreover, the first fluid sensor 320a provides a pressure value and/or a fluid flow value to the controller 200 representing the pressure and/or fluid flow of cells 306 into the first intra-capillary port 360a. The second fluid sensor 320b provides a pressure value and/or a fluid value to the controller 200 representing the pressure and/or fluid flow of cells 306 flowing from the first extra-capillary port 364a.

FIG. 24 illustrates execution of an optional mixing step 510 of the example recipe 500 for transporting the vector 310 from the vector container 302d to the cell container 302b, which may be executed prior to loading the filter module 334, as discussed with respect to FIG. 18. In the example shown, the controller 200 instructs the hollow fiber system 100 to perform operations according to the example recipe 500. In particular, the controller 200 instructs the plurality of pinch valves 316 to execute the mixing step 510 of the example recipe 500 such that pinch valves 316b, 316f are open and all other pinch valves 316 are closed. Here, a user or the controller may open the flow valves 314b, 314e. Thereafter, the controller 200 instructs the second pump 332 to pump the population of vector particles (e.g., the vector 310) from the vector container 302d to the cell container 302b that includes the population of cells 306. Notably, pumping the vector particles to the cell container 302b causes the vector 310 to combine with the cells 306 to generate a mixture thereof.

In some examples, the controller 200 instructs the mixer 195 to mix the cells 306 and the vector 310. That is, the mixer 195 is in fluid communication with the cell container 302b and is configured to mix the cells 306 and the vector 310. Thereafter, the controller 200 may instruct mixer 195 to return the mixture to the cell container 302b. Alternatively, a user may manually encourage mixing of the cells 306 and the vector 310 within the cell container 302b by manipulating or massaging the cell container 302b.

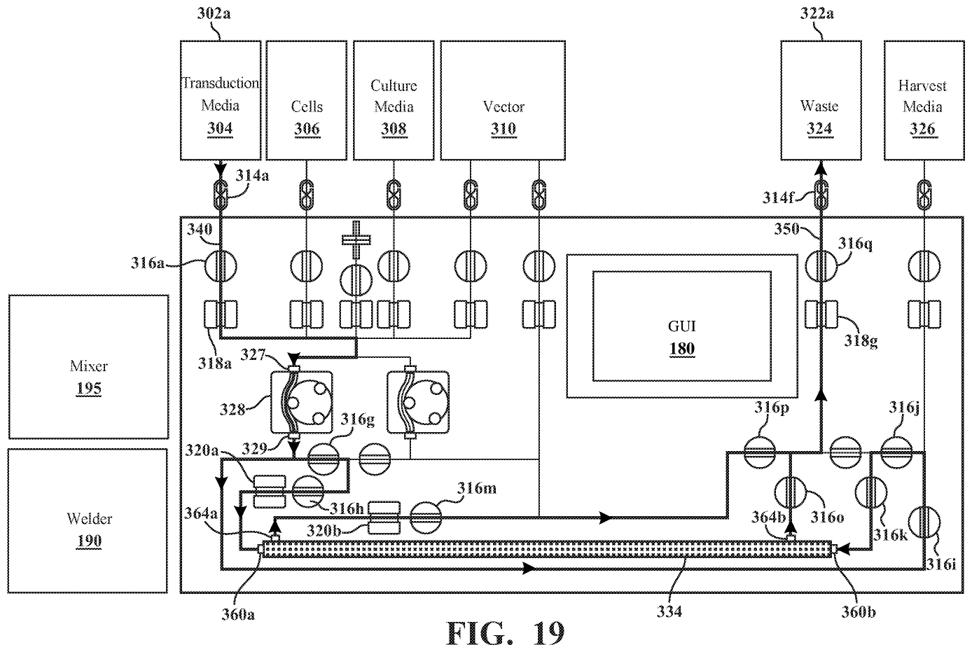

FIG. 19 illustrates execution of an eighth step 510 of the example recipe 500 for transduction media 304 chase to clear cells 306 from the conduit. That is, the recipe of FIG. 19 may be executed after the execution of the sixth step 510 of the example recipe 500 (FIG. 18) to clear any remaining cells 306 from conduit into the filter module 334. Clearing the remaining cells prevents build-up or settlement of cells in the conduit. In the example shown, the controller 200 instructs the hollow fiber system 100 to perform operations according to the example recipe 500. In the particular, the controller 200 instructs the plurality of pinch valves 316 to execute the eighth step 510 of the example recipe 500 such that pinch valves 316a, 316g, 316h, 316i, 316j, 316k, 316m, 316o, 316p, 316q are open and all other pinch valves 316 are closed. Here, a user or the controller 200 may open the flow valves 314a, 314f. Thereafter, the controller 200 instructs the first pump 328 to pump the transduction media 304 from the transduction container 302a to the waste 324 of the waste container 322a via the intra-capillary space 338 and the extra-capillary space 339 of the filter module 334. The flow of the transduction media 304 pumps any remaining cells 306 into the filter module 334.

More specifically, the first pump 328 pumps the transduction media 304 into both the first intra-capillary port 360a and the second intra-capillary port 360b into the intra-capillary space 338 (as shown in FIG. 3B). The transduction media 304 flows from the intra-capillary space 338 to the extra-capillary space 339 (as shown in FIG. 3B) via the porous membrane and exits the filter module 334 through the first extra-capillary port 364a and the second extra-capillary port 364b through the waste conduit 350 to the waste container 322a. In this example, the bubble sensors 318a, 318g provide air quantity values to the controller 200 that represents a quantity of air in the transduction conduit 340 and the waste conduit 350 respectively. Moreover, the first fluid sensor 320a provides a pressure value and/or a fluid flow value to the controller 200 representing the pressure and/or fluid flow of transduction media 304 into the first intra-capillary port 360a. The second fluid sensor 320b provides a pressure value and/or a fluid value to the controller 200 representing the pressure and/or fluid flow of transduction media 304 flowing from the first extra-capillary port 364a.

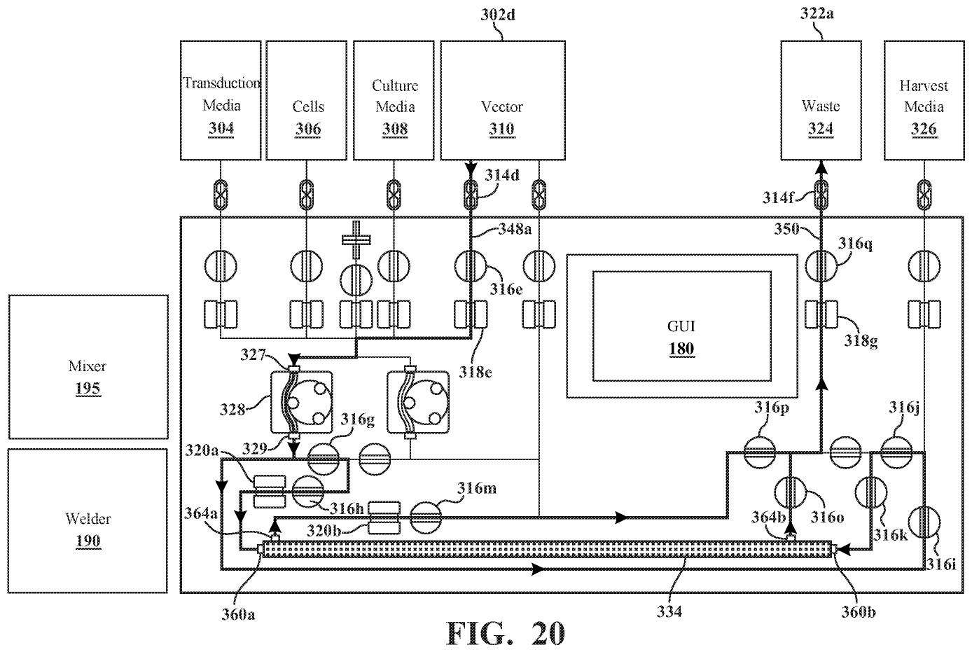

FIG. 20 illustrates execution of a ninth step 510 of the example recipe 500 for vector 310 loading into the filter module 334. In some implementations, the mixer 195 mixes the cells 306 with the vector 310 before loading the cells 306 into the filter module 334. In the example shown, the controller 200 instructs the hollow fiber system 100 to perform operations according to the example recipe 500. In the particular, the controller 200 instructs the plurality of pinch valves 316 to execute the ninth step 510 of the example recipe 500 such that pinch valves 316e, 316g, 316h, 316i, 316j, 316k, 316m, 316o, 316p, 316q are open and all other pinch valves 316 are closed. Here, a user or the controller 200 may open the flow valves 314d, 314f. Thereafter, the controller 200 instructs the first pump 328 to pump the vector 310 from the vector container 302d to the waste 324 of the waste container 322a via the intra-capillary space 338 and the extra-capillary space 339 of the filter module 334.

More specifically, the first pump 328 pumps the vector 310 into both the first intra-capillary port 360a and the second intra-capillary port 360b to create a counter-flow of the vector 310 within the intra-capillary space 338 (as shown in FIG. 3B). As the vector 310 flows from opposite ends of the intra-capillary space 338 (as shown in FIG. 3B) of the filter module 334, the counter-flow of the vectors 310 collide and/or coalesce at a common region within the intra-capillary space 338 causing the vector 310 to be captured within the filter module 334. The common region may be the same common region of the cells 306 (e.g., the seventh step 510) such that the common region defines a transduction zone. Moreover, the fluid in which the vector 310 is suspended may flow from the intra-capillary space 338 to the extra-capillary space 339 thereby exiting the extra-capillary space 339 through the first extra-capillary port 364a and the second extra-capillary port 364b through the waste conduit 350 to the waste container 322a. Notably, the vector 310 remains in the intra-capillary space 338 and only the fluid which the vector 310 is suspended in flows from the intra-capillary space 338 to the extra-capillary space 339 (e.g., as shown by cross-flow in FIG. 3B).

In this example, the bubble sensors 318a, 318g provide air quantity values to the controller 200 that represents a quantity of air in the transduction conduit 340 and the waste conduit 350 respectively. Moreover, the first fluid sensor 320a provides a pressure value and/or a fluid flow value to the controller 200 representing the pressure and/or fluid flow of vector 310 into the first intra-capillary port 360a. The second fluid sensor 320b provides a pressure value and/or a fluid value to the controller 200 representing the pressure and/or fluid flow of the vector 310 flowing from the first extra-capillary port 364a.

In some implementations, the controller 200 sequentially loads the cells 306 and the vector 310 into the filter module 334. That is, the controller 200 may first load the cells 306 into the filter module 334 and then load the vector 310 into the filter module 334 (e.g., perform the seventh step 510 and then the ninth step 510). Alternatively, the controller may first load the vector 310 into the filter module 334 and then load the cells 306 into the filter module 334 (e.g. perform the ninth step 510 and then the seventh step 510).

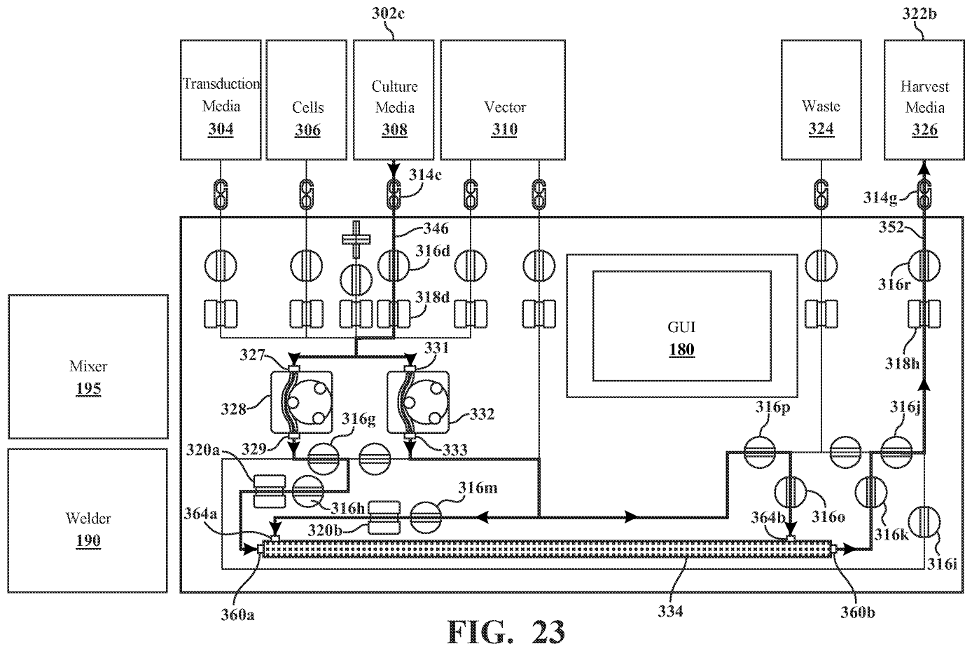

In some implementations, the controller 200 may alternate loading the cells 306 into the filter module 334 (e.g., the seventh step 510) and loading the vector 310 into the filter module (e.g., the ninth step 510). That is, loading the cells 306 into the filter module 334 may generate a first layer of cells 306 on the intra-capillary space 338 of the filter module 334. Thereafter, loading the vector 310 into the filter module 334 generates a first layer of the vector (e.g., on top of the first layer of the cells). The controller 200 may continue alternating loading the cells 306 and the vector 310 into the filter module 334 creating multiple layers of cells 306 alternating with multiple layers of the vector 310 before harvesting the cells 306 (FIG. 23). Accordingly, in a series of repeating steps in an example recipe, loading the cells 306 into the filter module 334 may correspond to a first step in a series of repeating steps in an example recipe and loading the vector 310 into the filter module corresponds to a second step in the series of repeating steps (or vice-versa). Alternating layers of cells 306 and the vector 310 may increase collisions of the cells 306 and the vector 310 during transduction.

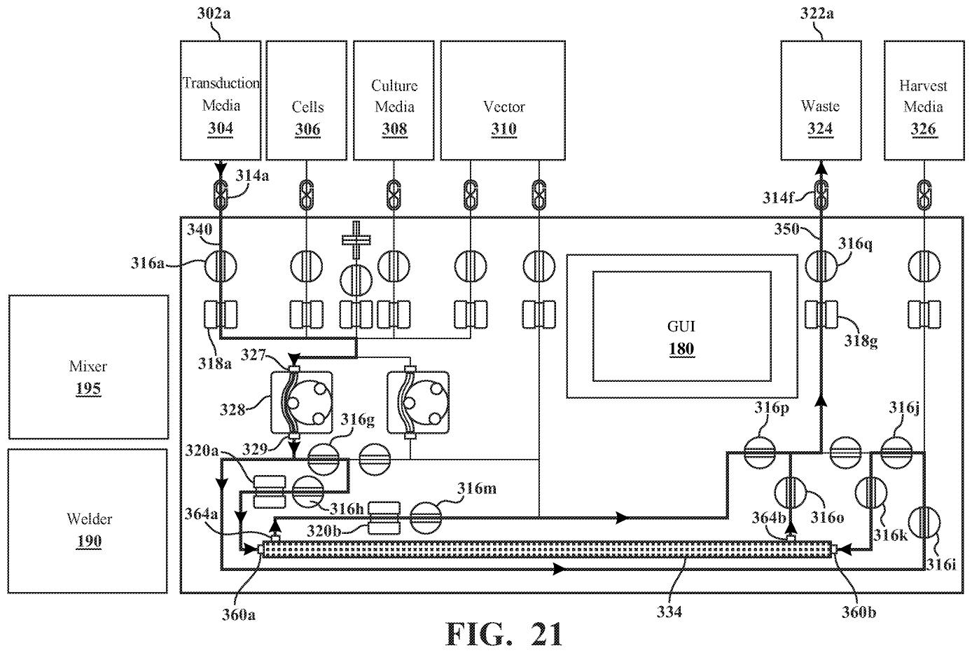

FIG. 21 illustrates execution of a tenth step 510 of the example recipe 500 for a transduction period of the cells 306 and the vector 310. That is, the tenth step 510 may be executed after execution of the ninth step 510 to promote transduction of the cells 306 and the vector 310 that are loaded into the filter module 334. In the example shown, the controller 200 instructs the hollow fiber system 100 to perform operations according to the recipe. In the particular, the controller 200 instructs the plurality of pinch valves 316 to execute the tenth step 510 of the example recipe 500 such that pinch valves 316a, 316g, 316h, 316i, 316j, 316k, 316m, 316o, 316p, 316q are open and all other pinch valves 316 are closed. Here, a user or the controller 200 may open the flow valves 314a, 314f. Thereafter, the controller 200 instructs the first pump 328 to pump the transduction media 304 from the transduction container 302a to the waste 324 of the waste container 322a via the intra-capillary space 338 and the extra-capillary space 339 of the filter module 334 (as shown in FIG. 3B). The flow of the transduction media 304 prevents diffusion and co-locates the cells 306 and the vector 310 on the surface of the filter module 334 thereby increasing transduction efficiency.

More specifically, the first pump 328 pumps the transduction media 304 into both the first intra-capillary port 360a and the second intra-capillary port 360b into the intra-capillary space 338 (as shown in FIG. 3B). The transduction media 304 flows from the intra-capillary space 338 to the extra-capillary space 339 (as shown in FIG. 3B) via the porous membrane and exits the filter module 334 through the first extra-capillary port 364a and the second extra-capillary port 364b through the waste conduit 350 to the waste container 322a. In this example, the bubble sensors 318a, 318g provide air quantity values to the controller 200 that represents a quantity of air in the transduction conduit 340 and the waste conduit 350 respectively. Moreover, the first fluid sensor 320a provides a pressure value and/or a fluid flow value to the controller 200 representing the pressure and/or fluid flow of transduction media 304 into the first intra-capillary port 360a. The second fluid sensor 320b provides a pressure value and/or a fluid value to the controller 200 representing the pressure and/or fluid flow of transduction media 304 flowing from the first extra-capillary port 364a.

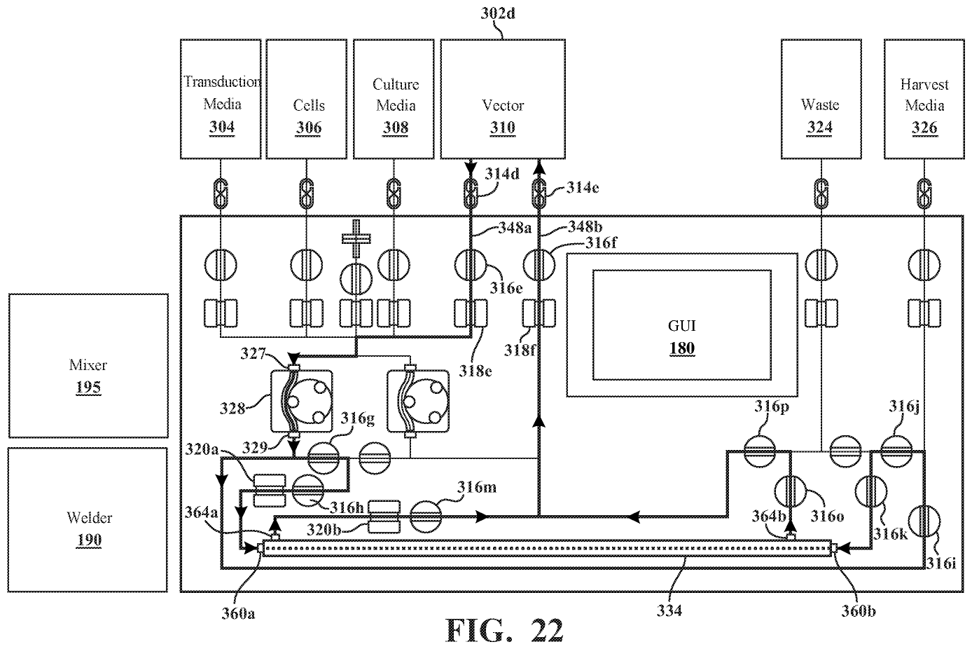

FIG. 22 illustrates execution of an eleventh step 510 of the example recipe 500 for vector 310 loading into the filter module 334 that can execute in addition to, or in lieu of, the ninth step 510 of the example recipe 500. In the example shown, the controller 200 instructs the hollow fiber system 100 to perform operations according to the example recipe 500. In the particular, the controller 200 instructs the plurality of pinch valves 316 to execute the eleventh step 510 of the example recipe 500 such that pinch valves 316e, 316f, 316g, 316h, 316i, 316j, 316k, 316m, 316o, 316p are open and all other pinch valves 316 are closed. Here, a user or the controller 200 may open the flow valves 314d, 314e.

Thereafter, the controller 200 instructs the first pump 328 to pump the vector 310 from the vector container 302d through the first virus conduit 348a into both the first intra-capillary port 360a and the second intra-capillary port 360b to create a counter-flow of the vector 310 within the intra-capillary space 338. As the vector 310 flows from opposite ends of the intra-capillary space 338 of the filter module 334, the counter-flow of the vectors 310 collide and/or coalesce at a common region within the intra-capillary space 338 causing the vector 310 to be captured within the filter module 334. The common region may be the same common region of the cells 306 (FIG. 19) such that the common region defines a transduction zone. Moreover, the fluid in which the vector 310 is suspended may flow from the intra-capillary space 338 to the extra-capillary space 339 thereby exiting the extra-capillary space 339 through the first extra-capillary port 364a and the second extra-capillary port 364b through the second virus conduit 348b to the vector container 302d. As such, the vector 310 circulates from the vector container 302d, to the filter module 334, and back to the vector container 302d thereby increasing the probability that the vector 310 will interact with the cells 306 disposed in the filter module 334.

In this example, the bubble sensors 318e, 318f provide air quantity values to the controller 200 that represents a quantity of air in the first virus conduit 348a and the second virus conduit 348b respectively. Moreover, the first fluid sensor 320a provides a pressure value and/or a fluid flow value to the controller 200 representing the pressure and/or fluid flow of vector 310 into the first intra-capillary port 360a. The second fluid sensor 320b provides a pressure value and/or a fluid value to the controller 200 representing the pressure and/or fluid flow of the vector 310 flowing from the first extra-capillary port 364a.

FIG. 23 illustrates execution of a twelfth step 510 of the example recipe 500 for a harvest period where the harvest container 322b recovers cells 306 from the filter module 334 after the transduction period. In the example shown, the controller 200 instructs the hollow fiber system 100 to perform operations according to the example recipe 500. In the particular, the controller 200 instructs the plurality of pinch valves 316 to execute the twelfth step 510 of the example recipe 500 such that pinch valves 316d, 316g, 316j, 316k, 316m, 316o, 316p, 316r are open and all other pinch valves 316 are closed. Here, a user or the controller 200 may open the flow valves 314c, 314g. Thereafter, the controller 200 instructs the first pump 328 and the second pump 332 to pump the culture media 308 from the culture container 302c to the harvest media 326 of the harvest container 322b via the intra-capillary space 338 and the extra-capillary space 339 of the filter module 334.

More specifically, the first pump 328 pumps the culture media 308 into the first intra-capillary port 360a through the intra-capillary space 338 and out the second intra-capillary port 360b to the harvest container 322b through the harvest conduit 352. Moreover, the second pump 332 pumps the culture media 308 into the first extra-capillary port 364a and the second extra-capillary port 364b. Thus the culture media 308 flows from the extra-capillary space 339 into the intra-capillary space 338 through the porous membrane. As such, the culture media 308 exits the filter module 334 through the second extra-capillary port 364b to the harvest conduit 352 and finally to the harvest container 322b. Notably, here the fluid flow direction of the culture media 308 flow from the extra-capillary ports 364 into the intra-capillary space 338 of the filter module 334. This reverse fluid flow lifts cells 306 from the membrane into the intra-capillary space 338. Moreover, the flow from the first intra-capillary port 360a to the second intra-capillary port 360b drives the cells 306 into the harvest container 322b.

In this example, the bubble sensors 318d, 318h provide air quantity values to the controller 200 that represents a quantity of air in the culture conduit 346 and the harvest conduit 352, respectively. Moreover, the first fluid sensor 320a provides a pressure value and/or a fluid flow value to the controller 200 representing the pressure and/or fluid flow of culture media 308 into the first intra-capillary port 360a. The second fluid sensor 320b provides a pressure value and/or a fluid value to the controller 200 representing the pressure and/or fluid flow of the culture media 308 flowing into the first extra-capillary port 364a.

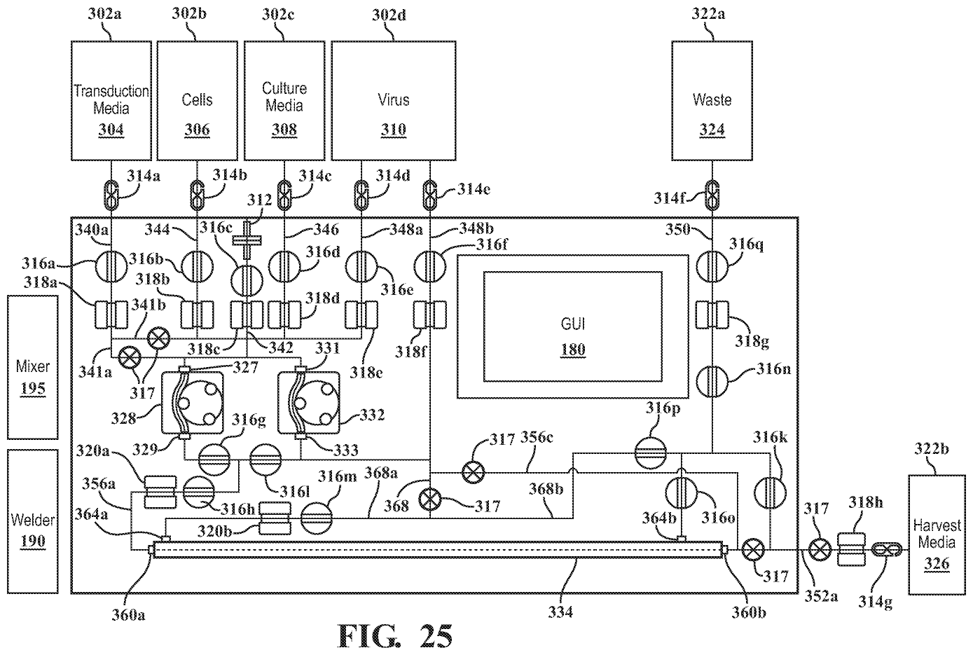

With particular reference to FIG. 25, a schematic of another example of a hollow fiber system 100a is provided. In view of the substantial similarity in structure and function of the components associated with the hollow fiber system 100 with respect to the hollow fiber system 100a, like reference numerals are used hereinafter and in the drawings to identify like components while like reference numerals containing letter extensions are used to identify those components that have been modified or provided in multiple instances.

While the hollow fiber system 100a shown in FIG. 25 includes common components with the hollow fiber system 100 described above, the hollow fiber system 100a has been modified to include alternate flow paths and valves configured to route the input materials 304, 306, 308, 310 through the hollow fiber system 100a. For example, a transduction conduit 340a of the hollow fiber system 100a branches into a first transduction subconduit 341a and a second transduction subconduit 341b. Here the first transduction subconduit 341a provides a direct fluid path from the transduction container 302a to the first pump inlet 327, while the second transduction subconduit 341b is connected with each of the conduits 342, 344, 346, 348 upstream of the first pump inlet 327 and the second pump inlet 331. Thus, the first transduction subconduit 341a and the second transduction subconduit 341b provide parallel and alternative flow paths between the transduction container 302a and at least the first pump 328. Each of the first transduction subconduit 341a and the second transduction conduit 341b may include one or more valves 317. In FIG. 25, each valve 317 is generically represented. However, the one or more valves 317 may include flow valves 314, pinch valves 316, and/or other types of valves for controlling flow through the transduction subconduits 341a, 341b. Optionally, each of the transduction subconduits 341a, 341b may include flow sensors, such as a bubble sensor 318.

With continued reference to FIG. 25, the hollow fiber system 100a is provided with an alternative configuration of the second intra-capillary conduit 356c. While the second intra-capillary conduit 356b described above connects the first pump outlet 329 to the second intra-capillary port 360b, the second intra-capillary conduit 356c of the hollow fiber system 100a provides a direct fluid flow path from the second pump outlet 333 to the second intra-capillary port 360b. The second intra-capillary conduit 356c may include one or more of the valves 317, which may include the flow valves 314 and or the pinch valves 316. Optionally, the second intra-capillary conduit 356c may include flow sensors, such as a bubble sensor 318.

With the alternative configuration of the second intra-capillary conduit 356c, the extra-capillary conduit 368 may include one or more valves 317 to selectively control flow to both of the first extra-capillary subconduit 368a and the second extra-capillary subconduit 368b. However, in some examples, the valve 317 may be omitted from the extra-capillary conduit 368, whereby flow through each of the extra-capillary subconduits 368a, 368b is independently controlled via the corresponding pinch valves 316m, 316o, 316p.

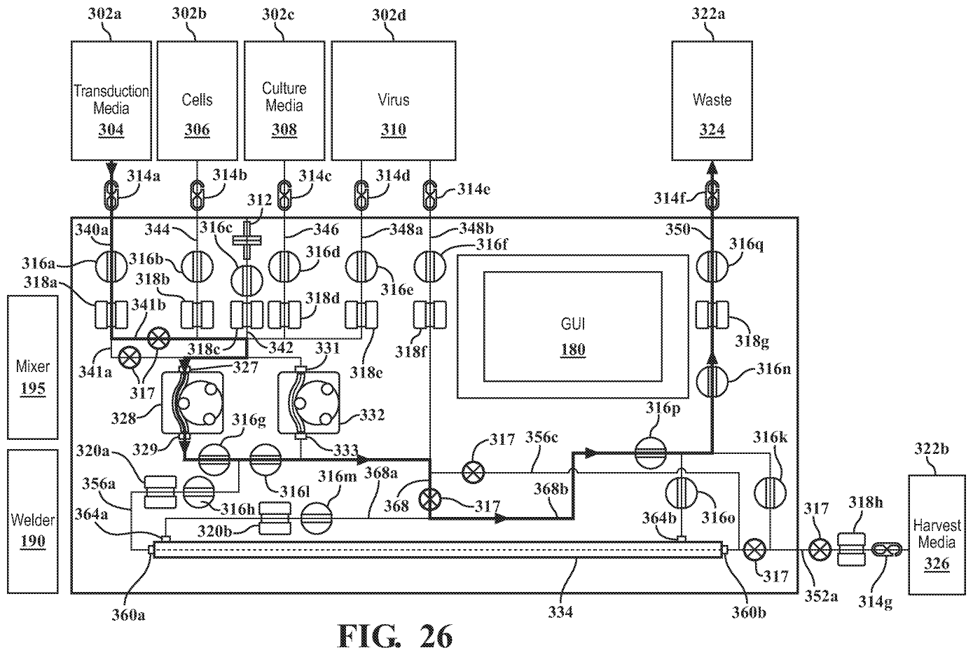

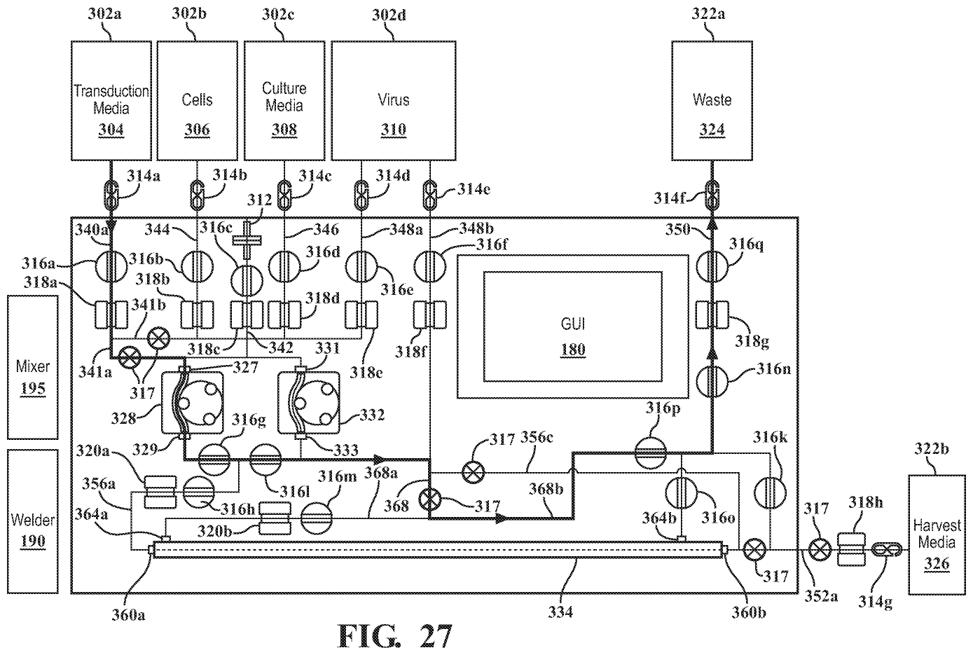

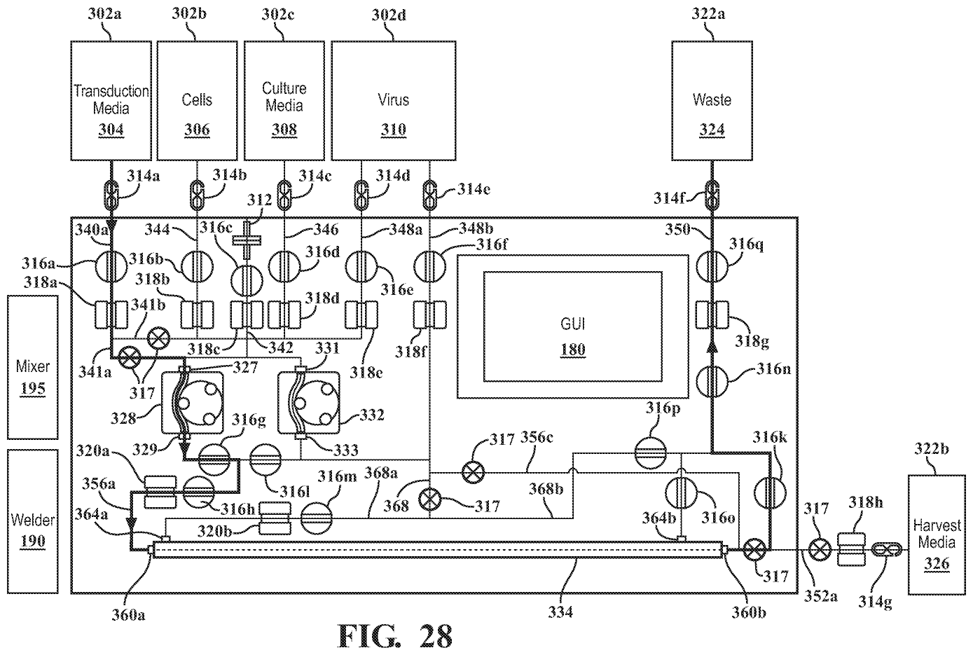

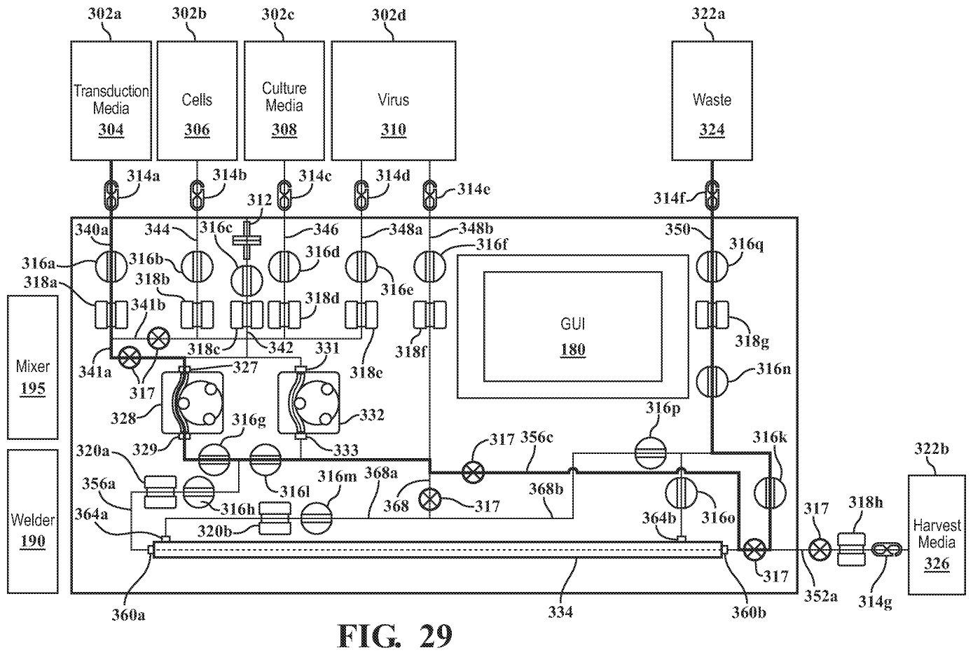

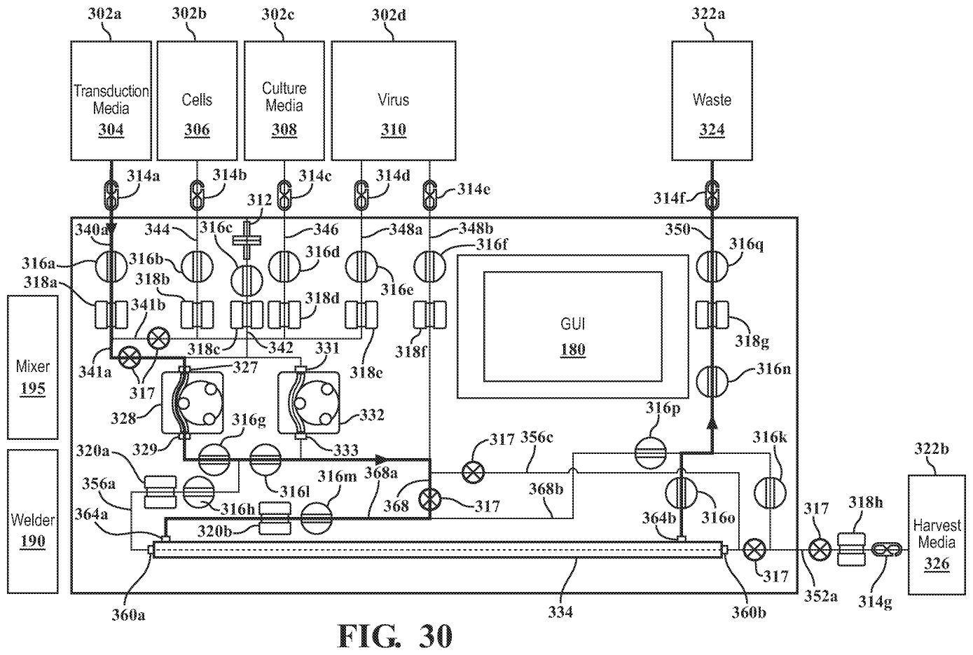

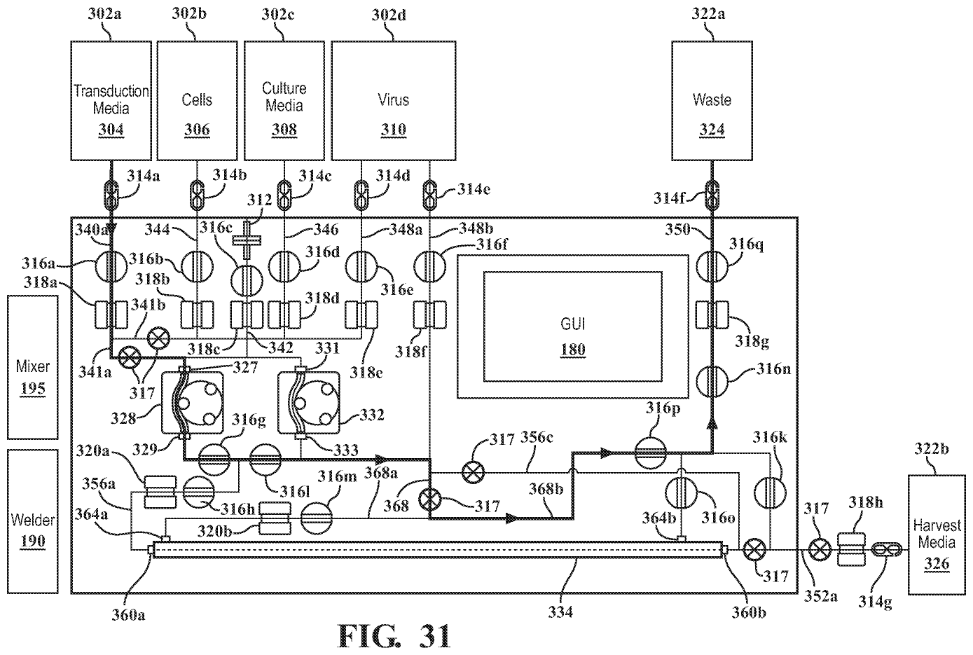

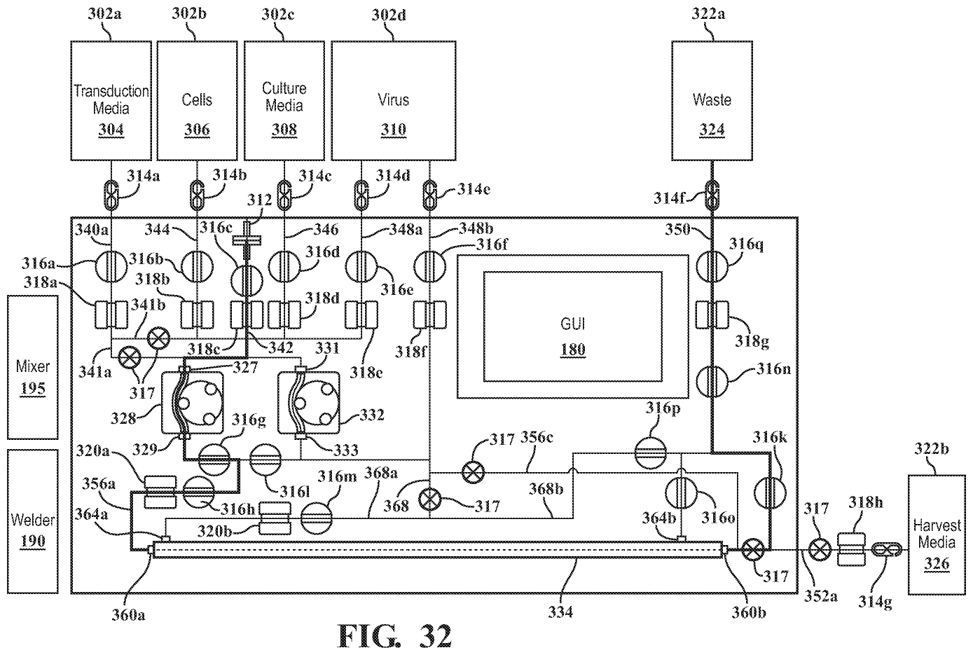

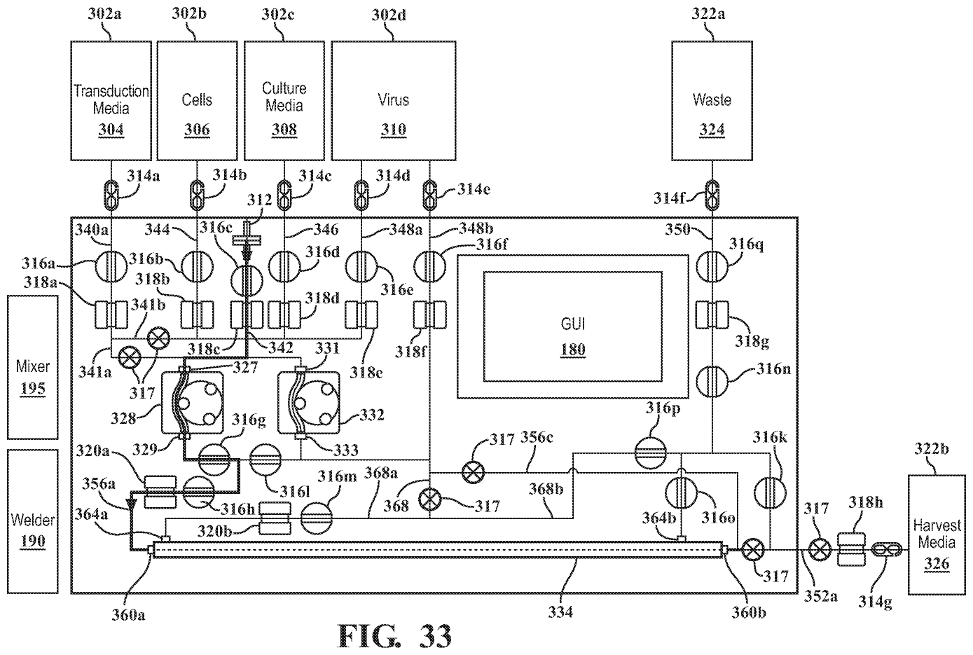

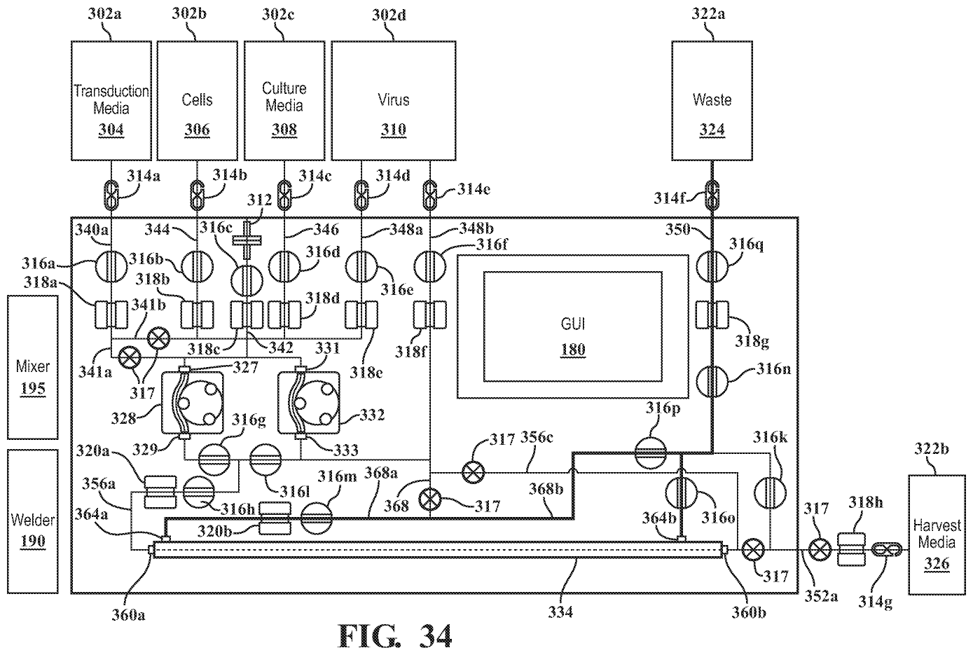

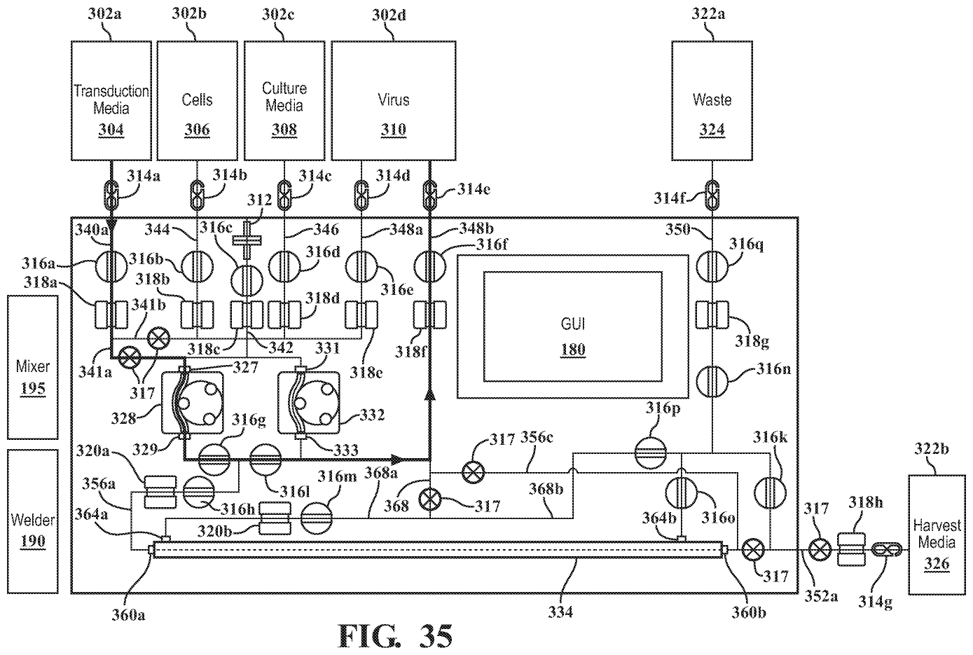

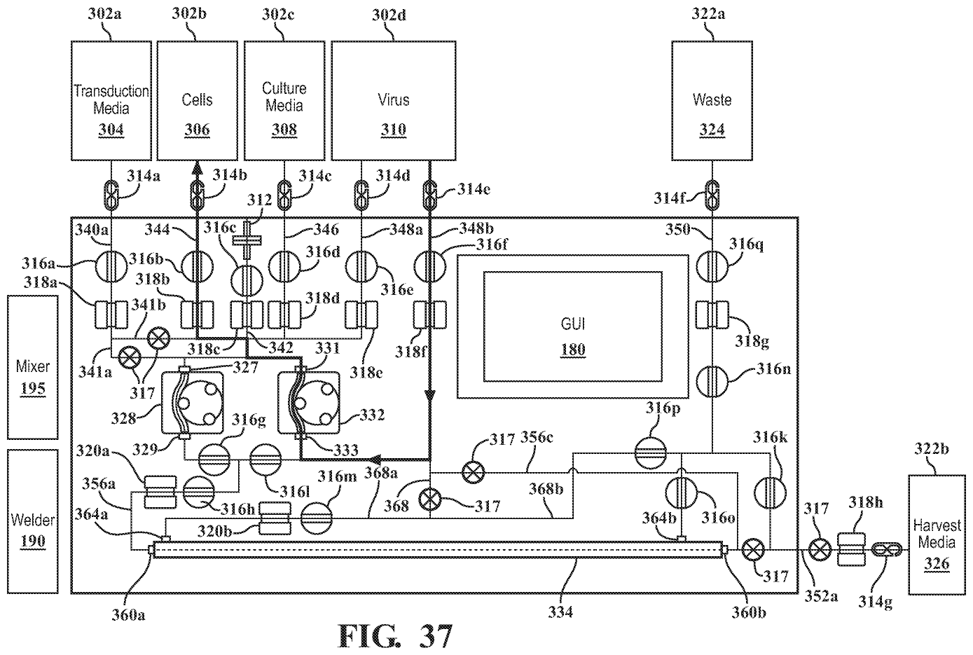

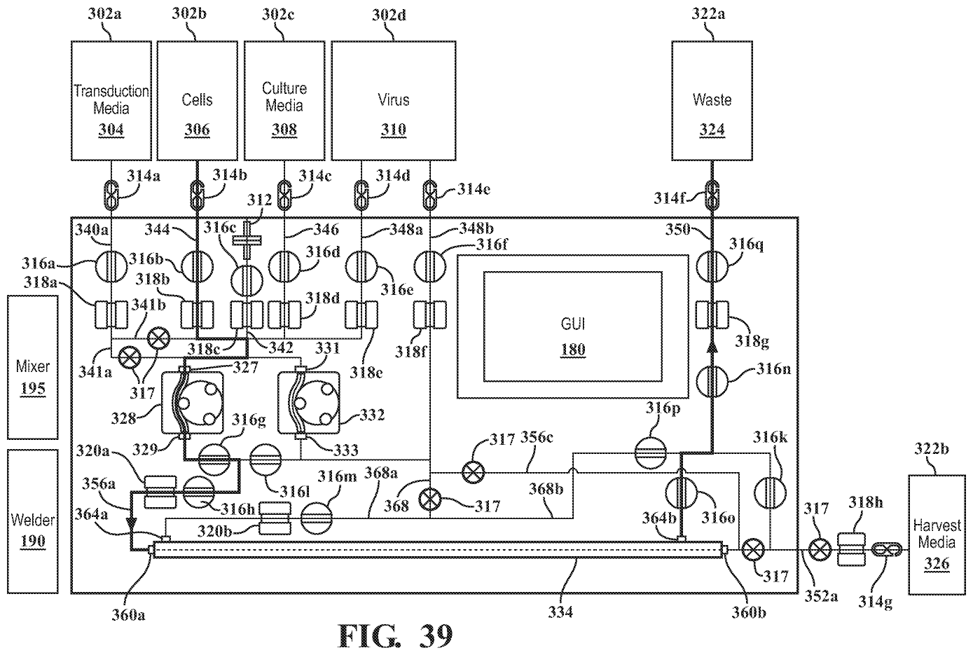

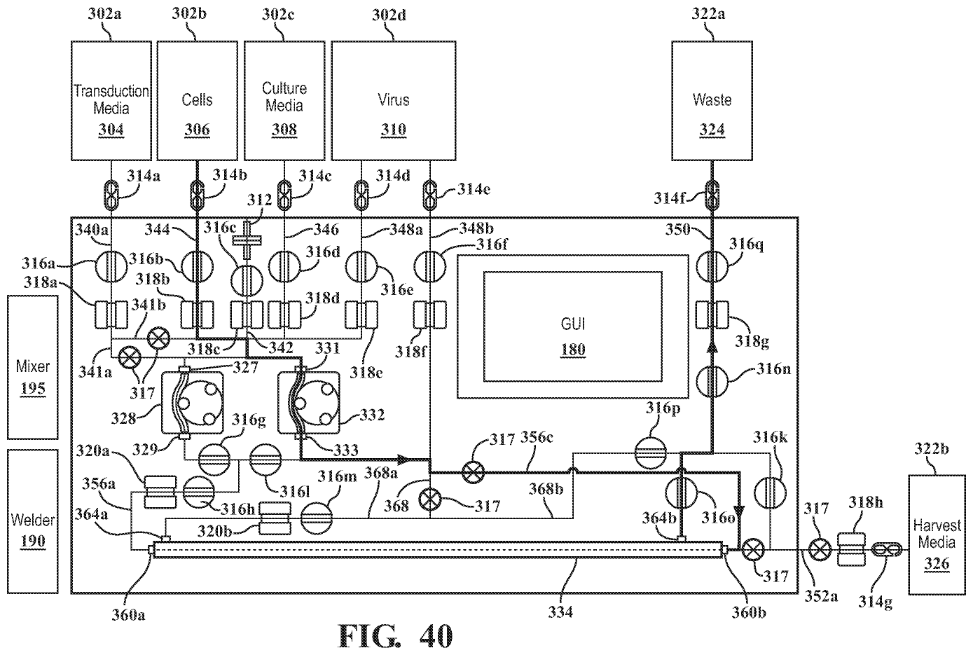

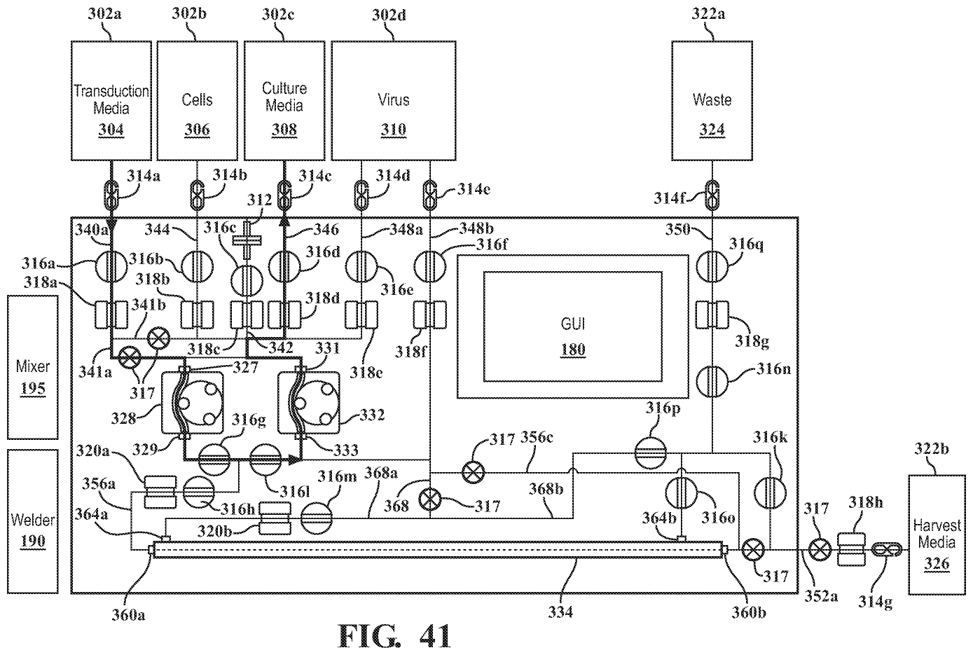

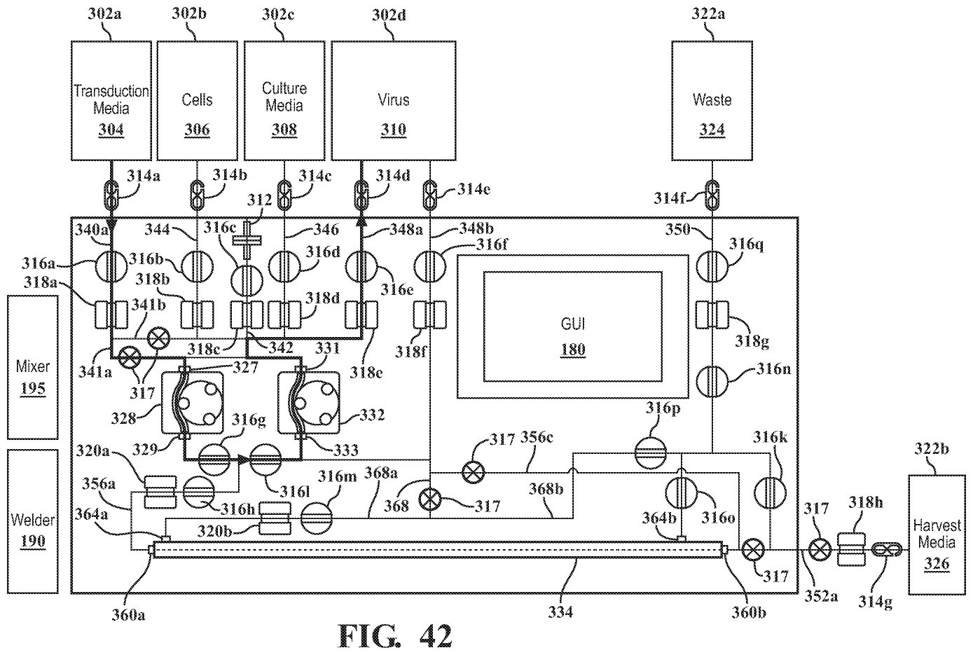

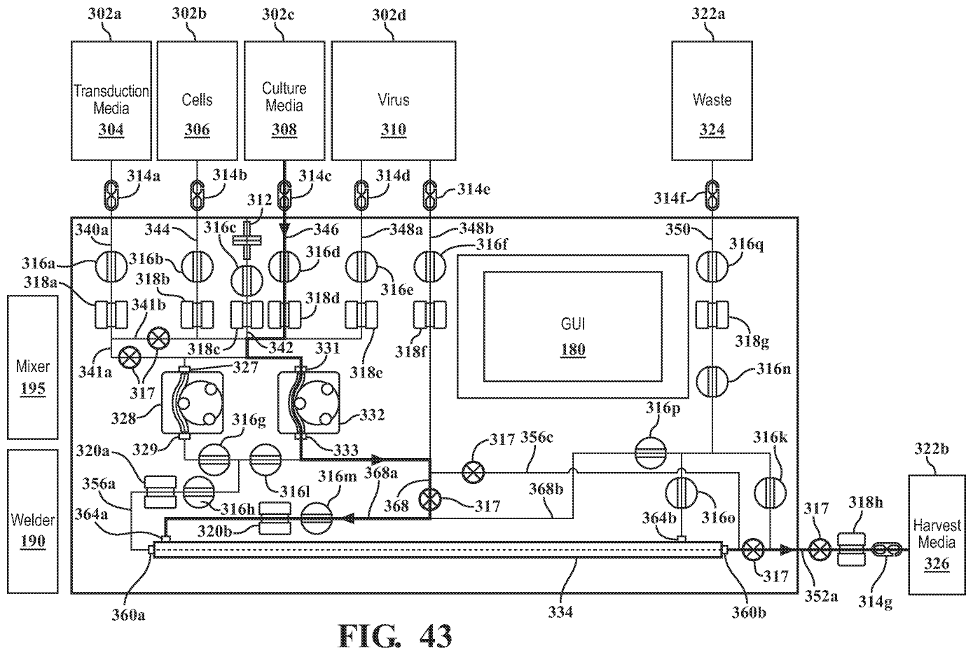

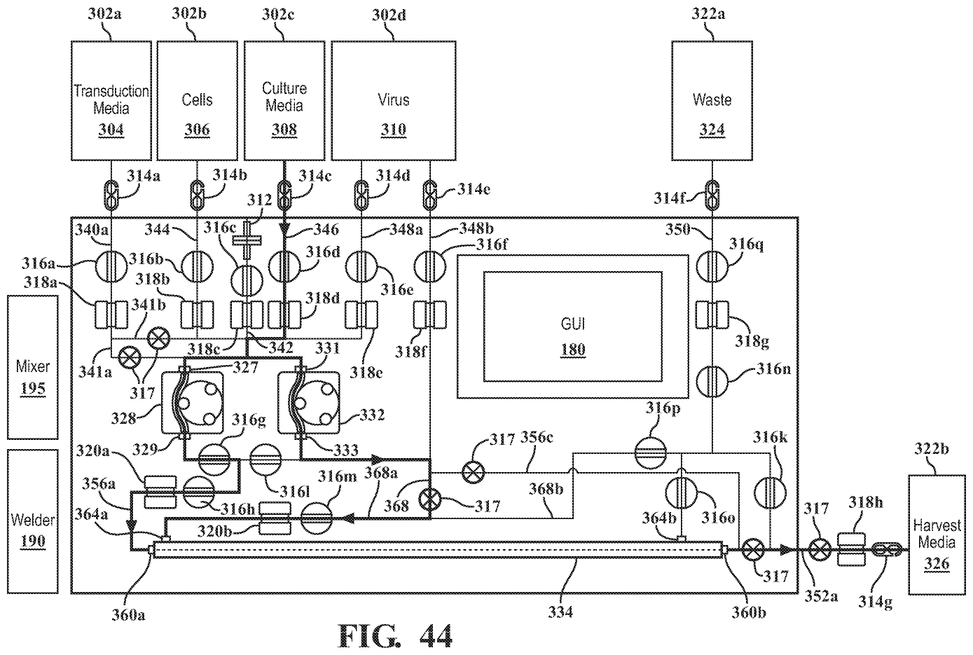

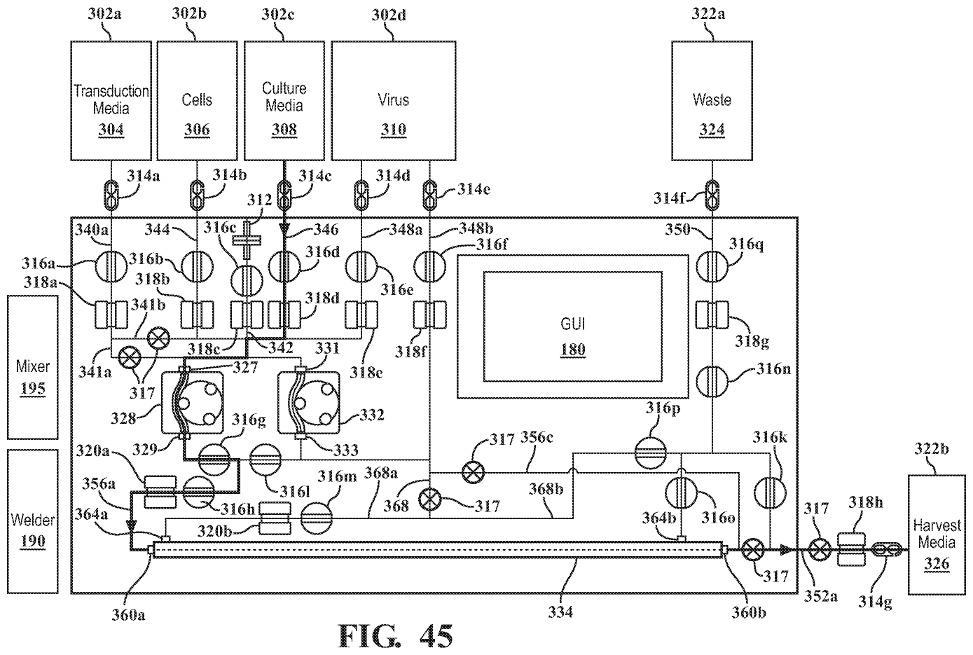

FIGS. 26-46 illustrate example schematic views of fluid flow paths through the hollow fiber system 100a according to a plurality of steps 510 of a recipe 500. In particular, each figure in FIGS. 26-46 illustrates one step 510 from the plurality of steps 510 of the recipe 500. It is understood that the plurality of steps 510 as shown in FIGS. 26-46 illustrate an example recipe only and are not meant to limit this disclosure in any way. For example, a different example recipe may repeat any of the plurality of steps any number of times, include more or fewer steps, and/or perform the plurality of steps in a different order than the example recipe illustrated in FIGS. 26-46.

It is understood that FIGS. 26-46 include the same components as FIG. 25, however, for the sake of clarity, all of the components of the system 100a are labeled in FIG. 26. In these examples, the direction of arrows indicates fluid flow in the hollow fiber system 100a. Moreover, bold lines represent which conduits have active fluid flow paths and non-bold lines represent conduits without active fluid flow paths. Notably, non-bold lines may still have fluid within, but the fluid is not actively flowing.

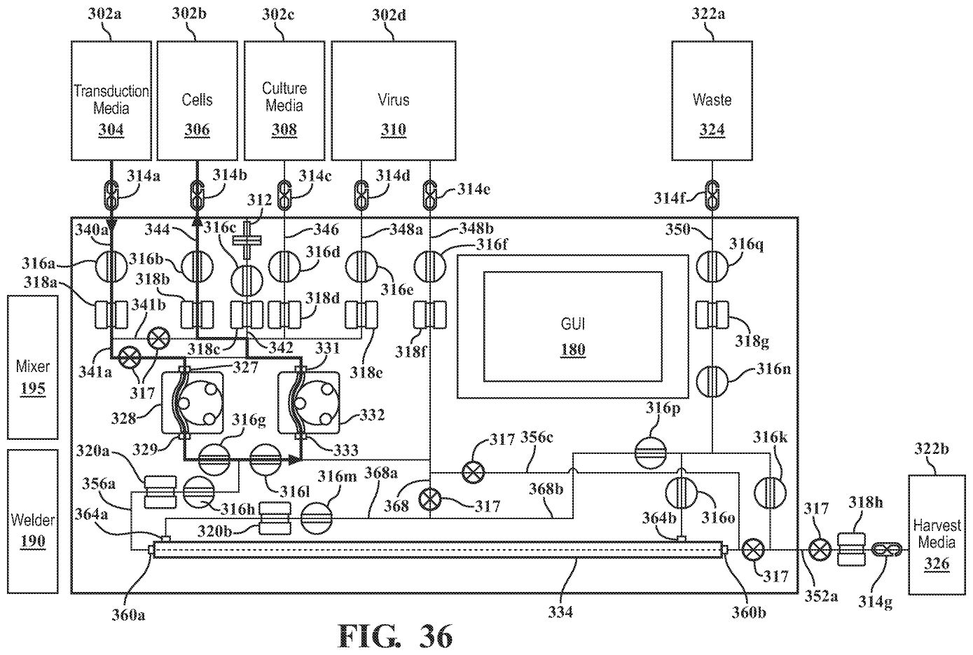

The configuration of the hollow fiber system 100a provided in FIGS. 25-46 provides different functionalities relative to the hollow fiber system 100 set forth in FIGS. 3 and 12-24. For example, by providing the first transduction subconduit 341a and the second intra-capillary conduit 356c, the hollow fiber system 100a is operable to prime all of the components of the system with the transduction media 304, as shown in FIGS. 26-31, 35, and 36. This ensures that clean transduction media 304 is always flowing through the conduits, rather than relying on backward flow of waste 324 from the waste container 322a. For example, compare the execution of the sixth step 510 for priming the cell conduit 344 shown in FIG. 17 relative to the priming of the cell conduit 344 shown in FIG. 36. In FIG. 17, the cell conduit 344 is primed using waste 324 from the waste container 322, while in FIG. 36 the cell conduit 344 is primed using the transduction media 304 (i.e., a priming media) via the first transduction subconduit 341a.

Providing a forward flow of the transduction media 304 to prime the cell flow path rather than utilizing a backflow of the waste 324 is particularly helpful in ensuring compliance with Good Manufacturing Practices (GMP), as the transduction media 304 will remain free of any contaminants that may be flushed from the system 100a into the waste container 322a during any of the priming steps. For example, the filter module 334 may be initially provided from the manufacturer with a sugar substrate that is included during shipment and storage of the filter module 334 to stabilize the pores. During the priming steps, the sugar substrate is flushed from the filter module 334 to prepare the filter module 334 for use. By only utilizing the transduction media as a priming solution, the system 100a eliminates the potential of any of the sugar substrate that may be contained in the waste 324 from being introduced to the cell container 302b during the cell conduit 344 priming step.

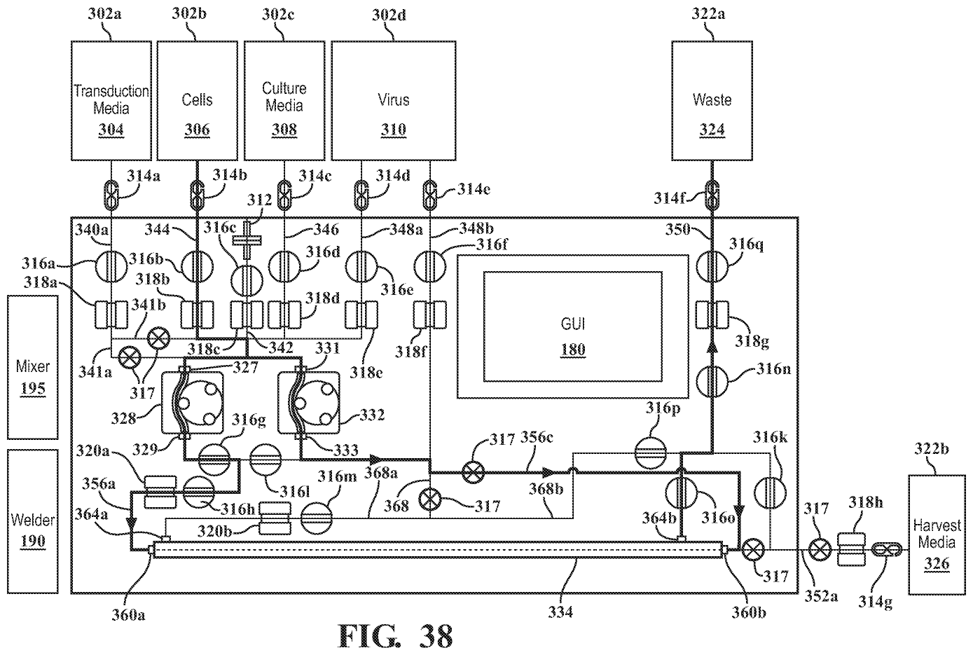

In addition to providing a different priming configuration, the hollow fiber system 100a provides more direct flow paths between the input containers 302 and the second intra-capillary port 360b by providing the second intra-capillary conduit 356c. Particularly, as shown in FIG. 38, the inclusion of the second intra-capillary conduit 356c allows cells 306 to be delivered directly to the second intra-capillary port 360b via the second pump 332. Thus, the intra-capillary ports 360a, 360b can be independently supplied with cells 306 by a respective one of the pumps 328, 332 rather than relying on the first pump 328 to provide the cells 306 to both of the intra-capillary ports 360a, 360b (see e.g., FIG. 18). This configuration advantageously improves system efficiency and provides component redundancy within the hollow fiber system 100a.

While the implementation of the hollow fiber system 100a is represented schematically in FIGS. 25-45, whereby arrangements of components are generally provided relative to the hollow fiber system 100 shown in FIG. 1A, the configuration of the hollow fiber system 100a advantageously allows the lengths of several of the conduits to be minimized relative to the lengths of such conduits in the system 100. For instance, by implementing the second intra-capillary conduit 356c flowing directly from the second pump 332, the overall length of the second intra-capillary conduit 356c is reduced relative to the length of the second intra-capillary conduit 356b of the hollow fiber system 100. Likewise, reconfiguring the intra-capillary conduit 356c allows the pinch valve 316j (see FIG. 18) to be removed from the system 100a. By eliminating this pinch valve 316j, the harvest conduit 352a can be shortened relative to the length of the harvest conduit 352 provided in the system 100. Designing the system 100a to minimize the lengths of respective conduits advantageously eliminates low-flow or stagnant regions within the system 100a, which, in turn, minimizes the likelihood of cells becoming stuck within the conduits and/or settling outside of the filter module 334 during cell loading steps of the recipe 500.

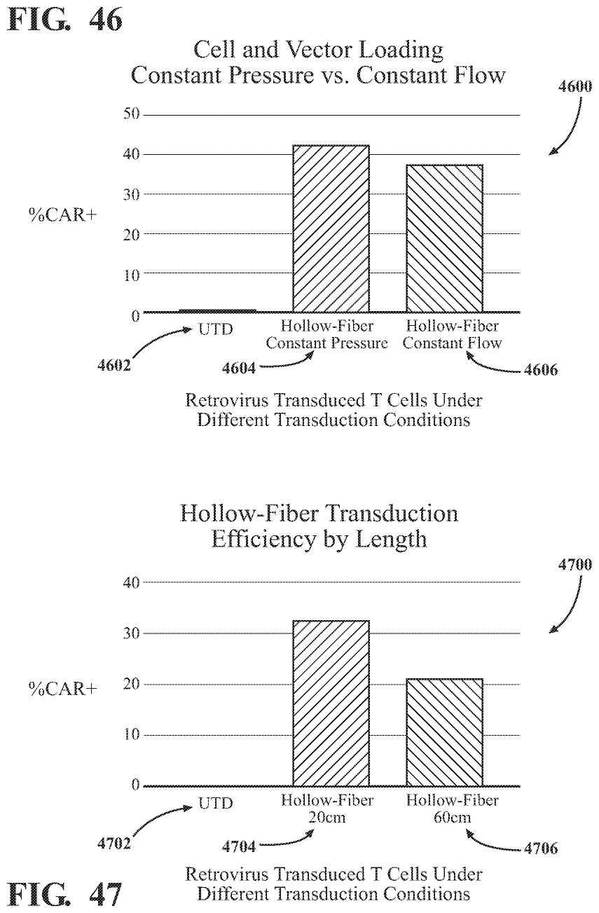

FIG. 46 illustrates a chart 4600 of optimization studies in a fully-automated hollow fiber system 100. The chart 4600 demonstrates retrovirus transduction of the cells 306 in the hollow fiber system 100. In this instance, the cells 306 are αβT cells. The chart 4600 illustrates a comparison of the results of a first condition 4602, a second condition 4604, and a third condition 4606 for loading the cells 306 and vector 310 in the filter module 334. The first condition 4602 includes untransduced cells. The second condition 4604 includes cells 306 and vectors 310 loaded into the filter module 334 under a constant pressure, and the third condition 4606 includes cells 306 and vectors 310 loaded into the filter module 334 under a constant flow rate. The second condition 4604 is configured such that the hollow fiber system 100 adjusts the flow rate of the pump 328, 332 to maintain a target fluid pressure. The third condition 4606 is configured such that the fully-automated hollow fiber system 100 is configured to maintain the flow rate of the pump 328, 332 at a pre-set speed. For instance, in the third condition 4606, the hollow fiber system 100 may only adjust the flow rate of the pump 328, 332 if the hollow fiber system 100 reaches or exceeds a threshold pressure setting.

FIG. 47 illustrates a chart 4700 of optimization studies in a fully-automated platform of the hollow fiber system 100. The chart 4700 demonstrates retrovirus transduction of αβT cells 306 in the hollow fiber system 100. The chart 4700 illustrates a comparison of the results associated with a first condition 4702, a second condition 4704, and a third condition 4706 for cell 306 and vector 310 loading. The first condition 4702 includes untransduced cells 306. The second condition 4704 includes cells 306 and vectors loaded at a concentration of approximately 1 mol (M) cells/cm² into approximately 20 centimeters length of hollow fiber. The third condition 4706 includes cells 306 and vectors 310 loaded at a concentration of approximately 1M cells/cm² into a 40 centimeter length hollow-fiber. In operation, the flow rates, cell numbers, and cell volumes were normalized across the lengths of the hollow fibers in the second and third conditions 4704, 4706 based on the relative difference of the surface area across the two fibers.

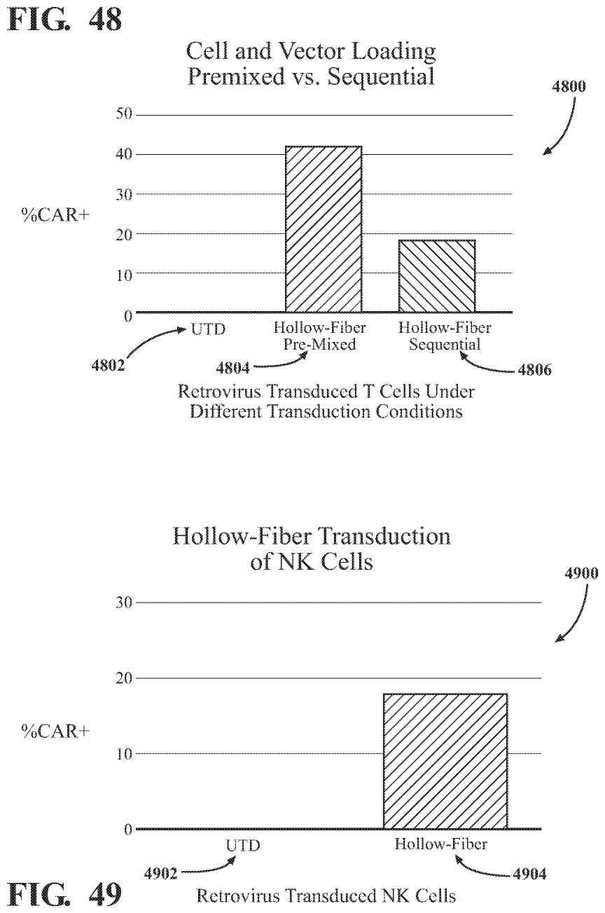

FIG. 48 illustrates a chart 4800 of optimization studies in a fully-automated hollow fiber system 100. The chart 4800 demonstrates retrovirus transduction of αβT cells 306 in the hollow fiber system 100. The chart 4800 illustrates a comparison of results of associated with a first condition 4802, a second condition 4804, and a third condition 4806 for cell 306 and vector 310 loading in the filter module 334. The first condition 4802 includes untransduced cells 306. The second condition 4804 includes premixing the cells 306 and vector 310 on the hollow fiber system 100 before loading the mixture into the filter module 334. The third condition 4800 includes sequentially loading the cells 306 and the vector 310 into the filter module 334. In the third condition 4806, the cells 306 are loaded into the hollow fiber system 100 first and the vector is loaded second.

Under the second condition 4804, the fully-automated hollow fiber system 100 pumps the vector 310 from a vector transfer bag 302 into a transfer bag 302 containing the cell suspension 306. The cells 306 are then mixed by hand by an operator before the cells 306 and vector 310 mixture solution is loaded into the filter module 334. Under the third condition 4806, the cells 306 and the vector 310 remain separate during the loading steps and are only co-localized within the filter module 334. It is contemplated that the flow rates are kept constant across the second and third conditions 4804, 4806.

Each of the charts 4600, 4700, and 4800 illustrate an example gamma retrovirus vector 310 that uses a Chimeric Antigen Receptor (CAR) construct. For example, CD4/CD8 isolated T cells are thawed and activated for approximately 48 hours. CD4 T cells are a type of white blood cell that assist in fighting infections, and CD8 T cells assist in eliminating virus-infected cells. The operations in obtaining the results depicted in the charts 4600, 4700, 4800 include the following operations. After thawing and activating the cells 306, the cells 306 and the vector 310 are moved into separate transfer bags 302, and the transfer bags 302 are sterile-welded onto the hollow fiber system 100. In further operation, a pre-defined program may be initiated on the computing device 200 of the hollow fiber system 100 to introduce the cells 306 and vector 310 under either constant pressure or constant flow conditions. Both of the conditions are transduced at a multiplicity of infection (MOI) of approximately five. It is contemplated that in alternate operations the MOI may be greater than five or less than five. Once the cells 306 and the vector 310 are introduced, the hollow fiber system 100 automatically shifts to a ninety-minute transduction stage followed by a harvest operation to recover the cells 306 from the filter module 334. The recovered cells 306 are then cultured for an additional period (approximately three to four days) before being analyzed for the percentage of CAR expression using flow cytometry.

FIG. 49 illustrates a chart 4900 of optimization studies in a fully automated hollow fiber system 100. The chart 4900 demonstrates results of a retrovirus transduction of NK cells 306 in the filter module 334. The chart 4900 illustrates a comparison of results associated with a first condition 4902 and a second condition 4904 for cell 306 and vector 310 loading in the filter module 334. The first condition 4902 includes untransduced cells 306 and the second condition 4904 includes NK cells 306 transduced on the fully-automated hollow fiber system 100.

In this example, the vector 310 may be a gamma retrovirus vector using a CAR construct. In operation, CD56 NK cells 306 are negatively isolated and activated for approximately six days. CD56 is a phenotypic marker of the NK cells. The cells 306 and vector 310 may then be moved into separate transfer bags 302, which are sterile-welded onto the hollow fiber system 100. A predefined program may then be initiated on the computing device 200 of the hollow fiber system 100 to either introduce the cells 306 and vector 310 under constant pressure or constant flow conditions. As mentioned above, both conditions are transduced at a MOI of approximately five. It is contemplated that in alternate operations the MOI may be greater than five or less than five. Following introduction of the cells 306 and vector 310, the platform may automatically move to a ninety-minute transduction stage followed by a harvest step to recover the cells 306 from the filter module 334. The recovered cells 306 may be reactivated and cultured for an additional period (approximately fourteen days) before being analyzed for the percentage of CAR expression using flow cytometry.

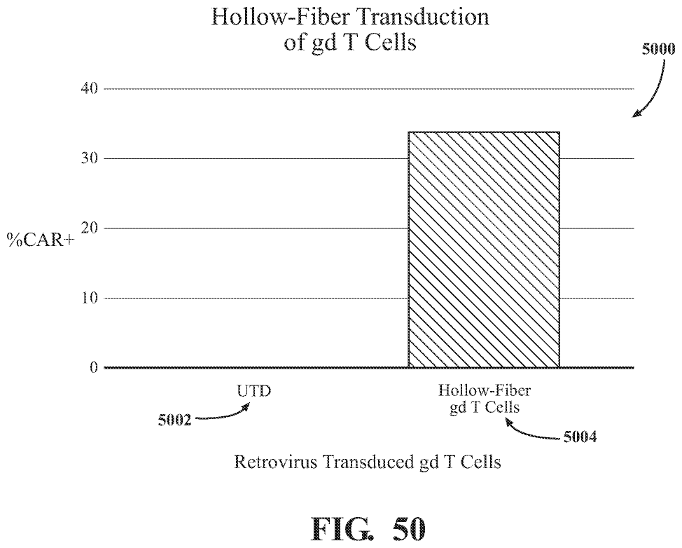

FIG. 50 illustrates a chart 5000 of optimization studies in a fully automated the hollow fiber system 100. The chart 5000 demonstrates results of a retrovirus transduction of γδT cells 306 in the filter module 334. The chart 5000 illustrates a comparison of results associated with a first condition 5002 and a second condition 5004 for cell 306 and vector 310 loading in the filter module 334. The first condition 5002 includes untransduced cells 306, and the second condition 5004 includes γδT cells 306 transduced on the fully-automated hollow fiber platform 100.

In this example, the vector 310 may be a gamma retrovirus vector using a CAR construct. In operation, γδT cells 306 are negatively isolated and activated for approximately six days. The cells 306 and vector 310 may then be moved into separate transfer bags 302, which are sterile-welded onto hollow fiber system 100. A predefined program may then be initiated on the computing device 200 of the hollow fiber system 100 to either introduce the cells 306 and vector 310 under constant pressure or constant flow conditions. Both conditions are transduced at a MOI of approximately 7.5. It is contemplated that in alternate operations the MOI may be greater than 7.5 or less than 7.5. Following introduction of the cells 306 and vector 310, the platform may automatically move to a ninety-minute transduction stage followed by a harvest step to recover the cells 306 from the filter module 334. The recovered cells 306 may be reactivated and cultured for an additional period (approximately ten days) before being analyzed for the percentage of CAR expression using flow cytometry.

A number of implementations have been described. Nevertheless, it will be understood that various modifications may be made without departing from the spirit and scope of the disclosure. Accordingly, other implementations are within the scope of the following claims.

1-16. (canceled)

17. A system comprising:
a filter module defining an intra-capillary space and an extra-capillary space separated from the intra-capillary space by a porous membrane, the filter module including a pair of intra-capillary ports fluidly coupled to the intra-capillary space and a pair of extra-capillary ports coupled the extra-capillary space;
a first pump including a first pump inlet in communication with each of a cell source including cells and a vector source including a vector, and a first pump outlet in selective communication with each of the pair of intra-capillary ports and the pair of extra-capillary ports;
a second pump including a second pump inlet in communication with each of the cell source and the vector source, and a second pump outlet in selective communication with each of the pair of intra-capillary ports and the pair of extra-capillary ports; and
a graphical user interface (GUI) in operative communication with each of the first pump and the second pump and configured to receive a request from a user for operating the first pump and second pump according to a recipe comprising one or more steps.

18. The system of claim 17, further comprising:
at least one valve disposed between the first pump outlet and a first intra-capillary port of the pair of intra-capillary ports;
at least one valve disposed between the first pump outlet and a second intra-capillary port of the pair of intra-capillary ports; and
a fluid sensor disposed between the first pump outlet and the first intra-capillary port of the pair of intra-capillary ports,
wherein the valves operate according to the recipe.

19. The system of claim 17, further comprising:
a valve disposed between the first pump inlet and the cell source; and
a bubble sensor disposed between the first pump inlet and the cell source,
wherein the valve operates according to the recipe.

20. The system of claim 17, further comprising:
at least one valve disposed between the second pump outlet and a first intra-capillary port of the pair of extra-capillary ports;
at least one valve disposed between the second pump outlet and a second intra-capillary port of the pair of intra-capillary ports; and
a fluid sensor disposed between the second pump outlet and the first intra-capillary port of the pair of extra-capillary ports,
wherein the valves operate according to the recipe.

21. The system of claim 17, wherein the GUI is further configured to, in response to receiving the request, display to the user the recipe from memory hardware.

22. A system comprising:
a filter module defining an intra-capillary space and an extra-capillary space separated from the intra-capillary space by a porous membrane, the filter module including a pair of intra-capillary ports fluidly coupled to opposite ends of the intra-capillary space and a pair of extra-capillary ports coupled to opposite ends of the extra-capillary space;
a first pump including a first pump inlet in communication with each of a cell source including cells and a vector source including a vector, and a first pump outlet in selective communication with a first intra-capillary port of the pair of intra-capillary ports and a second intra-capillary port of the pair of intra-capillary ports;
a first fluid sensor disposed between the first pump outlet and the first intra-capillary port of the pair of the intra-capillary ports;
data processing hardware; and
memory hardware in communication with the data processing hardware, the memory hardware storing instructions that when executed on the data processing hardware cause the data processing hardware to perform operations according to a recipe for transducing the cells with the vector.

23. The system of claim 22, wherein the operations comprise:
operating the first pump at a first pump rate to provide a first flow of at least one of the cells or the vector to each of the pair of the intra-capillary ports of the filter module;
obtaining, from the first fluid sensor, a first fluid flow signal indicating the first flow of at least one of the cells or the vector;
comparing the first fluid flow signal against a first fluid flow parameter threshold; and
operating the first pump at a second pump rate depending on the comparison.

24. The system of claim 23, wherein the operations further comprise initiating a pump rate adjustment procedure depending on the comparison, the pump rate adjustment procedure comprising stopping operation of the first pump for a predetermined period.

25. The system of claim 24, wherein the predetermined period is a period of time.

26. The system of claim 24, wherein the predetermined period is a pressure adjustment period.

27. The system of claim 24, wherein the pump rate adjustment procedure further comprises operating the first pump in a reverse-flow state.

28. The system of claim 23, wherein the operations further comprise operating the first pump at a third pump rate to provide a second flow of the other of the one of the cells or the vector to each of the pair of the intra-capillary ports of the filter module.

29. The system of claim 28, wherein the operations further comprise instructing the first pump to alternate operation between:
operating at the first pump rate to provide the first flow of the at least one of the cells or the vector to each of the pair of the intra-capillary ports of the filter module; and
operating at the third pump rate to provide the second flow of the other of the one of the cells or the vector to each of the pair of the intra-capillary ports of the filter module.

30. The system of claim 28, wherein the operations further comprise operating the first pump at the third pump rate to provide the second flow of the other of the one of the cells or the vector to each of the pair of the intra-capillary ports of the filter module after operating the first pump at the first pump rate to provide the first flow of the at least one of the cells or the vector to each of the pair of the intra-capillary ports of the filter module.

31. The system of claim 28, wherein the operations further comprise:
obtaining, from the first fluid sensor, a second fluid flow signal indicating the second flow of the other of the one of the cells or the vector;
comparing the second fluid flow signal against a second fluid flow parameter threshold; and
initiating a pump rate adjustment procedure depending on the comparison.

32. The system of claim 22, further comprising a transduction media source including transduction media in communication with the first pump inlet, wherein the operations further comprise operating the first pump at a third pump rate to provide a third flow of the transduction media to each of the pair of the intra-capillary ports.

33. The system of claim 22, further comprising a second pump including a second pump inlet in communication with each of the cell source and the vector source, and a second pump outlet in selective communication with each of the pair of extra-capillary ports.

34. The system of claim 33, further comprising a culture media source including a culture media in communication with the first pump inlet and the second pump inlet.

35. The system of claim 34, wherein the operations further comprise:
operating the first pump at a fourth pump rate to provide a fourth flow of the culture media to one of the intra-capillary ports; and
operating the second pump at a fifth pump rate to provide a fifth flow of the culture media to each of the pair of the extra-capillary ports.

36. The system of claim 22, wherein the vector comprises at least one of a non-viral vector or a viral vector.

37. The system of claim 22, further comprising a mixer configured to receive at least one of a population of cells or a population of vector particles.

38. The system of claim 37, wherein the operations further comprise mixing, using the mixer, the population of cells with the population of vector particles to generate a mixture prior to loading the mixture into the filter module.

39. The system of claim 22, wherein the operations further comprise generating a cell therapy product comprising one or more transduced cells by:
loading the cells and the vector into the intra-capillary space, resulting in transduction of one or more of the cells in the intra-capillary space; and
harvesting a population of cells comprising the one or more transduced cells from the intra-capillary space.

40-46. (canceled)

47. A system comprising:
a filter module defining an intra-capillary space and an extra-capillary space separated from the intra-capillary space by a porous membrane, the filter module including a pair of intra-capillary ports fluidly coupled to opposite ends of the intra-capillary space and a pair of extra-capillary ports coupled to opposite ends of the extra-capillary space;
a first pump including a first pump inlet in communication with each of a cell source including cells and a vector source including a vector, and a first pump outlet in selective communication with a first intra-capillary port of the pair of intra-capillary ports;
a second pump including a second pump inlet in communication with each of the cell source and the vector source, and a second pump outlet in selective communication with a second intra-capillary port of the pair of intra-capillary ports;
a first fluid sensor disposed between the first pump outlet and the first intra-capillary port of the pair of the intra-capillary ports;
data processing hardware; and
memory hardware in communication with the data processing hardware, the memory hardware storing instructions that when executed on the data processing hardware cause the data processing hardware to perform operations according to a recipe for transducing the cells with the vector.

48. The system of claim 47, wherein the first pump is operable to provide a first flow including at least one of the cells or the vector to the first intra-capillary port and the second pump is operable to provide a second flow including at least one of the cells or the vector to the second intra-capillary port simultaneously with the first flow being provided to the first intra-capillary port.

49-51. (canceled)
